SEQUENCE LISTING (47 sequences)

SEQ ID NO: 1 - DNA, 23 bp, Artificial Sequence
  synthetic oligonucleotide
gggcccctgtggggaaacgaggg

SEQ ID NO: 2 - DNA, 23 bp, Artificial Sequence
  synthetic oligonucleotide
agggcccctgtggggaaacgagg

SEQ ID NO: 3 - DNA, 23 bp, Artificial Sequence
  synthetic oligonucleotide
ccccacaggggccctggctatgg

SEQ ID NO: 4 - DNA, 23 bp, Artificial Sequence
  synthetic oligonucleotide
agcactcacacggtgggagaagg

SEQ ID NO: 5 - protein, 118 aa, Artificial Sequence
  synthetic polypeptide
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 6 - DNA, 354 bp, Artificial Sequence
  synthetic polynucleotide
caggtgcagctgcagcagcccggcgccgaggtggtgaagcccggcgccagcgtgaagatgagctgcaaggccagcggctacaccttcaccagctactacatccactggatcaagcagacccccggccagggcctggagtgggtgggcgtgatctaccccggcaacgacgacatcagctacaaccagaagttccagggcaaggccaccctgaccgccgacaagagcagcaccaccgcctacatgcagctgagcagcctgaccagcgaggacagcgccgtgtactactgcgccagggaggtgaggctgaggtacttcgacgtgtggggccagggcaccaccgtgaccgtgagcagc

SEQ ID NO: 7 - protein, 113 aa, Artificial Sequence
  synthetic polypeptide
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKR

SEQ ID NO: 8 - DNA, 339 bp, Artificial Sequence
  synthetic polynucleotide
gagatcgtgctgacccagagccccggcagcctggccgtgagccccggcgagagggtgaccatgagctgcaagagcagccagagcgtgttcttcagcagcagccagaagaactacctggcctggtaccagcagatccccggccagagccccaggctgctgatctactgggccagcaccagggagagcggcgtgcccgacaggttcaccggcagcggcagcggcaccgacttcaccctgaccatcagcagcgtgcagcccgaggacctggccatctactactgccaccagtacctgagcagcaggaccttcggccagggcaccaagctggagatcaagagg

SEQ ID NO: 9 - protein, 19 aa, Artificial Sequence
  synthetic polypeptide
GSTSSGSGKPGSGEGSTKG

SEQ ID NO: 10 - protein, 107 aa, Artificial Sequence
  synthetic polypeptide
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 11 - protein, 89 aa, Artificial Sequence
  synthetic polypeptide
LSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 12 - protein, 38 aa, Artificial Sequence
  synthetic polypeptide
CWLTKKKYSSSVHDPNGEYMFMRAVNTAKKSRLTDVTL

SEQ ID NO: 13 - protein, 97 aa, Artificial Sequence
  synthetic polypeptide
IEVMYPPPYLDNEKSNGTIIHVKGKHLCPSPLFPGPSKPFWVLVVVGGVLACYSLLVTVAFIIFWVRSKRSRLLHSDYMFMRAVNTAKKSRLTDVTL

SEQ ID NO: 14 - protein, 113 aa, Artificial Sequence
  synthetic polypeptide
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 15 - DNA, 657 bp, Artificial Sequence
  synthetic polynucleotide
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 16 - DNA, 603 bp, Artificial Sequence
  synthetic polynucleotide
ctatcaatttttgatcctcctccttttaaagtaactcttacaggaggatatttgcatatttatgaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 17 - DNA, 627 bp, Artificial Sequence
  synthetic polynucleotide
attgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 18 - protein, 503 aa, Artificial Sequence
  synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDIYIWAPLAGTCGVLLLSLVITKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 509 aa, Artificial Sequence
  synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 552 aa, Artificial Sequence
  synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 542 aa, Artificial Sequence
  synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDFWLPIGCAAFVVVCILGCILICWLTKKKYSSSVHDPNGEYMFMRAVNTAKKSRLTDVTLTKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 459 aa, Artificial Sequence
  synthetic polypeptide
MWLQSLLLLGTVACSISIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIGSTSGSGKPGSGEGSTKGLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSALSNSIMYFSHFVPVFLPAKPTTTPAPRPPTPAPTIASQPLSLRPEASRPAAGGAVHTRGLDKPFWVLVVVGGVLACYSLLVTVAFIIFWVRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 398 aa, Artificial Sequence
  synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRS

SEQ ID NO: 24 - protein, 500 aa, Artificial Sequence
  synthetic polypeptide
MWLQSLLLLGTVACSISIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIGSTSGSGKPGSGEGSTKGLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSALSNSIMYFSHFVPVFLPAKPTTTPAPRPPTPAPTIASQPLSLRPEASRPAAGGAVHTRGLDKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 484 aa, Artificial Sequence
  synthetic polypeptide
MWLQSLLLLGTVACSISEIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIKRGSTSGSGKPGSGEGSTKGQVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMGERRRGKGHDGLYQGLSTATKDTYDALHMQALPPR

SEQ ID NO: 26 - DNA, 1568 bp, Artificial Sequence
  synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggcctggatatctacatctgggccccactggccggcacctgtggcgtgctgctgctgtctctcgtgatcaccaagagaggccggaagaagctgctgtacatcttcaagcagcccttcatgcggcccgtgcagaccacccaggaagaggacggctgtagctgccggttccccgaggaagaagaagggggctgcgagctgagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagacggaagaaccctcaggaaggcctgtataacgaactgcagaaagacaagatggccgaggcctactccgagatcggaatgaagggcgagcggagaagaggcaagggccacgatggactgtaccagggcctgagcaccgccaccaaggacacctatgacgccctgcacatgcaggccctgccccccagatgaaattcatcgacgttaactattctag

SEQ ID NO: 27 - DNA, 1586 bp, Artificial Sequence
  synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttacagcctgctcgtgacagtggccttcatcatcttttgggtgcgcagcaagcggtctagactgctgcacagcgactacatgaacatgacccccagaaggccaggccccacccggaagcactatcagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagagaagaggccgggaccctgagatgggcggcaagcccagacggaagaaccctcaggaaggcctgtataacgaactgcagaaagacaagatggccgaggcctactccgagatcggcatgaagggcgaacggcggagaggcaagggacacgatggactgtaccagggcctgagcaccgccaccaaggacacctatgacgccctgcacatgcaggccctgccccccagatgaaattcatcgacgttaactattctag

SEQ ID NO: 28 - DNA, 1715 bp, Artificial Sequence
  synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttacagcctgctcgtgacagtggccttcatcatcttttgggtgcgcagcaagcggtctagactgctgcacagcgactacatgaacatgacccccagaaggccaggccccacccggaagcactatcagccttacgcccctcccagagacttcgccgcctacagatccaagagaggccggaagaagctgctgtacatcttcaagcagcccttcatgcggcccgtgcagaccacccaggaagaggacggctgtagctgccggttccccgaggaagaagaagggggctgcgagctgagagtgaagttcagcagaagcgccgacgcccctgcctatcagcagggccagaaccagctgtacaacgagctgaacctgggcagacgggaagagtacgacgtgctggacaagagaagaggccgggaccctgagatgggcggcaagcccagacggaagaaccctcaggaaggcctgtataacgaactgcagaaagacaagatggccgaggcctactccgagatcggaatgaagggcgagcggcggagaggcaagggacacgatggactgtaccagggcctgagcaccgccaccaaggacacctatgacgccctgcacatgcaggccctgccccccagatgaaattcatcgacgttaactattctag

SEQ ID NO: 29 - DNA, 1436 bp, Artificial Sequence
  synthetic polynucleotide
ggtgtcgtgagcggccgctgaactggccaccatgtggctgcagtctctgctgctgctgggcaccgtggcctgcagcatcagcatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgccctctagattttccggcagcggctccggcaccgactacagcctgaccatctccaacctggaacaggaagatatcgctacctacttctgtcagcaaggcaacaccctgccctacaccttcggcggaggcaccaagctggaaatcggcagcacaagcggctctggcaagcctggatctggcgagggctctaccaagggcctgcaggaatctggccctggactggtggcccctagccagagcctgtctgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttatagcctgctcgtgacagtggccttcatcatcttttgggtgcgcgtgaagttcagccgcagcgccgatgcccctgcctatcagcagggacagaaccagctgtacaacgagctgaacctgggcagacgggaagagtacgacgtgctggacaagagaagaggccgggaccctgagatgggcggcaagcccagaagaaagaacccccaggaaggcctgtataacgaactgcagaaagacaagatggccgaggcctacagcgagatcggcatgaagggcgaacggcggagaggcaagggccacgatggactgtatcagggcctgagcaccgccaccaaggacacctatgacgccctgcacatgcaggctctgccccctcgctgaaattcatcgacgttaactattctag

SEQ ID NO: 30 - DNA, 1253 bp, Artificial Sequence
  synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtctagcgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttacagcctgctcgtgacagtggccttcatcatcttttgggtgcgcagcaagcggtctagactgctgcacagcgactacatgaacatgacccccagaaggccaggccccacccggaagcactatcagccttacgcccctcccagagacttcgccgcctacagaagctgaaattcatcgacgttaactattctag

SEQ ID NO: 31 - DNA, 1559 bp, Artificial Sequence
  synthetic polynucleotide
ggtgtcgtgagcggccgctgaactggccaccatgtggctgcagtctctgctgctgctgggcaccgtggcctgcagcatcagcatccagatgacccagaccaccagcagcctgagcgccagcctgggcgatagagtgaccatcagctgcagagccagccaggacatcagcaagtacctgaactggtatcagcagaaacccgacggcaccgtgaagctgctgatctaccacaccagcagactgcacagcggcgtgccctctagattttccggcagcggctccggcaccgactacagcctgaccatctccaacctggaacaggaagatatcgctacctacttctgtcagcaaggcaacaccctgccctacaccttcggcggaggcaccaagctggaaatcggcagcacaagcggctctggcaagcctggatctggcgagggctctaccaagggcctgcaggaatctggccctggactggtggcccctagccagagcctgtctgtgacctgtaccgtgtccggcgtgtccctgcctgactatggcgtgtcctggatcagacagccccccagaaagggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactactgcgccaagcactactactacggcggcagctacgccatggactactggggccagggcacaagcgtgaccgtgtctgccctgagcaacagcatcatgtacttcagccacttcgtgcccgtgtttctgcccgccaagcctaccacaacccctgcccctagacctcctaccccagcccctacaatcgccagccagcctctgtctctgaggcccgaggcttctagaccagctgctggcggagccgtgcacaccagaggactggacaagcccttctgggtgctggtggtcgtgggcggagtgctggcctgttatagcctgctcgtgacagtggccttcatcatcttttgggtgcgcagcaagcggagccggctgctgcactccgactacatgaacatgacccccagacggccaggccccacccggaaacactatcagccttacgcccctcccagagacttcgccgcctaccggtccagagtgaagttcagcagatccgccgacgcccctgcctatcagcagggacagaaccagctgtacaacgagctgaacctgggcagacgggaagagtacgacgtgctggacaagagaagaggccgggaccctgagatgggcggcaagcccagaagaaagaacccccaggaaggcctgtataacgaactgcagaaagacaagatggccgaggcctacagcgagatcggcatgaagggcgaacggcggagaggcaagggccacgatggactgtatcagggcctgagcaccgccaccaaggacacctatgacgccctgcacatgcaggctctgccccctcgctgaaattcatcgacgttaactattctag

SEQ ID NO: 32 - DNA, 1514 bp, Artificial Sequence
  synthetic polynucleotide
ggtgtcgtgagcggccgctgaactggccaccatgtggctgcagtctctgctgctgctgggcaccgtggcctgtagcatcagcgagatcgtgctgacccagagccctggctctctggctgtgtctcctggcgagcgcgtgaccatgagctgcaagagcagccagagcgtgttcttcagcagctcccagaagaactacctggcctggtatcagcagatccccggccagagccccagactgctgatctactgggccagcaccagagaaagcggcgtgcccgatagattcaccggcagcggctctggcaccgacttcaccctgacaatcagcagcgtgcagcccgaggacctggccatctactactgccaccagtacctgagcagccggacctttggccagggcaccaagctggaaatcaagcggggcagcacaagcggcagcggaaagcctggatctggcgagggctctaccaagggccaggtgcagctgcagcagcctggcgccgaagtcgtgaaacctggcgcctccgtgaagatgtcctgcaaggccagcggctacaccttcaccagctactacatccactggatcaagcagacccctggacagggcctggaatgggtgggagtgatctaccccggcaacgacgacatcagctacaaccagaagttccagggcaaggccaccctgaccgccgacaagtctagcaccaccgcctacatgcagctgtccagcctgaccagcgaggacagcgccgtgtactactgcgccagagaagtgcggctgcggtacttcgatgtgtggggccagggaaccaccgtgaccgtgtccagcatcgaagtgatgtacccccctccctacctggacaacgagaagtccaacggcaccatcatccacgtgaagggcaagcacctgtgccccagccctctgtttcctggccctagcaagcccttctgggtgctggtggtcgtgggcggagtgctggcctgttacagcctgctcgtgacagtggccttcatcatcttttgggtgcgcagcaagcggtctagactgctgcacagcgactacatgaacatgacccccagaaggccaggccccacccggaagcactatcagccttacgcccctcccagagacttcgccgcctaccggtccagagtgaagttcagcagaagcgccgacgcccctgcctatcagcagggccagaaccagctgtacaacgagctgaacctgggcagacgggaagagtacgacgtgctggacaagcggagaggcagggaccctgagatgggcggcaagcccagacggaagaaccctcaggaaggcctgtataacgaactgcagaaagacaagatggccgaggcctactccgagatcggcatgaagggcgagcggagaagaggcaagggccacgatggactgtaccagggcctgagcaccgccaccaaggacacctatgacgccctgcacatgcaggccctgccccccagatgaaattcatcgacgttaactattctag

SEQ ID NO: 33 - DNA, 850 bp, Artificial Sequence
  synthetic polynucleotide
ggtgtcgtgagcggccgctgaactggccaccatggacatgagggtccctgctcagctcctggggctcctgctgctctggctctcaggtgccagatgtgagatcgtgctgacccagagccccggcagcctggccgtgagccccggcaagagggtgaccatgagctgcaagagcagccagagcgtgttcttcagcagcagccagaagaactacctggcctggtaccagcagatccccggccagagccccaggctgctgatctactgggccagcaccagggagagcggcgtgcccgacaggttcaccggcagcggcagcggcagcggcaccgacttcaccctgaccatcagcagcgtgcagcccgaggacctggccatctactactgccaccagtacctgagcagcaggaccttcggccagggcaccaagctggagatcaagaggggcagcaccagcggcagcggcaagcccggcagcggcgagggcagcaccaagggccaggtgcagctgcagcagcccggcgccgaggtggtgaagcccggcgccagcgtgaagatgagctgcaaggccagcggctacaccttcaccagctactacatccactggatcaagcagacccccggccagggcctggagtgggtgggcgtgatctaccccggcaacgacgacatcagctacaaccagaagttccagggcaaggccaccctgaccgccgacaagagcagcaccaccgcctacatgcagctgagcagcctgaccagcgaggacagcgccgtgtactactgcgccagggaggtgaggctgaggtacttcgacgtgtggggccagggcaccaccgtgaccgtgagcagc

SEQ ID NO: 34 - DNA, 695 bp, Artificial Sequence
  synthetic polynucleotide
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacgcccctctccctcccccccccctaa

SEQ ID NO: 35 - DNA, 641 bp, Artificial Sequence
  synthetic polynucleotide
gcggccgcactatcaatttttgatcctcctccttttaaagtaactcttacaggaggatatttgcatatttatgaatcacaactttgttgccagctgaagttctggttacccataggatgtgcagcctttgttgtagtctgcattttgggatgcatacttatttgttggcttacaaaaaagaagtattcatccagtgtgcacgaccctaacggtgaatacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacgcccctctccctcccccccccctaa

SEQ ID NO: 36 - DNA, 665 bp, Artificial Sequence
  synthetic polynucleotide
gcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgttcatgagagcagtgaacacagccaaaaaatctagactcacagatgtgaccctaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacgcccctctccctcccccccccctaa

SEQ ID NO: 37 - DNA, 32 bp, Homo sapiens
ccctcgtttccccacaggggccctggctatgg

SEQ ID NO: 38 - DNA, 32 bp, Homo sapiens
gggagcaaaggggtgtccccgggaccgatacc

SEQ ID NO: 39 - DNA, 17 bp, Homo sapiens
ccccacaggggccctgg

SEQ ID NO: 40 - DNA, 32 bp, Homo sapiens
tcaccttctcccaccgtgtgagtgctggtgga

SEQ ID NO: 41 - DNA, 32 bp, Homo sapiens
agtggaagagggtggcacactcacgaccacct

SEQ ID NO: 42 - DNA, 23 bp, Artificial Sequence
  Synthetic Oligonucleotide
gggcttttctccccaaacgaggg

SEQ ID NO: 43 - DNA, 22 bp, Artificial Sequence
  Synthetic Oligonucleotide
agggctttgtggggaaacgagg

SEQ ID NO: 44 - DNA, 23 bp, Artificial Sequence
  Synthetic Oligonucleotide
ccccgcgggggccctggctatgg

SEQ ID NO: 45 - DNA, 23 bp, Artificial Sequence
  Synthetic Oligonucleotide
agcgctcgcacggtgggagaagg

SEQ ID NO: 46 - DNA, 23 bp, Artificial Sequence
  Synthetic Oligonucleotide
atcttaccttgtgttccggaggg

SEQ ID NO: 47 - DNA, 23 bp, Artificial Sequence
  Artificial Oligonucleotide
gtggtacctgctggctgaggagg

(19) United States
(12) Patent Application Publication
(10) Pub. No.: US 2024/0041923 A1
(43) Pub. Date: Feb. 8, 2024

(54) COMPOSITIONS AND METHODS FOR INHIBITION OF LINEAGE SPECIFIC ANTIGENS USING CRISPR-BASED BASE EDITOR SYSTEMS

(71) Applicant: The Trustees of Columbia University in the City of New York, New York, NY (US)

(72) Inventors: Siddhartha MUKHERJEE, New York, NY (US); Florence BOROT, New York, NY (US); Abdullah Mahmood ALI, Tenafly, NJ (US)

(21) Appl. No.: 17/928,674

(22) PCT Filed: Jun. 3, 2021
(86) PCT No.: PCT/US21/35691

§ 371 Date: Nov. 30, 2022

Related U.S. Application Data
(60) Provisional application No. 63/033,966, filed on Jun. 3, 2020.
(60) Provisional application No. 63/033,970, filed on Jun. 3, 2020.
(60) Provisional application No. 63/183,791, filed on May 4, 2021.

(51) Int. Cl. A61K 35/17; C12N 15/11; C12N 5/0775; C12N 9/22

(52) U.S. Cl. CPC A61K 35/17; C12N 15/11; C12N 5/0665; C12N 9/22; C12N 2310/20

(57) ABSTRACT

Disclosed herein are methods of administering an agent targeting a lineage-specific cell-surface antigen, e.g., CD33 or EMR2, and a population of hematopoietic cells that are altered in the expression of the lineage-specific cell-surface antigen, e.g., CD33 or EMR2, for immunotherapy of hematological malignancies. Also disclosed herein are methods of administering an agent targeting more than one lineage-specific cell-surface antigen, and a population of hematopoietic cells that are altered in the expression of more than one lineage-specific cell-surface antigen, for immunotherapy of hematological malignancies. Cells comprising mutations in CD33, or EMR2, or more than one lineage-specific cell-surface antigen are also provided, as are methods of producing such cells using CRISPR-based base editor systems.

This application claims priority to: U.S. Ser. No. 63/033,966 filed Jun. 3, 2020; U.S. Ser. No. 63/033,970 filed Jun. 3, 2020; and U.S. Ser. No. 63/183,791 filed May 4, 2021, the entire contents of each of which is incorporated herein by reference.

### SEQUENCE LISTING

The instant application contains a Sequence Listing which has been submitted electronically in ASCII format and is hereby incorporated by reference in its entirety.

### BACKGROUND

The emergence of targeted immunotherapy has opened new possibilities, demonstrating tremendous success for patients with lymphoblastic leukemia. However, clinical studies with chimeric antigen T-cell therapy (CAR-T) have shown limited success beyond CD19-targeted immunotherapy, due to lack of unique targetable cell surface antigens. In fact, the success of CD19 immunotherapy lies on B cell aplasia which can be managed with immunoglobulin supplements.

However, any targeted immunotherapy approach requires antigens that are uniquely or preferentially expressed on cancerous cells. Indeed, in order for an antigen to be an ideal candidate for immunotherapy, it should be unique to cancer cells, indispensable for their survival and not expressed on normal cells. To date, no truly tumor specific antigen to cancerous cells has been found. For cancers, where such an ideal antigen is absent, a novel approach combining the targeting of an antigen that is lineage specific and overexpressed by malignant cells with the transplantation of genetically engineered stem cells lacking that lineage specific antigen (LSA) was considered.

In previous published work and patent (U.S. Pat. No. 10,137,155), it was demonstrated that the approach of using CD33-targeted CAR-T cells and/or the AD gemtuzumab ozogamicin (GO) was able to rescue CD33+ Acute Myeloid Leukemia-bearing mice without affecting the engraftment and repopulation of CD33 knockout (KO) hematopoietic stem cells that had been engineered using CRISPR/Cas9 technology to delete the lineage specific antigen.

However, genome editing with CRISPR/WT Cas9 induces DNA double strands breaks (DSBs) that have been associated with p53-mediated DNA damage response and chromosomal translocation.

Thus, a different approach is necessary to generate targeting-resistant hematopoietic stem cells that does not cause off target effects and debilitating immunosuppressive side effects.

### SUMMARY

The present disclosure is based, at least in part, on the discovery that agents comprising an antigen-binding fragment that binds a lineage-specific cell-surface antigen (e.g., immune cells expressing a chimeric receptor that targets a lineage-specific cell-surface antigen) selectively cause cell death of cells expressing the lineage-specific cell-surface antigen, whereas cells that are deficient for the antigen (e.g., genetically engineered hematopoietic cells) evade cell death caused thereby. Based on such findings, it would have been expected that immunotherapies involving the combination of an agent targeting a lineage-specific cell-surface antigen, for example, CAR-T cells targeting CD33, and hematopoietic cells that are deficient or altered in the lineage-specific cell-surface antigens (e.g., CD33) would provide an efficacious method of treatment for hematopoietic malignancies.

Described herein is the use of CRISPR-based base editors to generate targeting-resistant hematopoietic stem cells that are HSCs lacking the epitope or containing a modified epitope targeted by therapeutic agents including antibodies and chimeric antigen receptor T-cells (CAR-Ts). This approach will further allow treatment of patients with hematologic malignancies with a combination of targeted immunotherapy, and transplantation of targeting-resistant hematopoietic stem cells generated with base editors and circumvent the potential genotoxicity of CRISPR/WT Cas9 genome editing. This novel approach allows high editing efficiency of HSCs/HSPCs using CRISPR-based cytosine and adenine base editors (CBEs and ABEs). The CBEs and ABEs are Cas9 nickase fused to a cytidine or adenosine deaminase, respectively, enabling precise base substitutions at targeted regions without generating DSBs. Because they elude DSBs, base editors are considered to be safer editing tools that eliminate undesired indels, translocations or rearrangements resulting from DSBs.

In one aspect the present disclosure provides genetically engineered hematopoietic cells (e.g., HSCs) that are deficient in a lineage-specific cell-surface antigen, which presents on the hematopoietic cell before genetic engineering. In some embodiments, the whole or a portion of an endogenous gene encoding the lineage-specific cell-surface antigen is deleted, for example by genome editing using a base editor (e.g., involving a CRISPR-based base editor system). In some embodiments, the CRISPR-based base editor system deletes an exon from the endogenous gene encoding the lineage-specific cell-surface antigen. In some embodiments, the CRISPR-based base editor system results in a nucleotide substitution in an endogenous gene encoding the lineage-specific cell-surface antigen. In some embodiments, the nucleotide substitution is within a sequence encoding a splice element, wherein the nucleotide substitution results in an alternative splicing of a transcript encoded by the gene. In some embodiments, the CRISPR-based base editor system targets a splice element in an endogenous gene, wherein the CRISPR-based base editor system results in an alternative splicing of a transcript encoded by the gene. In some embodiments, the alternative splicing causes an exon encoding an epitope to be skipped. In some embodiments, the alternative splicing causes an exon encoding an epitope to be extended. In some embodiments, the alternative splicing induces an early codon termination. In some embodiments, the splice element is a splice donor, a splice acceptor, a splice enhancer or a splice silencer. In some embodiments, the base editor is a cytosine base editor. In some embodiments, the base editor is an adenine base editor. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”.

In some embodiments, the lineage-specific cell-surface antigen is CD33. In some embodiments, a splice acceptor or exonic splicing enhancer site in exon 2 of CD33 is altered. In some embodiments, the nucleotide sequence of the intron 1/exon 2 junction of CD33 is altered. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”. In some embodiments, the gRNA sequence hybridizes to a portion of the nucleotide sequence encoding CD33. In some embodiments, the gRNA sequence targets a splice acceptor or exonic splicing enhancer site in exon 2 of the nucleotide sequence encoding CD33. In some embodiments, the gRNA sequence targets a SNP of CD33, rs12459419. In some embodiments, the gRNA sequence targets the intron 1/exon 2 junction of CD33. In some embodiments, the gRNA sequence targets a nucleotide sequence comprising SEQ ID NO: 37. In some embodiments, the gRNA has a sequence comprising any one of SEQ ID NOs: 1-3.

In some embodiments, the lineage-specific cell-surface antigen is EMR2. In some embodiments, a splice donor site in exon 13 of EMR2 is altered. In some embodiments, the nucleotide sequence of the intron 12/exon 13 junction of EMR2 is altered. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”. In some embodiments, the gRNA sequence hybridizes to a portion of the nucleotide sequence encoding EMR2. In some embodiments, the gRNA sequence targets a splice donor site in exon 13 of the nucleotide sequence encoding EMR2. In some embodiments, the gRNA sequence targets the intron 12/exon 13 junction of EMR2. In some embodiments, the gRNA sequence targets a nucleotide sequence comprising SEQ ID NO: 40. In some embodiments, the gRNA has a sequence comprising any one of SEQ ID NOs: 4 or 46-47.

In some embodiments, the hematopoietic cell is a hematopoietic stem cell (e.g., CD34⁺/CD33^{Δ2} cell or CD34⁺/EMRA^{Δ13}).

In some embodiments, the hematopoietic stem cells can be obtained from bone marrow cells or peripheral blood mononuclear cells (PBMCs).

In some aspects, the disclosure provides compositions and methods for the combined inhibition of a first lineage-specific cell surface antigen and at least one additional lineage-specific cell-surface antigen, i.e., a second lineage-specific cell-surface antigen, a third lineage-specific cell-surface antigen, a fourth lineage-specific cell surface antigen, etc.

In some embodiments, the additional lineage-specific cell-surface antigen is also deleted or inhibited in the hematopoietic cells using a CRISPR-based base editor system. In some embodiments, the whole or a portion of an endogenous gene encoding the lineage-specific cell-surface antigen(s) is deleted, for example by genome editing using a base editor (e.g., involving a CRISPR-based base editor system). In some embodiments, the CRISPR-based base editor system deletes an exon from the endogenous gene encoding the lineage-specific cell-surface antigen(s). In some embodiments, the CRISPR-based base editor system results in a nucleotide substitution in an endogenous gene encoding the lineage-specific cell-surface antigen(s). In some embodiments, the nucleotide substitution is within a sequence encoding a splice element, wherein the nucleotide substitution results in an alternative splicing of a transcript encoded by the gene(s). In some embodiments, the CRISPR-based base editor system targets a splice element in an endogenous gene(s), wherein the CRISPR-based base editor system results in an alternative splicing of a transcript encoded by the gene(s). In some embodiments, the alternative splicing causes an exon encoding an epitope to be skipped. In some embodiments, the alternative splicing causes an exon encoding an epitope to be extended. In some embodiments, the alternative splicing induces an early codon termination. In some embodiments, the splice element is a splice donor, a splice acceptor, a splice enhancer or a splice silencer. In some embodiments, the base editor is a cytosine base editor. In some embodiments, the base editor is an adenine base editor. In some embodiments, the base editor is an adenine base editor. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”.

In some embodiments, the first lineage-specific cell-surface antigen is CD33. In some embodiments, the at least one additional lineage-specific cell-surface antigen or second lineage-specific cell-surface antigen is EMR2.

In some embodiments, more than one additional lineage-specific cell-surface antigen is deleted or inhibited using a CRISPR-based base editor system. In some embodiments, the more than one additional lineage-specific cell-surface antigen is different than CD33 and EMR2.

In some embodiments, the hematopoietic cell is a hematopoietic stem cell (e.g., CD34⁺/CD33^{Δ2}/EMR2^{Δ13} cell).

In some embodiments, the hematopoietic stem cells can be obtained from bone marrow cells or peripheral blood mononuclear cells (PBMCs).

In some aspects, the disclosure provides a genetically engineered hematopoietic stem or progenitor cell, which comprises an altered splice acceptor or exonic splicing enhancer site in the exon 2 of an endogenous CD33 gene. One aspect of the present disclosure provides a genetically engineered hematopoietic stem and/or progenitor cell, wherein the genetically engineered hematopoietic stem and/or progenitor cell has a reduced expression level of an epitope encoded by exon 2 of CD33 as compared with a wildtype counterpart. In some embodiments, the genetically engineered hematopoietic stem and/or progenitor cell expresses less than 10% of the epitope of the CD33 expressed by the wild-type counterpart. In some embodiments, the genetically engineered hematopoietic stem and/or progenitor cell does not express the epitope of CD33. In some embodiments, a splice acceptor or exonic splicing enhancer site in exon 2 of CD33 is altered. In some embodiments, the nucleotide sequence of the intron 1/exon 2 junction of CD33 is altered. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”. In some embodiments, the genetically engineered hematopoietic stem and/or progenitor cell is CD34⁺. In some embodiments, the genetically engineered hematopoietic stem and/or progenitor cell is from bone marrow cells or peripheral blood mononuclear cells of a subject (e.g., a human patient having a hematopoietic malignancy, or a healthy donor).

In some aspects, the disclosure provides a genetically engineered hematopoietic stem or progenitor cell, which comprises an altered splice donor site in the exon 13 of an endogenous EMR2 gene. One aspect of the present disclosure provides a genetically engineered hematopoietic stem and/or progenitor cell, wherein the genetically engineered hematopoietic stem and/or progenitor cell has a reduced expression level of an epitope encoded by exon 13 of EMR2 as compared with a wildtype counterpart. One aspect of the present disclosure provides a genetically engineered hematopoietic stem and/or progenitor cell, wherein the altered splice donor site induces an early codon termination and production of a mutated or truncated EMR2 as compared with a wildtype counterpart. In some embodiments, the genetically engineered hematopoietic stem and/or progenitor cell does not express the epitope of EMR2. In some embodiments, a splice donor site in exon 13 of EMR2 is altered. In some embodiments, the nucleotide sequence of the intron 12/exon 13 junction of EMR2 is altered. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”. In some embodiments, the genetically engineered hematopoietic stem and/or progenitor cell is CD34⁺. In some embodiments, the genetically engineered hematopoietic stem and/or progenitor cell is from bone marrow cells or peripheral blood mononuclear cells of a subject (e.g., a human patient having a hematopoietic malignancy, or a healthy donor).

In some aspects, the disclosure provides a genetically engineered hematopoietic stem or progenitor cell, which comprises an altered splice acceptor or exonic splicing enhancer site in the exon 2 of an endogenous CD33 gene and an altered splice element in an exon in at least one additional lineage-specific cell-surface antigen. One aspect of the present disclosure provides a genetically engineered hematopoietic stem and/or progenitor cell, wherein the genetically engineered hematopoietic stem and/or progenitor cell has a reduced expression level of an epitope encoded by an exon of CD33 and/or the at least one additional lineage-specific cell-surface antigen as compared with a wildtype counterpart. In some embodiments, the genetically engineered hematopoietic stem and/or progenitor cell expresses less than 10% of the epitope encoded by the exon of CD33 and/or the at least one additional lineage-specific cell-surface antigen expressed by the wild-type counterpart. In some embodiments, the genetically engineered hematopoietic stem and/or progenitor cell is CD34⁺. In some embodiments, the genetically engineered hematopoietic stem and/or progenitor cell is from bone marrow cells or peripheral blood mononuclear cells of a subject (e.g., a human patient having a hematopoietic malignancy, or a healthy donor).

In some aspects, the disclosure provides a genetically engineered hematopoietic stem or progenitor cell, which comprises an altered splice acceptor or exonic splicing enhancer site in the exon 2 of an endogenous CD33 gene and an altered splice donor site in the exon 13 of an endogenous EMR2. One aspect of the present disclosure provides a genetically engineered hematopoietic stem and/or progenitor cell, wherein the genetically engineered hematopoietic stem and/or progenitor cell has a reduced expression level of an epitope encoded by exon 2 of CD33 and/or an epitope encoded by exon 13 of EMR2 as compared with a wildtype counterpart. In some embodiments, the genetically engineered hematopoietic stem and/or progenitor cell expresses less than 10% of the epitope encoded by exon 2 of CD33 and/or the epitope encoded by exon 13 of EMR2 expressed by the wild-type counterpart. In some embodiments, the alternative splicing induces an early codon termination in EMR2 production of a mutated or truncated EMR2 as compared with a wildtype counterpart. In some embodiments, the genetically engineered hematopoietic stem and/or progenitor cell is CD34⁺. In some embodiments, the genetically engineered hematopoietic stem and/or progenitor cell is from bone marrow cells or peripheral blood mononuclear cells of a subject (e.g., a human patient having a hematopoietic malignancy, or a healthy donor).

The disclosure, in some embodiments, also provides a cell population comprising a plurality of the genetically engineered hematopoietic stem and/or progenitor cells described herein.

In another aspect, the present disclosure provides a method of producing a genetically engineered hematopoietic stem and/or progenitor cell, comprising (i) providing a hematopoietic stem and/or progenitor cell, and (ii) introducing into the cell (a) a guide RNA (gRNA) that that targets the nucleotide sequence encoding a lineage-specific cell-surface antigen, and (b) a catalytically impaired Cas protein fused to a DNA-modifying enzyme, i.e., Cas9 nickase, fused to a cytosine or adenosine deaminase (base editor), thereby producing a genetically engineered hematopoietic stem and/or progenitor cell.

In another aspect, the present disclosure provides a method of producing a genetically engineered hematopoietic stem and/or progenitor cell, comprising (i) providing a hematopoietic stem and/or progenitor cell, and (ii) introducing into the cell (a) a guide RNA (gRNA) that comprises a targeting domain targeting a nucleotide sequence within the genome of the hematopoietic stem or progenitor cell that comprises a splice element, and (b) a catalytically impaired Cas protein fused to a DNA-modifying enzyme, i.e., Cas9 nickase, fused to a cytosine or adenosine deaminase (base editor), thereby producing a genetically engineered hematopoietic stem and/or progenitor cell.

In some embodiments, the method deletes an exon from the endogenous gene encoding the lineage-specific cell-surface antigen. In some embodiments, the method results in a nucleotide substitution in an endogenous gene encoding the lineage-specific cell-surface antigen. In some embodiments, the nucleotide substitution is within a sequence encoding a splice element, wherein the nucleotide substitution results in an alternative splicing of a transcript encoded by the gene. In some embodiments, the method targets a splice element in an endogenous gene, wherein the method results in an alternative splicing of a transcript encoded by the gene. In some embodiments, the alternative splicing causes an exon encoding an epitope to be skipped. In some embodiments, the alternative splicing causes an exon encoding an epitope to be extended. In some embodiments, the splice element is a splice donor, a splice acceptor, a splice enhancer or a splice silencer. In some embodiments, the base editor is a cytosine base editor. In some embodiments, the base editor is an adenine base editor. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”.

In some embodiments, the lineage-specific cell-surface antigen is CD33. In some embodiments, the gRNA targets a nucleotide sequence flanking exon 2 of CD33. In some embodiments, the gRNA comprises a nucleotide sequence that is at least 90% identical to SEQ ID NO: 1, SEQ ID NO: 2, and/or SEQ ID NO: 3. In some embodiments, the gRNA sequence targets the intron 1/exon 2 junction of CD33. In some embodiments, the gRNA sequence targets a nucleotide sequence comprising SEQ ID NO: 37. In some embodiments, the gRNA and a catalytically impaired Cas protein fused to a DNA-modifying enzyme are encoded on one vector, which is introduced into the cell. In some embodiments, the vector is a viral vector. In some embodiments, the gRNA and a catalytically impaired Cas protein fused to a DNA-modifying enzyme are introduced into the cell as a pre-formed ribonucleoprotein complex. In some embodiments, the ribonucleoprotein complex is introduced into the cell via electroporation.

In some embodiments, the lineage-specific cell-surface antigen is EMR2. In some embodiments, the gRNA targets a nucleotide sequence flanking exon 13 of EMR2. In some embodiments, the gRNA comprises a nucleotide sequence that is at least 90% identical to SEQ ID NO: 4, SEQ ID NO: 46 and/or SEQ ID NO: 47. In some embodiments, the gRNA sequence targets the intron 12/exon 13 junction of EMR2. In some embodiments, the gRNA sequence targets a nucleotide sequence comprising SEQ ID NO: 40. In some embodiments, the gRNA and a catalytically impaired Cas protein fused to a DNA-modifying enzyme are encoded on one vector, which is introduced into the cell. In some embodiments, the vector is a viral vector. In some embodiments, the gRNA and a catalytically impaired Cas protein fused to a DNA-modifying enzyme are introduced into the cell as a pre-formed ribonucleoprotein complex. In some embodiments, the ribonucleoprotein complex is introduced into the cell via electroporation.

In another aspect, the present disclosure provides a method of producing a genetically engineered hematopoietic stem and/or progenitor cell, comprising (i) providing a hematopoietic stem and/or progenitor cell, and (ii) introducing into the cell (a) a guide RNA (gRNA) that targets the nucleotide sequence encoding a first lineage-specific cell-surface antigen and (b) a catalytically impaired Cas protein fused to a DNA-modifying enzyme, i.e., Cas9 nickase fused to a cytosine or adenosine deaminase (base editor), and further comprising introducing into the cell a second a guide RNA (gRNA) that targets an at least one additional lineage-specific cell-surface antigen, and (b) a catalytically impaired Cas protein fused to a DNA-modifying enzyme, i.e., Cas9 nickase fused to a cytosine or adenosine deaminase (base editor), thereby producing a genetically engineered hematopoietic stem and/or progenitor cell.

In another aspect, the present disclosure provides a method of producing a genetically engineered hematopoietic stem and/or progenitor cell, comprising (i) providing a hematopoietic stem and/or progenitor cell, and (ii) introducing into the cell (a) a guide RNA (gRNA) that comprises a targeting domain targeting a nucleotide sequence within the genome of the hematopoietic stem or progenitor cell that comprises a splice element of a first lineage-specific cell-surface antigen, and (b) a catalytically impaired Cas protein fused to a DNA-modifying enzyme, i.e., Cas9 nickase, fused to a cytosine or adenosine deaminase (base editor), and further comprising introducing into the cell a second a guide RNA (gRNA) that comprises a targeting domain targeting a nucleotide sequence within the genome of the hematopoietic stem or progenitor cell of an at least one additional lineage-specific cell-surface antigen, and (b) a catalytically impaired Cas protein fused to a DNA-modifying enzyme, i.e., Cas9 nickase fused to a cytosine or adenosine deaminase (base editor), thereby producing a genetically engineered hematopoietic stem and/or progenitor cell.

In some embodiments, the lineage-specific cell-surface antigen is CD33. In some embodiments, the gRNA targets a nucleotide sequence flanking exon 2 of CD33. In some embodiments, the gRNA comprises a nucleotide sequence that is at least 90% identical to SEQ ID NO: 1, SEQ ID NO: 2, and/or SEQ ID NO: 3. In some embodiments, the gRNA sequence targets the intron 1/exon 2 junction of CD33. In some embodiments, the gRNA sequence targets a nucleotide sequence comprising SEQ ID NO: 37.

In some embodiments, the gRNA and a catalytically impaired Cas protein fused to a DNA-modifying enzyme are encoded on one vector, which is introduced into the cell. In some embodiments, the vector is a viral vector. In some embodiments, the gRNA and a catalytically impaired Cas protein fused to a DNA-modifying enzyme are introduced into the cell as a pre-formed ribonucleoprotein complex. In some embodiments, the ribonucleoprotein complex is introduced into the cell via electroporation.

In some embodiments, the at least one additional lineage-specific cell-surface antigen is EMR2. In some embodiments, the gRNA targets a nucleotide sequence flanking exon 13 of EMR2. In some embodiments, the gRNA comprises a nucleotide sequence that is at least 90% identical to SEQ ID NO: 4, SEQ ID NO: 46 and/or SEQ ID NO: 47. In some embodiments, the gRNA sequence targets the intron 12/exon 13 junction of EMR2. In some embodiments, the gRNA sequence targets a nucleotide sequence comprising SEQ ID NO: 40. In some embodiments, the gRNA and a catalytically impaired Cas protein fused to a DNA-modifying enzyme are encoded on one vector, which is introduced into the cell. In some embodiments, the vector is a viral vector. In some embodiments, the gRNA and a catalytically impaired Cas protein fused to a DNA-modifying enzyme are introduced into the cell as a pre-formed ribonucleoprotein complex. In some embodiments, the ribonucleoprotein complex is introduced into the cell via electroporation.

The present disclosure also provides, in some aspects, use of a gRNA described herein for reducing expression of an epitope of a lineage-specific cell-surface antigen in a sample of hematopoietic cells stem or progenitor cells using a CRISPR-based base editor system.

The present disclosure also provides, in some aspects, use of a CRISPR-based base editor system for reducing expression of an epitope of a lineage-specific cell-surface antigen in a sample of hematopoietic cells stem or progenitor cells.

The present disclosure also provides, in some aspects, use of a gRNA described herein for reducing expression of an epitope of CD33 in a sample of hematopoietic cells stem or progenitor cells using a CRISPR-based base editor system.

The present disclosure also provides, in some aspects, use of a CRISPR-based base editor system for reducing expression of an epitope of CD33 in a sample of hematopoietic cells stem or progenitor cells.

The present disclosure also provides, in some aspects, use of a gRNA described herein for reducing expression of an epitope of EMR2 in a sample of hematopoietic cells stem or progenitor cells using a CRISPR-based base editor system.

The present disclosure also provides, in some aspects, use of a CRISPR-based base editor system for reducing expression of an epitope of EMR2 in a sample of hematopoietic cells stem or progenitor cells.

The present disclosure also provides, in some aspects, use of a gRNA described herein for reducing expression of CD33 and at least one additional lineage-specific cell-surface antigen in a sample of hematopoietic cells stem or progenitor cells using a CRISPR-based base editor system.

The present disclosure also provides, in some aspects, use of a CRISPR-based base editor system for reducing expression of CD33 and at least one additional lineage-specific cell-surface antigen in a sample of hematopoietic cells stem or progenitor cells.

In some embodiments, the at least one additional lineage specific antigen is EMR2.

In some embodiments the gRNA is a single-molecule guide RNA (sgRNA).

In some embodiments, the hematopoietic stem and/or progenitor cell is CD34⁺. In some embodiments, the hematopoietic stem and/or progenitor cell is from bone marrow cells or peripheral blood mononuclear cells (PBMCs) of a subject. In some embodiments, the subject has a hematopoietic disorder. In some embodiments, the subject is a healthy HLA-matched donor.

The disclosure, in some embodiments, provides a genetically engineered hematopoietic stem and/or progenitor cell, which is produced by a method described herein.

In another aspect, the present disclosure provides a method of treating a hematopoietic disorder, comprising administering to a subject in need thereof an effective amount of the genetically engineered hematopoietic stem and/or progenitor cell or the cell population described herein. In some embodiments, the hematopoietic disorder is a hematopoietic malignancy.

In some embodiments, the method further comprises administering to the subject an effective amount of an agent that targets CD33, wherein the agent comprises an antigen-binding fragment that binds CD33. In some embodiments, the agent that targets CD33 is an immune cell expressing a chimeric antigen receptor (CAR), which comprises the antigen-binding fragment that binds CD33.

In some aspects, the present disclosure provides a genetically engineered hematopoietic stem or progenitor cell described herein or a cell population described herein for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets CD33, wherein the agent comprises an antigen-binding fragment that binds CD33.

In some aspects, the present disclosure provides an agent that targets CD33, wherein the agent comprises an antigen-binding fragment that binds CD33, for use in treating a hematopoietic disorder, wherein the treating comprises administering to a subject in need thereof an effective amount of the agent that targets CD33, and further comprises administering to the subject an effective amount of a genetically engineered hematopoietic stem or progenitor cell described herein or a cell population described herein.

A combination of a genetically engineered hematopoietic stem or progenitor cell described herein or a cell population described herein, and an agent that targets CD33, wherein the agent comprises an antigen-binding fragment that binds CD33, for use in treating a hematopoietic disorder, wherein the treating comprises administering to a patient in need thereof an effective amount of the genetically engineered hematopoietic stem or progenitor cell or the cell population, and the agent that binds CD33.

In some embodiments, the genetically engineered hematopoietic stem or progenitor cell or the cell population is administered concomitantly with the agent that targets CD33. In some embodiments, the genetically engineered hematopoietic stem or progenitor cell or the cell population is administered prior to the agent that targets CD33. In some embodiments, the agent that targets CD33 is administered prior to the genetically engineered hematopoietic stem or progenitor cell or the cell population.

In some embodiments, the immune cell is a T cell. In some embodiments, the immune cells, the genetically engineered hematopoietic stem and/or progenitor cell, or both, are allogeneic. In some embodiments, the immune cells, the genetically engineered hematopoietic stem and/or progenitor cell, or both, are autologous. In some embodiments, the antigen-binding fragment in the chimeric receptor is a single-chain antibody fragment (scFv) that specifically binds human CD33.

In some embodiments, the method further comprises administering to the subject an effective amount of an agent that targets EMR2, wherein the agent comprises an antigen-binding fragment that binds EMR2. In some embodiments, the agent that targets EMR2 is an immune cell expressing a chimeric antigen receptor (CAR), which comprises the antigen-binding fragment that binds EMR2.

In some aspects, the present disclosure provides a genetically engineered hematopoietic stem or progenitor cell described herein or a cell population described herein for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets EMR2, wherein the agent comprises an antigen-binding fragment that binds EMR2.

In some aspects, the present disclosure provides an agent that targets EMR2, wherein the agent comprises an antigen-binding fragment that binds EMR2, for use in treating a hematopoietic disorder, wherein the treating comprises administering to a subject in need thereof an effective amount of the agent that targets EMR2, and further comprises administering to the subject an effective amount of a genetically engineered hematopoietic stem or progenitor cell described herein or a cell population described herein.

A combination of a genetically engineered hematopoietic stem or progenitor cell described herein or a cell population described herein, and an agent that targets EMR2, wherein the agent comprises an antigen-binding fragment that binds EMR2, for use in treating a hematopoietic disorder, wherein the treating comprises administering to a patient in need thereof an effective amount of the genetically engineered hematopoietic stem or progenitor cell or the cell population, and the agent that binds EMR2.

In some embodiments, the genetically engineered hematopoietic stem or progenitor cell or the cell population is administered concomitantly with the agent that targets EMR2. In some embodiments, the genetically engineered hematopoietic stem or progenitor cell or the cell population is administered prior to the agent that targets EMR2. In some embodiments, the agent that targets EMR2 is administered prior to the genetically engineered hematopoietic stem or progenitor cell or the cell population.

In some embodiments, the immune cell is a T cell. In some embodiments, the immune cells, the genetically engineered hematopoietic stem and/or progenitor cell, or both, are allogeneic. In some embodiments, the immune cells, the genetically engineered hematopoietic stem and/or progenitor cell, or both, are autologous. In some embodiments, the antigen-binding fragment in the chimeric receptor is a single-chain antibody fragment (scFv) that specifically binds human EMR2.

In some embodiments, the method further comprises administering to the subject an effective amount of an agent that targets CD33, wherein the agent comprises an antigen-binding fragment that binds CD33, and an effective amount of an agent that targets at least one additional lineage-specific cell-surface antigen, wherein the agent comprises an antigen-binding fragment that binds the at least one additional lineage-specific cell-surface antigen. In some embodiments, the agent that targets CD33 is an immune cell expressing a chimeric antigen receptor (CAR), which comprises the antigen-binding fragment that binds CD33. In some embodiments, the agent that targets least one additional lineage-specific cell-surface antigen is an immune cell expressing a chimeric antigen receptor (CAR), which comprises the antigen-binding fragment that binds the at least one additional lineage-specific cell-surface antigen. In some embodiments, the agent that targets the CD33 and the agent that targets the at least one additional lineage-specific cell-surface antigen is an immune cell. In some embodiments, the agent that targets CD33 and the at least one additional lineage-specific cell-surface antigen is an immune cell expressing a chimeric antigen receptor (CAR), which comprises the antigen-binding fragment that binds CD33 and the at least one additional lineage-specific cell-surface antigen.

In some aspects, the present disclosure provides a genetically engineered hematopoietic stem or progenitor cell described herein or a cell population described herein for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets CD33, wherein the agent comprises an antigen-binding fragment that binds CD33, and an effective amount of an agent that targets at least one additional lineage-specific cell-surface antigen, wherein the agent comprises an antigen-binding fragment that binds the at least one additional lineage-specific cell-surface antigen.

In some aspects, the present disclosure provides an agent that targets CD33, wherein the agent comprises an antigen-binding fragment that binds CD33, and an agent that targets at least one additional lineage-specific cell-surface antigen, wherein the agent comprises an antigen-binding fragment that binds the at least one additional lineage-specific cell-surface antigen for use in treating a hematopoietic disorder, wherein the treating comprises administering to a subject in need thereof an effective amount of the agent that targets CD33, and an effective amount of an agent that targets at least one additional lineage-specific cell-surface antigen, wherein the agent comprises an antigen-binding fragment that binds the at least one additional lineage-specific cell-surface antigen and further comprises administering to the subject an effective amount of a genetically engineered hematopoietic stem or progenitor cell described herein or a cell population described herein.

A combination of a genetically engineered hematopoietic stem or progenitor cell described herein or a cell population described herein, and an agent that targets CD33, wherein the agent comprises an antigen-binding fragment that binds CD33, and an effective amount of an agent that targets at least one additional lineage-specific cell-surface antigen, wherein the agent comprises an antigen-binding fragment that binds the at least one additional lineage-specific cell-surface antigen for use in treating a hematopoietic disorder, wherein the treating comprises administering to a patient in need thereof an effective amount of the genetically engineered hematopoietic stem or progenitor cell or the cell population, and the agent that binds CD33 and the agent that binds the at least one additional lineage-specific cell-surface antigen.

In some embodiments, the agent that target CD33 and comprises an antigen-binding fragment that binds CD33, and the agent that targets at least one additional lineage-specific cell-surface antigen and comprises an antigen-binding fragment that binds the at least one additional lineage-specific cell-surface antigen are the same agent. In some embodiments, the agent is an immune cell that targets CD33 and the at least one additional lineage-specific cell-surface antigen.

In some embodiments, the genetically engineered hematopoietic stem or progenitor cell or the cell population is administered concomitantly with the agent that targets CD33 and the agent that targets at least one additional lineage-specific cell-surface antigen. In some embodiments, the genetically engineered hematopoietic stem or progenitor cell or the cell population is administered prior to the agent that targets CD33 and the agent that targets the at least one additional lineage-specific cell-surface antigen. In some embodiments, the agent that targets CD33 and the agent that targets at least one additional lineage-specific cell-surface antigen is administered prior to the genetically engineered hematopoietic stem or progenitor cell or the cell population. In some embodiments, the agent that targets CD33 is administered prior to the genetically engineered hematopoietic stem or progenitor cell or the cell population and the agent that targets the at least one additional lineage-specific cell-surface antigen is administered after the genetically engineered hematopoietic stem or progenitor cell or the cell population. In some embodiments, the agent that targets the at least one additional lineage-specific cell-surface antigen is administered prior to the genetically engineered hematopoietic stem or progenitor cell or the cell population and the agent that targets CD33 is administered after the genetically engineered hematopoietic stem or progenitor cell or the cell population.

In some embodiments, the immune cell is a T cell. In some embodiments, the immune cells, the genetically engineered hematopoietic stem and/or progenitor cell, or both, are allogeneic. In some embodiments, the immune cells, the genetically engineered hematopoietic stem and/or progenitor cell, or both, are autologous. In some embodiments, the antigen-binding fragment in the chimeric receptor is a single-chain antibody fragment (scFv) that specifically binds human CD33 and/or the at least one additional lineage-specific cell-surface antigen. In some embodiments, the antigen-binding fragment in the chimeric receptor is a single-chain antibody fragment (scFv) that specifically binds a human at least one additional lineage-specific cell-surface antigen.

In some embodiments, the at least one additional lineage-specific cell-surface antigen is EMR2.

In some embodiments, the agent that targets CD33, and/or EMR2 and/or an additional lineage-specific cell-surface antigen targets an epitope that is altered, reduced in expression or deleted in the genetically engineered hematopoietic stem or progenitor cell or the cell population described herein and administered in conjunction with the agent.

In some embodiments, the subject is a human patient having Hodgkin's lymphoma, non-Hodgkin's lymphoma, leukemia, or multiple myeloma. In some embodiments, the subject is a human patient having leukemia, which is acute myeloid leukemia, chronic myelogenous leukemia, acute lymphoblastic leukemia, or chronic lymphoblastic leukemia.

Features of the compositions and methods herein are also described in the following enumerated embodiments.

Features of the compositions and methods herein are also described in the following enumerated embodiments.
1. A genetically engineered hematopoietic stem or progenitor cell, which comprises at least one nucleotide substitution in a gene encoding a lineage-specific antigen, wherein the nucleotide substitution is comprised within a sequence encoding a splice element, and results in an alternative splicing of a transcript encoded by the gene, wherein the alternative splicing causes a reduced expression level of an epitope encoded by the gene as compared with a wild-type counterpart cell, and wherein the epitope is targeted by an immunotherapeutic agent.
2. The genetically engineered hematopoietic stem or progenitor cell of any of the preceding embodiments, wherein the splice element is chosen from the group consisting of a splice acceptor, splice donor, splice enhancer and splice silencer.
3. The genetically engineered hematopoietic stem or progenitor cell of embodiments 1 or 2, wherein alternative splicing causes an exon encoding an epitope to be skipped.
4. The genetically engineered hematopoietic stem or progenitor cell of any of the preceding embodiments, wherein alternative splicing causes an exon encoding an epitope to be extended.
5. The genetically engineered hematopoietic stem or progenitor cell of any of the preceding embodiments, wherein the nucleotide substitution is a “C” to an “T” or a “G” to an “A”.
6. The genetically engineered hematopoietic stem or progenitor cell of any of the preceding embodiments, wherein the nucleotide substitution is a “A” to an “G” or a “T” to a “C”.
7. The genetically engineered hematopoietic stem or progenitor cell of any of the preceding embodiments, wherein the nucleotide substitution is made using a CRISPR-based base editor system.
8. The genetically engineered hematopoietic stem or progenitor cell of any of the preceding embodiments, wherein the reduced expression level of the epitope is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
9. The genetically engineered hematopoietic stem or progenitor cell of embodiment 8, wherein the cell differentiated from the hematopoietic stem or progenitor cell is a myeloblast, monoblast, monocyte, macrophage, or natural killer cell.
10. A genetically engineered hematopoietic stem or progenitor cell, which comprises an altered splice acceptor or exonic splicing enhancer site in the exon 2 of an endogenous CD33 gene, wherein the alteration results in a reduced expression level of an epitope encoded by exon 2 of CD33 as compared with a wild-type counterpart cell.
11. A genetically engineered hematopoietic stem or progenitor cell, which comprises an altered splice acceptor or exonic splicing enhancer site in the exon 2 of an endogenous CD33 gene, wherein the alteration results in a reduced expression level of an epitope encoded by exon 2 of CD33 that is less than 20% of the level as compared with a wild-type counterpart cell.
12. A genetically engineered hematopoietic stem or progenitor cell, which comprises an altered splice acceptor or exonic splicing enhancer site in the exon 2 of an endogenous CD33 gene, wherein the alteration is a nucleotide substitution in the splice acceptor or exonic splicing enhancer site in exon 2 of CD33.
13. A genetically engineered hematopoietic stem or progenitor cell, which comprises an altered splice acceptor or exonic splicing enhancer site in the exon 2 of an endogenous CD33 gene, wherein the alteration is a nucleotide substitution in the nucleotide sequence of the intron 1/exon 2 junction of CD33.
14. A genetically engineered hematopoietic stem or progenitor cell, which comprises an altered splice acceptor or exonic splicing enhancer site in the exon 2 of an endogenous CD33 gene, wherein the alteration is made with the use of a gRNA, which comprises the nucleotide sequence of SEQ ID NO: 1.
15. A genetically engineered hematopoietic stem or progenitor cell, which comprises an altered splice acceptor or exonic splicing enhancer site in the exon 2 of an endogenous CD33 gene, wherein the alteration is made with the use of a gRNA, which comprises the nucleotide sequence of SEQ ID NO: 2.
16. A genetically engineered hematopoietic stem or progenitor cell, which comprises an altered splice acceptor or exonic splicing enhancer site in the exon 2 of an endogenous CD33 gene, wherein the alteration is made with the use of a gRNA, which comprises the nucleotide sequence of SEQ ID NO: 3.
17. The genetically engineered hematopoietic stem or progenitor cell of embodiments 10-16, wherein the alteration is made using a CRISPR-based base editor system.
18. The genetically engineered hematopoietic stem or progenitor cell of embodiments 10-17, wherein the alteration results in a reduced expression level of an epitope encoded by the exon 2 of CD33 as compared with a wild-type counterpart cell (e.g., less than 50%, 40%, 30%, 20%, 15%, 10%, or 5% of the level in the wild-type counterpart cell).
19. The genetically engineered hematopoietic stem or progenitor cell of embodiments 10-18, wherein the reduced expression level of an epitope of CD33 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
20. The genetically engineered hematopoietic stem or progenitor cell of embodiment 19, wherein the cell differentiated from the hematopoietic stem or progenitor cell is a myeloblast, monoblast, monocyte, macrophage, or natural killer cell.
21. A genetically engineered hematopoietic stem or progenitor cell, which comprises an altered splice donor site in the exon 13 of an endogenous EMR2 gene, wherein the alteration results in a reduced expression level of an epitope encoded by exon 13 of EMR2 as compared with a wild-type counterpart cell.
22. A genetically engineered hematopoietic stem or progenitor cell, which comprises an altered splice donor site in the exon 13 of an endogenous EMR2 gene, wherein the altered splice donor site induces an early codon termination and production of a mutated or truncated EMR2 as compared with a wildtype counterpart.
23. A genetically engineered hematopoietic stem or progenitor cell, which comprises an altered splice donor site in the exon 13 of an endogenous EMR2 gene, wherein the alteration is a nucleotide substitution in the splice acceptor or exonic splicing enhancer site in exon 13 of EMR2.
24. A genetically engineered hematopoietic stem or progenitor cell, which comprises an altered splice donor site in the exon 13 of an endogenous EMR2 gene, wherein the alteration is a nucleotide substitution in the nucleotide sequence of the intron 12/exon 13 junction of EMR2.
25. A genetically engineered hematopoietic stem or progenitor cell, which comprises an altered splice donor site in the exon 13 of an endogenous EMR2 gene, wherein the alteration is made with the use of a gRNA, which comprises the nucleotide sequence of any one of SEQ ID NOs: 4 or 46-47.
26. The genetically engineered hematopoietic stem or progenitor cell of embodiments 21-25, wherein the alteration is made using a CRISPR-based base editor system.
27. The genetically engineered hematopoietic stem or progenitor cell of embodiments 21-25, wherein the alteration results in a reduced expression level of an epitope encoded by the exon 13 of EMR2 as compared with a wild-type counterpart cell (e.g., less than 50%, 40%, 30%, 20%, 15%, 10%, or 5% of the level in the wild-type counterpart cell).
28. The genetically engineered hematopoietic stem or progenitor cell of embodiments 21-26, wherein the reduced expression level of an epitope of EMR2 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
29. The genetically engineered hematopoietic stem or progenitor cell of embodiment 29, wherein the cell differentiated from the hematopoietic stem or progenitor cell is a myeloblast, monoblast, monocyte, macrophage, or natural killer cell.
30. The genetically engineered hematopoietic stem or progenitor cell of any of the preceding embodiments, which is CD34+.
31. The genetically engineered hematopoietic stem or progenitor cell of any of the preceding embodiments, which is from bone marrow cells or peripheral blood mononuclear cells of a subject.
32. The genetically engineered hematopoietic stem or progenitor cell of embodiment 31, wherein the subject is a human patient having a hematopoietic malignancy.
33. The genetically engineered hematopoietic stem or progenitor cell of embodiment 31, wherein the subject is a healthy human donor (e.g., an HLA-matched donor).
34. A genetically engineered hematopoietic stem or progenitor cell, which comprises at least one nucleotide substitution in a gene encoding a lineage-specific antigen, wherein the nucleotide substitution is comprised within a sequence encoding a splice element and results in an alternative splicing of a transcript encoded by the gene, wherein the alternative splicing causes a reduced expression level of an epitope encoded by the gene as compared with a wild-type counterpart cell, and wherein the epitope is targeted by an immunotherapeutic agent, and at least one nucleotide substitution in a gene encoding at least one additional lineage-specific antigen, wherein the nucleotide substitution is comprised within a sequence encoding a splice element and results in an alternative splicing of a transcript encoded by the gene, wherein the alternative splicing causes a reduced expression level of an epitope encoded by the gene encoding at least one additional lineage-specific antigen as compared with a wild-type counterpart, and wherein both epitopes are targeted by an immunotherapeutic agent(s).
35. The genetically engineered hematopoietic stem or progenitor cell of embodiment 34, wherein the splice element is chosen from the group consisting of a splice acceptor, splice donor, splice enhancer and splice silencer.
36. The genetically engineered hematopoietic stem or progenitor cell of embodiments 34-35, wherein alternative splicing causes an exon encoding an epitope to be skipped.
37. The genetically engineered hematopoietic stem or progenitor cell of embodiments 34-35, wherein alternative splicing causes an exon encoding an epitope to be extended.
38. The genetically engineered hematopoietic stem or progenitor cell of embodiments 34-37, wherein the nucleotide substitution is a “C” to an “T” or a “G” to an “A”.
39. The genetically engineered hematopoietic stem or progenitor cell of embodiments 34-37, wherein the nucleotide substitution is a “A” to an “G” or a “T” to a “C”.
40. The genetically engineered hematopoietic stem or progenitor cell of embodiments 34-39, wherein the nucleotide substitution is made using a CRISPR-based base editor system.
41. The genetically engineered hematopoietic stem or progenitor cell of embodiments 34-40, wherein the reduced expression level of the epitope is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
42. The genetically engineered hematopoietic stem or progenitor cell of embodiment 41, wherein the cell differentiated from the hematopoietic stem or progenitor cell is a myeloblast, monoblast, monocyte, macrophage, or natural killer cell.
43. A genetically engineered hematopoietic stem or progenitor cell, which comprises an altered splice acceptor or exonic splicing enhancer site in the exon 2 of an endogenous CD33 gene, wherein the alteration is a nucleotide substitution in the splice acceptor or exonic splicing enhancer site in exon 2 of CD33 and an alteration of an altered element in an exon of an endogenous gene encoding at least one additional lineage-specific cell-surface antigen.
44. A genetically engineered hematopoietic stem or progenitor cell, which comprises an altered splice acceptor or exonic splicing enhancer site in the exon 2 of an endogenous CD33 gene, wherein the alteration is a nucleotide substitution in the splice acceptor or exonic splicing enhancer site in exon 2 of CD33 and an altered splice donor site in the exon 13 of an endogenous EMR2 gene, wherein the alteration is a nucleotide substitution in the splice donor site in exon 13 of EMR2.
45. A genetically engineered hematopoietic stem or progenitor cell, comprising an altered splice acceptor or exonic splicing enhancer site in the exon 2 of an endogenous CD33 gene, wherein the alteration is a nucleotide substitution in the splice acceptor or exonic splicing enhancer site in exon 2 of CD33 and results in a reduced expression level of an epitope encoded by exon 2 of CD33 as compared with a wild-type counterpart cell and further comprising an altered splice donor site in the exon 13 of an endogenous EMR2 gene, wherein the alteration is a nucleotide substitution in the splice donor site in exon 13 of EMR2 and results in a reduced expression level of an epitope encoded by exon 13 of EMR2 as compared with a wild-type counterpart cell and/or induces an early codon termination and production of a mutated or truncated EMR2 as compared with a wildtype counterpart cell.
46. A genetically engineered hematopoietic stem or progenitor cell, comprising an altered splice acceptor or exonic splicing enhancer site in the exon 2 of an endogenous CD33 gene, wherein the alteration is a nucleotide substitution in the splice acceptor or exonic splicing enhancer site in exon 2 of CD33 and is made by the use of a gRNA, which comprises the nucleotide sequence of SEQ ID NO: 1, and further comprising an altered splice donor site in exon 13 of an endogenous EMR2 gene, wherein the alteration is a nucleotide substitution in the splice donor site in exon 13 of EMR2 and is made with the use of a gRNA, which comprises the nucleotide sequence of any one of SEQ ID NOs: 4 or 46-47.
47. A genetically engineered hematopoietic stem or progenitor cell, comprising an altered splice acceptor or exonic splicing enhancer site in the exon 2 of an endogenous CD33 gene, wherein the alteration is a nucleotide substitution in the splice acceptor or exonic splicing enhancer site in exon 2 of CD33 and is made by the use of a gRNA, which comprises the nucleotide sequence of SEQ ID NO: 2, and further comprising an altered splice donor site of exon 13 of an endogenous EMR2 gene, wherein the alteration is a nucleotide substitution in the splice donor site in exon 13 of EMR2 and is made with the use of a gRNA, which comprises the nucleotide sequence of any one of SEQ ID NOs: 4 or 46-47.
48. A genetically engineered hematopoietic stem or progenitor cell, comprising an altered splice acceptor or exonic splicing enhancer site in the exon 2 of an endogenous CD33 gene, wherein the alteration is a nucleotide substitution in the splice acceptor or exonic splicing enhancer site in exon 2 of CD33 and is made by the use of a gRNA, which comprises the nucleotide sequence of SEQ ID NO: 3, and further comprising an altered splice donor site of exon 13 of an endogenous EMR2 gene, wherein the alteration is a nucleotide substitution in the splice donor site in exon 13 of EMR2 and is made with the use of a gRNA, which comprises the nucleotide sequence of any one of SEQ ID NOs: 4 or 46-47.
49. The genetically engineered hematopoietic stem or progenitor cell of embodiments 43-48, wherein the alteration(s) is made using a CRISPR-based base editor system.
50. The genetically engineered hematopoietic stem or progenitor cell of embodiments 43-49, wherein the alteration results in a reduced expression level of an epitope encoded by the exon 2 of CD33 as compared with a wild-type counterpart cell (e.g., less than 50%, 40%, 30%, 20%, 15%, 10%, or 5% of the level in the wild-type counterpart cell) and/or a reduced expression level of an epitope encoded by the exon 13 of EMR2 as compared with a wild-type counterpart cell (e.g., less than 50%, 40%, 30%, 20%, 15%, 10%, or 5% of the level in the wild-type counterpart cell).
51. The genetically engineered hematopoietic stem or progenitor cell of embodiments 43-50, which expresses less than 20% of an epitope encoded by the exon 2 of the CD33 expressed by the wild-type counterpart and/or less than 20% of an epitope encoded by the exon 13 of EMR2 expressed by the wild-type counterpart.
52. The genetically engineered hematopoietic stem or progenitor cell of embodiments 43-51, wherein the reduced expression level of an epitope of CD33 and/or EMR2 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
53. The genetically engineered hematopoietic stem or progenitor cell of embodiment 52, wherein the cell differentiated from the hematopoietic stem or progenitor cell is a myeloblast, monoblast, monocyte, macrophage, or natural killer cell.
54. The genetically engineered hematopoietic stem or progenitor cell of embodiments 34-53, which is CD34+.
55. The genetically engineered hematopoietic stem or progenitor cell of embodiments 34-53, which is from bone marrow cells or peripheral blood mononuclear cells of a subject.
56. The genetically engineered hematopoietic stem or progenitor cell of embodiment 55, wherein the subject is a human patient having a hematopoietic malignancy.
57. The genetically engineered hematopoietic stem or progenitor cell of embodiment 55, wherein the subject is a healthy human donor (e.g., an HLA-matched donor).
58. The genetically engineered hematopoietic stem or progenitor cell of embodiments 43-57, which was made by a process comprising contacting the endogenous CD33 gene with a catalytically impaired CRISPR endonuclease fused to a cytosine or adenosine deaminase (base editor) and contacting the endogenous EMR2 gene with a catalytically impaired CRISPR endonuclease fused to a cytosine or adenosine deaminase (base editor).
59. The genetically engineered hematopoietic stem or progenitor cell of any of the preceding embodiments, which does not comprise a mutation in any predicted off-target site.
60. A cell population, comprising a plurality of the genetically engineered hematopoietic stem or progenitor cells of any of the preceding embodiments (e.g., comprising hematopoietic stem cells, hematopoietic progenitor cells, or a combination thereof).
61. A cell population comprising a plurality of genetically engineered hematopoietic stem or progenitor cells which comprise a deletion or alteration of exon 2 of an endogenous CD33 gene and a deletion or alteration of exon 13 of an endogenous EMR2 gene.
62. The cell population of embodiment 61, which further comprises one or more cells that comprise one or more non-engineered CD33 genes and/or non-engineered EMR2 genes.
63. The cell population of embodiments 61-62, which further comprises one or more cells that are homozygous wild-type for CD33 and/or are homozygous wild-type for EMR2.
64. The cell population of any of embodiments 61-63 which further comprises one or more cells that are heterozygous wild-type for CD33 and/or heterozygous wild-type of EMR2.
65. The cell population of any of embodiments 61-64, wherein the reduced expression level of CD33 and/or EMR2 is in a cell differentiated from (e.g., terminally differentiated from) the hematopoietic stem or progenitor cell, and the wild-type counterpart cell is a cell differentiated from (e.g., terminally differentiated from) a wild-type hematopoietic stem or progenitor cell.
66. The cell population of embodiment 65, wherein the cell differentiated from the hematopoietic stem or progenitor cell is a myeloblast, monoblast, monocyte, macrophage, or natural killer cell.
67. The cell population of any of embodiments 61-66, which comprises hematopoietic stem cells and hematopoietic progenitor cells.
68. A pharmaceutical composition comprising the genetically engineered hematopoietic stem or progenitor cell of any of embodiments 1-59.
69. A pharmaceutical composition comprising the cell population of any of embodiments 60-67.
70. A method of making the genetically engineered hematopoietic stem or progenitor cell of any of embodiments 1-33, or the cell population of any of embodiments 60-67, which comprises:
   (i) providing a hematopoietic stem or progenitor cell (e.g., a wild-type hematopoietic stem or progenitor cell), and
   (ii) introducing into the cell (a) a guide RNA (gRNA) that comprises a targeting domain targeting a nucleotide sequence within the genome of the hematopoietic stem or progenitor cell that comprises a splice element, and (b) a catalytically impaired Cas protein fused to a DNA-modifying enzyme fused to a cytosine or adenosine deaminase (base editor), thereby producing a genetically engineered hematopoietic stem and/or progenitor cell.
71. A method of making the genetically engineered hematopoietic stem or progenitor cell of any of embodiments 34-59, or the cell population of any of embodiments 60-67, which comprises:
   (i) providing a hematopoietic stem or progenitor cell, and
   (ii) introducing into the cell (a) a first guide RNA (gRNA) that comprises a targeting domain targeting a first nucleotide sequence within the genome of the hematopoietic stem or progenitor cell that comprises a splice element; and (b) a first catalytically impaired Cas9 endonuclease fused to a cytosine or adenosine deaminase (base editor) and
   (iii) further introducing into the cell (a) a second guide RNA (gRNA) that comprises a targeting domain targeting a second nucleotide sequence within the genome of the hematopoietic stem or progenitor cell that comprises a splice element; and (b) a second catalytically impaired Cas9 endonuclease) fused to cytosine or adenosine deaminase (base editor), hereby producing a genetically engineered hematopoietic stem or progenitor cell.
72. The method of embodiments 70-71, wherein the method results in a nucleotide substitution in an endogenous gene(s) encoding the lineage-specific cell-surface antigen(s).
73. The method of any of embodiments 70-72, wherein the method targets a splice element in an endogenous gene(s), wherein the method results in an alternative splicing of a transcript encoded by the gene(s).
74. The method of any of embodiments 70-73, wherein the alternative splicing causes an exon encoding an epitope to be skipped.
75. The method of any of embodiments 70-73, wherein the alternative splicing causes an exon encoding an epitope to be extended.
76. The method of any of embodiments 70-75, wherein the splice element(s) is a splice donor, a splice acceptor, a splice enhancer or a splice silencer.
77. The method of any of embodiments 70-76, wherein the base editor(s) is a cytosine base editor.
78. The method of any of embodiments 70-76, wherein the base editor(s) is an adenine base editor.
79. The method of any of embodiments 70-78, wherein the endonuclease(s) is a Cas9 nickase.
80. The method of embodiment 77, wherein the cytosine base editor(s) is BE4Max.
81. The method of embodiment 78, wherein the adenosine base editor(s) is ABE8e.
82. The method of any of embodiments 70-81, wherein the alternative splicing results in reduced expression of an epitope encoded by exon 2 of CD33.
83. The method of any of embodiments 70-81, wherein the alternative splicing results in reduced expression of an epitope encoded by exon 13 of EMR2 and/or an early codon termination and production of a mutated or truncated EMR2 as compared with a wildtype counterpart cell.
84. A guide ribonucleic acid (gRNA) comprising a sequence of any of SEQ ID NOs: 1-4 and 46-47, or the reverse complement thereof, or a sequence having at least 90% or 95% identity to any of the foregoing, or a sequence having no more than 1, 2, or 3 mutations relative to any of the foregoing.
85. The gRNA of embodiment 84, which comprises one or more chemical modifications (e.g., a chemical modification to a nucleobase, sugar, or backbone portion).
86. The gRNA of any of embodiments 84 and 85, which binds Cas9.
87. A kit or composition comprising a gRNA chosen from the group consisting of gRNAs comprising SEQ ID NOs: 1-3 and combinations thereof, or a nucleic acid encoding the gRNA.
88. The kit of embodiment 87, further comprising a second gRNA, or a nucleic acid encoding the second gRNA, wherein the second gRNA targets a lineage-specific cell-surface antigen other than CD33.
89. The kit or composition of embodiment 88, wherein the second gRNA targets EMR2.
90. The kit or composition of any of embodiments 89 and 90, wherein the second gRNA comprises any one of SEQ ID NOs: 4 or 46-47.
91. The kit or composition of any of embodiments 88-90, which further comprises a third gRNA, or a nucleic acid encoding the third gRNA.
92. The kit or composition of embodiment 91, wherein the third gRNA targets a lineage-specific cell-surface antigen, other than CD33 and EMR2.
93. The kit or composition of any of embodiments 87-92, further comprising one or more catalytically impaired CRISPR endonucleases fused to a cytosine or adenosine deaminase (base editor).
94. Use of a CRISPR-based base editor system for reducing expression of CD33 in a sample of hematopoietic cells stem or progenitor cells, wherein the gRNA comprises SEQ ID NOs: 1-3.
95. Use of a CRISPR-based base editor system for reducing expression of EMR2 in a sample of hematopoietic cells stem or progenitor cells, wherein the gRNA comprises SEQ ID NOs: 4 and 46-47.
96. Use of a CRISPR-based base editor system for reducing expression of CD33 and/or EMRR2 in a sample of hematopoietic cells stem or progenitor cells, wherein the gRNA comprises SEQ ID NOs: 1-4 and 46-47.
97. A method of producing a genetically engineered hematopoietic stem or progenitor cell, comprising:
   (i) providing a hematopoietic stem or progenitor cell, and
   (ii) introducing into the cell (a) a guide RNA (gRNA) comprising SEQ ID NOs: 1-3; and (b) a nuclease (e.g., an endonuclease) that binds the gRNA fused to cytosine or adenosine deaminase (base editor), thereby producing a genetically engineered hematopoietic stem or progenitor cell.
98. A method of producing a genetically engineered hematopoietic stem or progenitor cell, comprising:
   (i) providing a hematopoietic stem or progenitor cell, and
   (ii) introducing into the cell (a) a guide RNA (gRNA) that comprises a nucleotide sequence at least 90% identical to SEQ ID NOs: 1-3; and (b) a Cas9 endonuclease fused to cytosine or adenosine deaminase (base editor), thereby producing the genetically engineered hematopoietic stem or progenitor cell.
99. The method of embodiments 97 and 98, wherein the gRNA sequence targets the intron 1/exon 2 junction of CD33.
100. The method of embodiments 97-99, wherein the gRNA targets a nucleotide sequence comprising SEQ ID NO: 37.
101. The method of any of embodiments 97-100, wherein the method results in a nucleotide substitution within a sequence encoding a splice element of exon 2 of CD33 and wherein the nucleotide substitution results in an alternative splicing of a transcript encoded by the gene.
102. The method of embodiment 101, wherein the alternative splicing of the transcript results in reduced expression of an epitope of exon 2 of CD33.
103. The method of any of embodiments 97-102, wherein the base editor is a cytosine base editor and is BE4max.
104. The method of any of embodiments 97-102, wherein the base editor is an adenosine base editor and is ABE8e.
105. The method of any of embodiments 97-104, which results in the genetically engineered hematopoietic stem or progenitor cell having a reduced expression level of an epitope of exon 2 of CD33 as compared with a wild-type counterpart cell.
106. The method of any of embodiments 97-105, which is performed on a plurality of hematopoietic stem or progenitor cells.
107. The method of any of embodiments 97-106, which produces a cell population according to any of embodiments 60-67.
108. A method of producing a genetically engineered hematopoietic stem or progenitor cell, comprising:
   (i) providing a hematopoietic stem or progenitor cell, and
   (ii) introducing into the cell (a) a guide RNA (gRNA) comprising SEQ ID NOs: 4 or 46-47; and (b) a nuclease (e.g., an endonuclease) that binds the gRNA fused to cytosine or adenosine deaminase (base editor), thereby producing a genetically engineered hematopoietic stem or progenitor cell.
109. A method of producing a genetically engineered hematopoietic stem or progenitor cell, comprising:
   (i) providing a hematopoietic stem or progenitor cell, and
   (ii) introducing into the cell (a) a guide RNA (gRNA) that comprises a nucleotide sequence at least 90% identical to SEQ ID NOs: 4 or 46-47; and (b) a Cas9 endonuclease fused to cytosine or adenosine deaminase (base editor), thereby producing the genetically engineered hematopoietic stem or progenitor cell.
110. The method of embodiments 108 and 109, wherein the gRNA sequence targets the intron 12/exon 13 junction of EMR2.
111. The method of embodiments 108-110, wherein the gRNA targets a nucleotide sequence comprising SEQ ID NO: 40.
112. The method of any of embodiments 108-111, wherein the method results in a nucleotide substitution in within a sequence encoding a splice element of exon 13 of EMR2 and wherein the nucleotide substitution results in an alternative splicing of a transcript encoded by the gene.
113. The method of embodiment 112, wherein the alternative splicing of the transcript results in reduced expression of an epitope of exon 13 of EMR2 and/or induces an early codon termination and production of a mutated or truncated EMR2 as compared with a wildtype counterpart
114. The method of any of embodiments 108-112, wherein the base editor is a cytosine base editor and is BE4max.
115. The method of any of embodiments 108-112, wherein the base editor is an adenosine base editor and is ABE8e.
116. The method of any of embodiments 108-115, which results in the genetically engineered hematopoietic stem or progenitor cell having a reduced expression level of an epitope of exon 13 of EMR2 as compared with a wild-type counterpart cell.
117. The method of any of embodiments 108-116, which is performed on a plurality of hematopoietic stem or progenitor cells.
118. The method of any of embodiments 108-117, which produces a cell population according to any of embodiments 60-67.
119. A method of producing a genetically engineered hematopoietic stem or progenitor cell, comprising:
   (i) providing a hematopoietic stem or progenitor cell (e.g., a wild-type hematopoietic stem or progenitor cell),
   (ii) introducing into the cell (a) a guide RNA (gRNA) comprising SEQ ID NOs: 1-3; and (b) a nuclease (e.g., an endonuclease) that binds the gRNA (e.g., a Cas9 endonuclease) fused to cytosine or adenosine deaminase (base editor), and
   (iii) further introducing into the cells (a) a guide RNA (gRNA) which targets an at least one additional lineage-specific cell-surface antigen; and (b) a nuclease (e.g., an endonuclease) that binds the gRNA (e.g., a Cas9 endonuclease) fused to cytosine or adenosine deaminase (base editor), thereby producing a genetically engineered hematopoietic stem or progenitor cell.
120. A method of producing a genetically engineered hematopoietic stem or progenitor cell, comprising:
   (i) providing a genetically engineered hematopoietic stem and/or progenitor cell,
   (ii) introducing into the cell (a) a guide RNA (gRNA) that comprises a nucleotide sequence at least 90% identical to SEQ ID NO: 1-3; and (b) a Cas9 endonuclease fused to cytosine or adenosine deaminase (base editor), and
   (iii) further introducing into the cells (a) a guide RNA (gRNA) which targets an at least one additional lineage-specific cell-surface antigen; and (b) a Cas9 endonuclease fused to cytosine or adenosine deaminase (base editor), thereby producing a genetically engineered hematopoietic stem and/or progenitor.
121. The method of any of embodiments 119-120, which results in the genetically engineered hematopoietic stem or progenitor cell having a reduced expression level of an epitope of exon 2 of CD33 and/or a reduced expression level of an epitope of an exon of the at least one additional lineage-specific cell-surface antigen as compared with a wild-type counterpart cell.
122. The method of any of embodiments 119-121, which is performed on a plurality of hematopoietic stem or progenitor cells.
123. The method or use of any of embodiments 119-122, which is performed on a cell population comprising a plurality of hematopoietic stem cells and a plurality of hematopoietic progenitor cells.
124. The method of any of embodiments 119-123, which produces a cell population according to any of embodiments 60-67.
125. The method of any of embodiments 119-123, wherein the at least one additional lineage-specific cell-surface antigen is EMR2 and the method results in a reduced expression level of an epitope encoded by exon 13 of EMR2 and/or induces an early codon termination and production of a mutated or truncated EMR2 as compared with a wildtype counterpart.
126. The method of embodiment 125, wherein the gRNA is SEQ ID NOs: 4 or 46-47.
127. The method of any of embodiments 97-126, wherein the nucleic acids of (a) and (b) are encoded on one vector, which is introduced into the cell.
128. The method of embodiment 127, wherein the vector is a viral vector.
129. The method of any of embodiments 97-126, wherein the base editor is in the form of a protein and (a) and (b) are introduced into the cell as a pre-formed ribonucleoprotein complex.
130. The method of embodiment 129, wherein the ribonucleoprotein complex is introduced into the cell via electroporation.
131. The method of any of embodiments 97-130, wherein the gRNA is a single-molecule guide RNA (sgRNA).
132. The method of any of embodiments 97-131, wherein the gRNA is a modified sgRNA.
133. The method of any of embodiments 97-132, wherein the gRNA is a chemically modified sgRNA.
134. The method of any of embodiments 97-133, wherein the hematopoietic stem or progenitor cell is CD34+.
135. The method of any of embodiments 97-133, wherein the hematopoietic stem or progenitor cell is from bone marrow cells or peripheral blood mononuclear cells (PBMCs) of a subject.
136. The method of embodiment 135, wherein the subject has a hematopoietic disorder.
137. The method of embodiment 135, wherein the subject is a healthy donor.
138. A genetically engineered hematopoietic stem or progenitor cell, which is produced by a method or use of any of embodiments 97-137.
139. A cell population, comprising a plurality of the genetically engineered hematopoietic stem or progenitor cells of embodiment 138 (e.g., comprising hematopoietic stem cells, hematopoietic progenitor cells, or a combination thereof).
140. A method of treating a hematopoietic disorder, comprising administering to a subject in need thereof an effective amount of the genetically engineered hematopoietic stem or progenitor cell of any of embodiments 1-43 and 138 or the cell population of any of embodiments 60-67 and 169.
141. A genetically engineered hematopoietic stem or progenitor cell of any of embodiments 1-20 and 138 or the cell population of any of embodiments 60-67 and 169, for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets CD33, wherein the agent comprises an antigen-binding fragment that binds CD33.
142. A combination of a genetically engineered hematopoietic stem or progenitor cell of any of embodiments 1-20 and 138 or a cell population of any of embodiments 60-67 and 169, and an agent that targets CD33, wherein the agent comprises an antigen-binding fragment that binds CD33, for use in treating a hematopoietic disorder, wherein the treating comprises administering to a patient in need thereof an effective amount of the genetically engineered hematopoietic stem or progenitor cell or the cell population, and the agent that binds CD33.
143. A genetically engineered hematopoietic stem or progenitor cell of any of embodiments 1-9, 21-29 and 138 or the cell population of any of embodiments 60-67 and 169, for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets EMR2, wherein the agent comprises an antigen-binding fragment that binds EMR2.
144. A combination of a genetically engineered hematopoietic stem or progenitor cell of any of embodiments 1-9, 21-29 and 138 or a cell population of any of embodiments 60-67 and 169, and an agent that targets EMR2, wherein the agent comprises an antigen-binding fragment that binds EMR2, for use in treating a hematopoietic disorder, wherein the treating comprises administering to a patient in need thereof an effective amount of the genetically engineered hematopoietic stem or progenitor cell or the cell population, and the agent that binds EMR2.
145. A genetically engineered hematopoietic stem or progenitor cell of any of embodiments 1-9, 43-59 and 138 or the cell population of any of embodiments 60-67 and 169, for use in treating a hematopoietic disorder, wherein the treating comprises administering to a subject in need thereof an effective amount of the genetically engineered hematopoietic stem or progenitor cell or the cell population, and further comprises administering to the subject an effective amount of an agent that targets CD33, wherein the agent comprises an antigen-binding fragment that binds CD33 and an effective amount of an agent that targets an at least one additional lineage-specific cell-surface antigen, wherein the agent comprises an antigen-binding fragment that binds the at least one additional lineage-specific cell-surface antigen.
146. A combination of a genetically engineered hematopoietic stem or progenitor cell of any of embodiments 1-9, 43-59 and 138 or a cell population of any of embodiments 60-67 and 169, and an agent that targets CD33, wherein the agent comprises an antigen-binding fragment that binds CD33 and an agent that targets an at least one additional lineage-specific cell-surface antigen, wherein the agent comprises an antigen-binding fragment that binds at least one additional lineage-specific cell-surface antigen, for use in treating a hematopoietic disorder, wherein the treating comprises administering to a patient in need thereof an effective amount of the genetically engineered hematopoietic stem or progenitor cell or the cell population, and the agent that binds CD33 and the at least one additional lineage-specific cell-surface antigen.
147. The combination of embodiment 145 and the genetically engineered hematopoietic stem or progenitor cell of embodiment 146, wherein the at least one additional lineage-specific cell-surface antigen is EMR2.
148. A genetically engineered hematopoietic stem or progenitor cell of any of embodiments 1-59 and 138, or a cell population of any of embodiments 60-67 and 169 for use in cancer immunotherapy.
149. A genetically engineered hematopoietic stem or progenitor cell of any of embodiments 1-59 and 138, or a cell population of any of embodiments 60-67 and 169 for use in cancer immunotherapy, wherein the patient has a hematopoietic disorder.
150. A genetically engineered hematopoietic stem or progenitor cell of any of embodiments 1-59 and 138, or a cell population of any of embodiments 60-67 and 169 for use in hematopoietic repopulation of a patient having a hematopoietic disorder.
151. A genetically engineered hematopoietic stem or progenitor cell of any of embodiments 1-59 and 138, or a cell population of any of embodiments 60-67 and 169 for use in a method of treating a hematopoietic disorder, whereby the genetically engineered hematopoietic stem or progenitor cell described herein or a cell population described herein repopulate the patient.
152. A genetically engineered hematopoietic stem or progenitor cell of any of embodiments 1-59 and 138, or a cell population of any of embodiments 60-67 and 169 for use in reducing cytotoxic effects of an agent that targets CD33 and/or an at least one additional lineage-specific cell-surface antigen in immunotherapy.
153. A genetically engineered hematopoietic stem or progenitor cell of any of embodiments 1-59 and 138, or a cell population of any of embodiments 60-67 and 169 for use in an immunotherapy method using an agent that targets CD33 and/or at least one additional lineage-specific cell-surface antigen, whereby the genetically engineered hematopoietic stem or progenitor cell described herein or a cell population described herein reduces cytotoxic effects of the agent that targets CD33 and/or at least one additional lineage-specific cell-surface antigen.
154. The method, cell, agent, or combination of any of embodiments 140-153, wherein the genetically engineered hematopoietic stem or progenitor cell or the cell population is administered concomitantly with the agent that targets CD33 and/or at least one additional lineage-specific cell-surface antigen.
155. The method, cell, agent, or combination of any of embodiments 140-153, wherein the genetically engineered hematopoietic stem or progenitor cell or the cell population is administered prior to the agent that targets CD33 and/or an at least one additional lineage-specific cell-surface antigen.
156. The method, cell, agent, or combination of any of embodiments 140-153, wherein the agent that targets CD33 and/or an at least one additional lineage-specific cell-surface antigen is administered prior to the genetically engineered hematopoietic stem or progenitor cell or the cell population.
157. The method, cell, agent, use, or combination of any of embodiments 140-156, wherein the hematopoietic disorder is a hematopoietic malignancy.
158. The method, cell, agent, use, or combination of any of embodiments 140-157, further comprising administering to the subject an effective amount of an agent that targets CD33, and wherein the agent comprises an antigen-binding fragment that binds CD33 and/or an effective amount of an agent that targets an at least one additional lineage-specific cell-surface antigen, and wherein the agent comprises an antigen-binding fragment that binds the at least one additional lineage-specific cell-surface antigen.
159. The method, cell, agent, use, or combination of embodiment 158, wherein the agent that targets CD33 is an immune cell expressing a chimeric antigen receptor (CAR), which comprises the antigen-binding fragment that binds CD33 and the agent that targets the at least one additional lineage-specific cell-surface antigen is an immune cell expressing a chimeric antigen receptor (CAR), which comprises the antigen-binding fragment that binds the at least one additional lineage-specific cell-surface antigen.
160. The method, cell, agent, use or combination of embodiment 152-159, wherein the at least one additional lineage-specific cell-surface antigen is EMR2.
161. The method, cell, agent, use, or combination of embodiment 159, wherein the immune cell is a T cell.
162. The method, cell, agent, use, or combination of any of embodiments 140-161, wherein the subject is a human patient having Hodgkin's lymphoma, non-Hodgkin's lymphoma, leukemia, or multiple myeloma.
163. The method, cell, agent, use, or combination of embodiment 163, wherein the subject is a human patient having leukemia, which is acute myeloid leukemia, chronic myelogenous leukemia, acute lymphoblastic leukemia, or chronic lymphoblastic leukemia.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1 presents an exemplary illustration of type 0, type 1, type 2, and type 3 lineage-specific antigens.

FIG. 2 is a schematic showing an immune cell expressing a chimeric receptor that targets the type 0 lineage-specific cell-surface antigen, CD307. Multiple myeloma (MM) cells expressing CD307 as well other cells expressing CD307, such as plasma cells, are targeted by the immune cells expressing the anti-CD307chimeric receptor.

FIG. 3 is a schematic showing an immune cell expressing a chimeric receptor that targets the type 2 lineage-specific cell-surface antigen, CD33. Acute myeloid leukemia (AML) cells expressing CD33. Human hematopoietic stem cells (HSC) are genetically engineered to be deficient in CD33 and therefore not recognized by the immune cells expressing the anti-CD33 chimeric receptor. The HSC are able to give rise to myeloid cells.

FIG. 4 shows the base editing strategy of CD33. FIG. 4A shows the details of exon 1-3 region and possible splicing outcomes, ag: Splicing acceptor site (SA). gt: Splicing donor site. Dotted lines depict possible splicing events. GO Gentuzumab Ozogamicin (Ab icon) recognizes an epitope located in exon 2. FIG. 4B shows Intron1 (lower case)/exon2 (upper case) junction DNA sequence with highlight of exon 2 SA (red) and Exon Splicing Enhancer site (ESE in yellow). FIG. 4C shows CD33 mRNA full length (CD33^{FL}) contains 7 exons. Exon 2 encodes an Ig-like V-type domain. CD33^{Δ2} lacks exon 2 due to a common polymorphism (rs12459419) which changes C>T resulting in an altered exonic splicing enhancer (ESE) site.

FIG. 5 shows that ABE introduce A>G conversions at targeted sites with up to 95% efficiency, negligible indels and induces exon 2 skipping. FIG. 5A are Sanger sequencing profiles of edited cells compared to the wild-type genomic sequence (top). Editing of a cytosine or an adenine that mutates the ESE or SA is indicated by arrows. FIG. 5B shows results of HTS analysis by CRISPResso2 which depicts the editing outcomes at the target site. Approximately 250 bp surrounding the targeted nucleotides was PCR-amplified from extracted genomic DNA and sequenced on an Illumina MiSeq. The results show that each targeted base acquired the intended mutation. FIG. 5C shows assessment of CD33 expression in the edited cells 7 days post electroporation by FACS analysis using 2 different antibodies clones WM53 and P67.6 which recognize an epitope located in exon 2. After BE4-mediated editing, around 30% of the CD34⁺ cells show CD33 expression while less than 5% after ABE-mediated editing. FIG. 5D shows that ESE or SA editing induces exon 2 skipping. Exon skipping in CD34⁺ edited cells was characterized by performing PCR on cDNA with sets of primers, specific to CD33^{Δ2} (spanning exon junction 1-3), or common to all isoforms (in exons 1, 5 and 7). PCR products were separated by polyacrylamide gel electrophoresis and visualized by SYBR-safe fluorescence. Below the gel, Sanger sequencing of PCR products confirm the absence of exon 2 in edited cells while all others exons are intact.

FIG. 6 shows that in vitro differentiated WT or CD33^{Δ2} monocytes display normal phagocytic capacity and that CD34⁺CD33^{Δ2} cells are resistant to GO cytotoxicity in vitro. FIG. 6A shows in vitro differentiated WT or CD33^{Δ2} monocytes show comparable phagocytosis capacity, as measured by E. Coli bioparticles internalization. The left side are representative FACs plots of E. Coli bioparticles internalization by in vitro differentiated WT or CD33^{Δ2}monocytes. Treatment with actin polymerization inhibitor, cytochalasin D abrogates phagocytosis. The right side is a graph illustrating phagocytosis quantification.

FIG. 6B shows that CD34⁺CD33^{Δ2} cells resist GO cytotoxicity in vitro. Cells were incubated 48 hours with GO and cytotoxicity analyzed by FACS. CD34⁺CD33^{Δ2} show same resistance to GO cytotoxicity than a donor with homozygous rs12459419 A14V SNP.

FIG. 7 shows that CD34⁺CD33^{Δ2} engraft, recapitulate a complete hematopoeitic system and are resistant to gemtuzumab ozogamicin (GO) in vivo. FIG. 7A are graphs depicting the frequency of human CD45⁺ cells and frequency of progenitors myeloid (CD123) and lymphoid (CD10), as well as mature myeloid (CD14) and lymphoid (CD19), and T cells (CD3) within the human CD45 population from bone marrow (BM) and spleen analyzed at 16 weeks post-transplantation. FIG. 7B are representative images of H&E staining and immunocytochemistry with anti-CD33 on the BM of mice engrafted with CD34^{+WT} or CD34⁺CD33^{Δ2}. FIG. 7C shows that CD34⁺CD33^{Δ2} cells are resistant to GO in vivo. Peripheral blood (PB) of 12 weeks post-transplanted mice was analyzed for the presence of CD33⁺CD14⁺ cells or CD33^{Δ2}CD14⁺ cells. Mice were then injected with 2.5ugr GO then bled and sacked one week after GO treatment to assess the presence of myeloid cells in the PB and the BM of the humanized mice. Before GO treatment, CD34^{+WT} or CD34⁺CD33^{Δ2} transplanted mice displayed same frequency of CD14⁺ cells in the PB (top FACS plots). One week, after GO injection while CD33⁻CD14⁺ cells are detected in the PB and BM of mice engrafted with CD34⁺CD33^{Δ2} cells, CD33⁺ and CD14⁺ cells have been eradicated in the PB and BM of CD34^{+WT} engrafted mice. FIG. 7D is a graph of the CD33 on-target A-G editing at the target site (A7) in the engrafting WT (unedited) or edited cells from bone marrow samples at 16 weeks post-transplantation. Sixteen weeks after transplantation, the CD33 locus was amplified from genomic DNA from the bone marrow of mice. Amplicons were sequenced by HTS and A-to-G editing at position A7 was quantified

FIG. 8 shows the results of off target analysis. FIG. 8A is a table summarizing the 19 top off target identified loci. FIG. 8B is a graph of the assessment of A-to-G editing at position A7 at the 19 identified top off target loci in engrafting human WT (unedited) or edited cells from bone marrow at 16 weeks post-transplantation. FIG. 8C is a graph of the indels assessment at the 19 identified top off target loci in engrafting human WT (unedited) or edited cells from bone marrow at 16 weeks post-transplantation.

FIG. 9 shows the base editing strategy of CD33 and EMR2 and that ABE introduces A>G conversions at targeted sites which leads to double editing of CD34⁺ cells. FIG. 9A (top) shows details of EMR2, exon 13, gtgagt: Splicing donor site (SD). FIG. 9A (bottom) shows intron12 (lowercase)/exon13 (upper case) junction DNA sequence with highlight of exon 13 SD (red) and in bold the protospacer. FIG. 9B are Sanger sequencing profiles of edited cells are compared to the wild-type genomic sequence (top). Editing of an adenine that mutates the SA of CD33 exon 2 or SD of EMR2 exon 13 is indicated by arrows. FIG. 9C shows FACS analysis of the WT and edited cells one week post nucleofection.

FIGS. 10A-10D show schematics of example chimeric receptors comprising antigen-binding fragments that target CD33. FIG. 10A: a generic chimeric receptor targeting CD33 comprising an anti-CD33 scFv, hinge domain, transmembrane domain, co-stimulatory domain, and signaling domain. FIG. 10B: a chimeric receptor targeting CD33 comprising an anti-CD33 scFv, hinge domain from CD8, transmembrane domain from CD8, and intracellular domains from CD28 and CD3ζ. FIG. 10C: a chimeric receptor targeting CD33 comprising an anti-CD33 scFv, hinge domain from CD8, transmembrane domain from CD8, and intracellular domains from ICOS (or CD27, 4-1BB, or OX-40) and CD3ζ. FIG. 10D: a chimeric receptor targeting CD33 comprising an anti-CD33 scFv, hinge domain from CD8, transmembrane domain from CD8, and intracellular domains from OX40, CD28, and CD3ζ.

FIG. 11 is a schematic of an immunotoxin.

FIGS. 12A-12B show expression of anti-CD33 chimeric receptors expressed in K562 cells transduced with an empty vector or vector encoding an anti-CD33 chimeric receptor. FIG. 12A: Western blot using a primary antibody that recognizes CD3ζ. The table provides the estimated molecular weight of each of the chimeric receptors tested. FIG. 12B: Flow cytometric analysis showing an increase in the population of cells that stain positive for the anti-CD33 chimeric receptor.

FIGS. 13A-13C show the anti-CD33 chimeric receptors bind to CD33. FIG. 13A: Ponceau stained protein gel. Lanes 1, 3, 5: CD33 molecule. Lanes 2, 4, 6: CD33 mol+APC Conjugate. FIG. 13B: Western blot using a primary antibody that recognizes CD3ζ. Lanes 1, 3, and 5 contain the chimeric receptors co-incubated with CD33 molecules, and lanes 2, 4, and 6 contain the chimeric receptors co-incubated with a CD33-APC conjugate. FIG. 13C: Flow cytometric analysis showing an increase in the population of cells that express anti-CD33 chimeric receptors and bind CD33.

FIGS. 14A-14B show cytotoxicity of K562 cells by NK92 cells expressing the indicated chimeric receptors. FIG. 14A: CART1 and CART2 compared to empty HIVzsG vector. FIG. 14B: CART3 compared to empty HIVzsG vector.

FIGS. 15A-15B show cytotoxicity (expressed as percent cytotoxicity on the y-axis) of K562 cells deficient in CD33 by NK92 cells expressing the indicated chimeric receptors. FIG. 15A: unsorted population of K562 cells pretreated with CD33-targeting CRISPR/Cas reagents. FIG. 15B: single clones of K562 cells deficient in CD33. The columns, from left to right, correspond to empty HIVzsG vector, CART1, CART2, and CART3.

FIGS. 16A-16B show flow cytometric analysis of primary T cell populations.

FIG. 16A: sorting of cells based on expression of T cell markers C4⁺, CD8⁺, or both CD4⁺ CD8⁺. FIG. 16B: relative expression of CD33 on the indicated populations of primary T cells.

FIGS. 17A-17B show cytotoxicity of K562 cells by primary T cells expressing the indicated chimeric receptors. FIG. 17A: CD4⁺ T cells. FIG. 17B: CD4⁺/CD8⁺ (CD 4/8) and CD8⁺ (CD8).

### DETAILED DESCRIPTION OF DISCLOSURE

Cancer immunotherapies targeting antigens present on the cell surface of a cancer cell is particularly challenging when the target antigen is also present on the cell surface of normal, non-cancer cells that are required or critically involved in the development and/or survival of the subject. Targeting these antigens may lead to deleterious effects in the subject due to cytotoxic effects of the immunotherapy toward such cells in addition to the cancer cells.

The methods, nucleic acids, and cells described herein allow for targeting of antigens (e.g., type 1 or type 2 antigens) that are present not only on cancer cells but also cells critical for the development and/or survival of the subject. The method involves: (1) reducing the number of cells carrying the target lineage-specific cell-surface antigen using an agent that targets such an antigen; and (2) replacement of the normal cells (e.g., non-cancer cells) that present the antigen and thus can be killed due to administration of the agent with hematopoietic cells that are deficient for the lineage-specific cell-surface antigen. The methods described herein can maintain surveillance for target cells, including cancer cells that express a lineage-specific cell-surface antigen of interest and also maintain the population of non-cancer cells expressing the lineage-specific antigen, which may be critical for development and/or survival of the subject.

Accordingly, described herein are the co-use of immune cells expressing chimeric receptors comprising an antigen-binding fragment that targets a lineage-specific cell-surface antigen (e.g., CD33) and hematopoietic cells such as hematopoietic stem cells (HSCs) or hematopoietic progenitor cells (HPCs) that are deficient in the lineage-specific cell-surface antigen for treating a hematopoietic malignancy. Also provided herein are the chimeric receptors, nucleic acids encoding such, vectors comprising such, and immune cells (e.g., T cells) expressing such a chimeric receptors.

The present disclosure also provides genetically engineered hematopoietic cells that are deficient in a lineage-specific antigen such as those described herein, as well as methods (e.g., genome editing methods) for making such.

Also described herein are the co-use of immune cells expressing chimeric receptors comprising an antigen-binding fragment that targets a lineage-specific cell-surface antigen (e.g., CD33) and at least one additional lineage-specific cell-surface antigen (e.g., EMR2) and hematopoietic cells such as hematopoietic stem cells (HSCs) or hematopoietic progenitor cells (HPCs) that are deficient in the lineage-specific cell-surface antigen(s) for treating a hematopoietic malignancy. Also provided herein are the chimeric receptors, nucleic acids encoding such, vectors comprising such, and immune cells (e.g., T cells) expressing such a chimeric receptors. The present disclosure also provides genetically engineered hematopoietic cells that are deficient in a lineage-specific antigen such as those described herein, as well as methods (e.g., genome editing methods) for making such.

Also described herein is a method of genome editing hematopoietic cells using a CRISPR-based base editor system. The use of the CIRSPR-based base editor system allows high editing efficiency of HSCs/HSPCs using CRISPR-based cytosine and adenine base editors (CBEs and ABEs). The CBEs and ABEs are Cas9 nickase fused to a cytidine or adenosine deaminase, respectively, enabling precise base substitutions at targeted regions without generating DSBs. Because they elude DSBs, base editors are considered to be safer editing tools that eliminate undesired indels, translocations or rearrangements resulting from DSBs.

Shown herein is the highly effective use of base editors, especially the adenosine base editor, ABE8e to modify hematopoietic cells by specifically altering the nucleotide sequence of a splice element. The alteration of the splice element results in an alternative splicing of a transcript encoded by the gene, further causing a reduced expression level of an epitope encoded by the gene, e.g., encoded by an exon of the gene. Shown herein is the use of base editors and gRNA to alter a splice acceptor or exonic enhancer site in exon 2 of CD33. In particular, the genome editing using the ABE8e base editor and guide RNA specifically designed to target the junction DNA sequence, showed up to 95% efficiency with no indels.

Also shown herein is the use of base editors to alter the splice donor site of exon 13 of EMR2.

Additionally, the Cas9 nickase fused to a cytidine or adenosine deaminase described herein can be used in an mRNA or a protein form. The latter form results in less off target effects than the former and was successfully used to edit HSCs which were able to engraft in vivo and give the CD34 cells resistance to GO.

See, e.g., Examples 1-5.

### Definitions

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. In the context of the present disclosure, the term “subject” also encompasses tissues and cells that can be cultured in vitro or ex vivo or manipulated in vivo. The term “subject” can be used interchangeably with the term “organism”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components. A polynucleotide may also be modified after polymerization, such as by conjugation with a labeling agent.

The term “hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi-stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

The term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the present disclosure are not naturally-occurring as a whole. Parts of the vectors can be naturally-occurring. The non-naturally occurring recombinant expression vectors of the present disclosure can comprise any type of nucleotides, including, but not limited to DNA and RNA, which can be single-stranded or double-stranded, synthesized or obtained in part from natural sources, and which can contain natural, non-natural or altered nucleotides.

“Transfection,” “transformation,” or “transduction,” as used herein, refer to the introduction of one or more exogenous polynucleotides into a host cell by using physical or chemical methods.

“Antibody,” “fragment of an antibody,” “antibody fragment,” “functional fragment of an antibody,” or “antigen-binding portion” are used interchangeably to mean one or more fragments or portions of an antibody that retain the ability to specifically bind to a specific antigen (Holliger et al., Nat. Biotech. (2005) 23(9): 1126). The present antibodies may be antibodies and/or fragments thereof. Antibody fragments include Fab, F(ab′)2, scFv, disulfide linked Fv, Fc, or variants and/or mixtures. The antibodies may be chimeric, humanized, single chain, or bi-specific. All antibody isotypes are encompassed by the present disclosure, including, IgA, IgD, IgE, IgG, and IgM. Suitable IgG subtypes include IgG1, IgG2, IgG3 and IgG4. An antibody light or heavy chain variable region consists of a framework region interrupted by three hypervariable regions, referred to as complementarity determining regions (CDRs). The CDRs of the present antibodies or antigen-binding portions can be from a non-human or a human source. The framework of the present antibodies or antigen-binding portions can be human, humanized, non-human (e.g., a murine framework modified to decrease antigenicity in humans), or a synthetic framework (e.g., a consensus sequence).

The present antibodies or antigen-binding portions can specifically bind with a dissociation constant (K_{D}) of less than about 10⁻⁷ M, less than about 10⁻⁸ M, less than about 10⁻⁹ M, less than about 10⁻¹⁰ M, less than about 10⁻¹¹ M, or less than about 10⁻¹² M. Affinities of the antibodies according to the present disclosure can be readily determined using conventional techniques (see, e.g., Scatchard et al., Ann. N.Y. Acad. Sci. (1949) 51:660; and U.S. Pat. Nos. 5,283,173, 5,468,614, or the equivalent).

The terms “chimeric receptor,” “Chimeric Antigen Receptor,” or alternatively a “CAR” are used interchangeably throughout and refer to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. Lee et al., Clin. Cancer Res. (2012) 18(10):2780; Jensen et al., Immunol Rev. (2014) 257(1):127. In one embodiment, the stimulatory molecule is the zeta chain associated with the T cell receptor complex. In one aspect, the cytoplasmic signaling domain further comprises one or more functional signaling domains derived from at least one costimulatory molecule as defined below. The costimulatory molecule may also be 4-1BB (i.e., CD137), CD27 and/or CD28 or fragments of those molecules. In another aspect, the CAR comprises a chimeric fusion protein comprising an extracellular antigen recognition domain, a transmembrane domain and an intracellular signaling domain comprising a functional signaling domain derived from a stimulatory molecule. The CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. Alternatively, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. The CAR can also comprise a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. The antigen recognition moiety of the CAR encoded by the nucleic acid sequence can contain any lineage specific, antigen-binding antibody fragment. The antibody fragment can comprise one or more CDRs, the variable region (or portions thereof), the constant region (or portions thereof), or combinations of any of the foregoing.

The term “signaling domain” refers to the functional portion of a protein which acts by transmitting information within the cell to regulate cellular activity via defined signaling pathways by generating second messengers or functioning as effectors by responding to such messengers.

The term “zeta” or alternatively “zeta chain”, “CD3-zeta” or “TCR-zeta” is defined as the protein provided as GenBank accession numbers NP_932170, NP_000725, or XP_011508447; or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation.

The term “genetically engineered” or “genetically modified” refers to cells being manipulated by genetic engineering, for example by genome editing. That is, the cells contain a heterologous sequence which does not naturally occur in said cells. Typically, the heterologous sequence is introduced via a vector system or other means for introducing nucleic acid molecules into cells including liposomes. The heterologous nucleic acid molecule may be integrated into the genome of the cells or may be present extra-chromosomally, e.g., in the form of plasmids. The term also includes embodiments of introducing genetically engineered, isolated CAR polypeptides into the cell.

The term “autologous” refers to any material derived from the same individual to whom it is later to be re-introduced into the same individual.

The term “allogeneic” refers to any material derived from a different animal of the same species as the individual to whom the material is introduced. Two or more individuals are said to be allogeneic to one another when the genes at one or more loci are not identical.

The term “cell lineage” refers to cells with a common ancestry and developing from the same type of identifiable cell into specific identifiable/functioning cells. The cell lineages used herein include, but are not limited to, respiratory, prostatic, pancreatic, mammary, renal, intestinal, neural, skeletal, vascular, hepatic, hematopoietic, muscle or cardiac cell lineages.

The term “inhibition” when used in reference to gene expression or function of a lineage specific antigen refers to a decrease in the level of gene expression or function of the lineage specific antigen, where the inhibition is a result of interference with gene expression or function. The inhibition may be complete, in which case there is no detectable expression or function, or it may be partial. Partial inhibition can range from near complete inhibition to a near absence of inhibition. By eliminating particular target cells, CAR T cells may effectively inhibit the overall expression of particular cell lineage.

Cells such as hematopoietic cells that are “deficient in a lineage-specific antigen” refers to cells having a substantially reduced expression level of the lineage-specific antigen as compared with their naturally-occurring counterpart, e.g., endogenous hematopoietic cells of the same type, or cells that do not express the lineage-specific antigen, i.e., not detectable by a routine assay such as FACS. In some instances, the express level of a lineage-specific antigen of cells that are “deficient in the antigen” can be lower than about 40% (e.g., 30%, 20%, 15%, 10%, 5% or lower) of the expression level of the same lineage-specific antigen of the naturally-occurring counterpart.

The term “splice element” as used herein includes splice acceptor sites, splice donor sites, splice enhancer sites, and splice silencer sites.

As used herein, the term “about” refers to a particular value+/−5%. For example, an expression level of about 40% may include any amount of expression between 35%-45%.

### Agents Targeting Lineage-Specific Cell-Surface Antigens

Aspects of the disclosure provide agents (e.g., agents that target CD33, e.g., wherein the agent comprises an antigen-binding fragment that binds CD33 and agents that target at least one additional lineage-specific cell-surface antigen e.g., wherein the agent comprises an antigen-binding fragment that binds at least one additional lineage-specific cell-surface antigen) targeting a lineage-specific cell-surface antigen(s), for example on a target cancer cell. Such an agent may comprise an antigen-binding fragment that binds and targets the lineage-specific cell-surface antigen(s). In some instances, the antigen-binding fragment can be a single chain antibody (scFv) specifically binding to the lineage-specific antigen.

A. Lineage-Specific Cell-Surface Antigens

As used herein, the terms “lineage-specific”, “lineage-specific cell-surface antigen” and “cell-surface lineage-specific antigen” may be used interchangeably and refer to any antigen that is sufficiently present on the surface of a cell and is associated with one or more populations of cell lineage(s). For example, the antigen may be present on one or more populations of cell lineage(s) and absent (or at reduced levels) on the cell-surface of other cell populations.

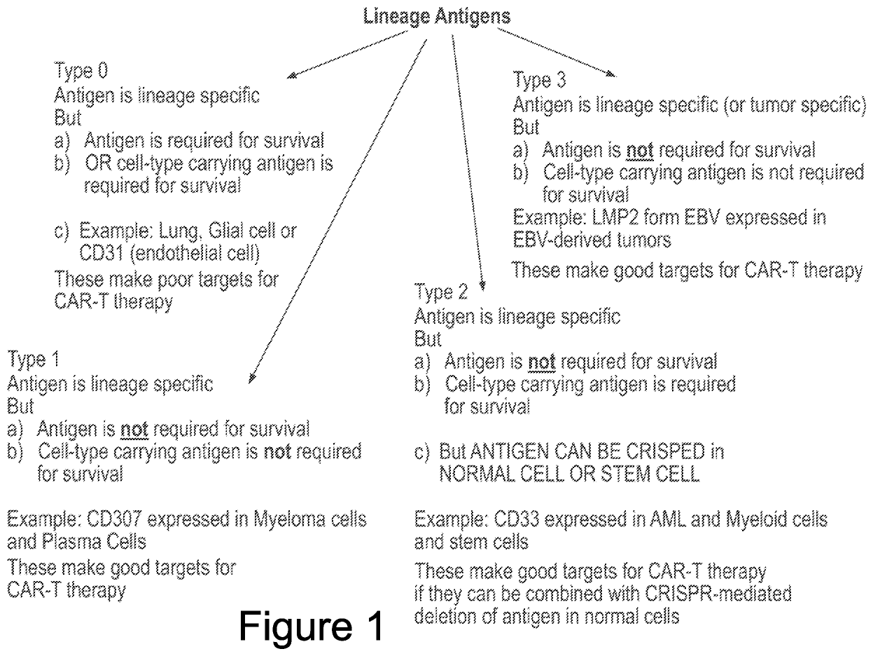

In general, lineage-specific cell-surface antigens can be classified based on a number of factors such as whether the antigen and/or the populations of cells that present the antigen are required for survival and/or development of the host organism. A summary of exemplary types of lineage-specific antigens is provide in Table 1 below. See also FIG. 1.

TABLE 1
Classification of Lineage Specific Antigens

| Type of Lineage | Characteristics of the Lineage |
|---|---|
| Specific Antigen | Specific Antigen |
| Type 0 | a) antigen is required for survival of an |
| | organism |
| | and |
| | b) cell type carrying type 0 antigen is |
| | required for survival of an organism and is |
| | not unique to a tumor, or tumor-associated |
| | virus |
| Type 1 | a) antigen is not required for survival |
| | of an organism |
| | and |
| | b) cell type carrying type 1 antigen is |
| | not required for survival of an organism |
| Type 2 | a) antigen is not required for survival |
| | of an organism |
| | and |
| | b) cell type carrying type 2 antigen is |
| | required for the survival of an organism |
| Type 3 | a) antigen is not required for the |
| | survival of an organism |
| | and |
| | b) cell type carrying antigen is not |
| | required for survival of an organism |
| | c) The antigen is unique to a tumor, or |
| | a tumor associated virus |
| | An example is the LMP-2 antigen in EBV |
| | infected cells, including EBV infected |
| | tumor cells (Nasopharyngeal carcinoma |
| | and Burkitts Lymphoma) |

As shown in Table 1 and FIG. 1, type 0 lineage-specific cell-surface antigens are necessary for the tissue homeostasis and survival, and cell types carrying type 0 lineage-specific cell-surface antigen may be also necessary for survival of the subject. Thus, given the importance of type 0 lineage-specific cell-surface antigens, or cells carrying type 0 lineage-specific cell-surface antigens, in homeostasis and survival, targeting this category of antigens may be challenging using conventional CAR T cell immunotherapies, as the inhibition or removal of such antigens and cell carrying such antigens may be detrimental to the survival of the subject. Consequently, lineage-specific cell-surface antigens (such as type 0 lineage-specific antigens) and/or the cell types that carry such antigens may be required for the survival, for example because it performs a vital non-redundant function in the subject, then this type of lineage specific antigen may be a poor target for CAR T cell based immunotherapy.

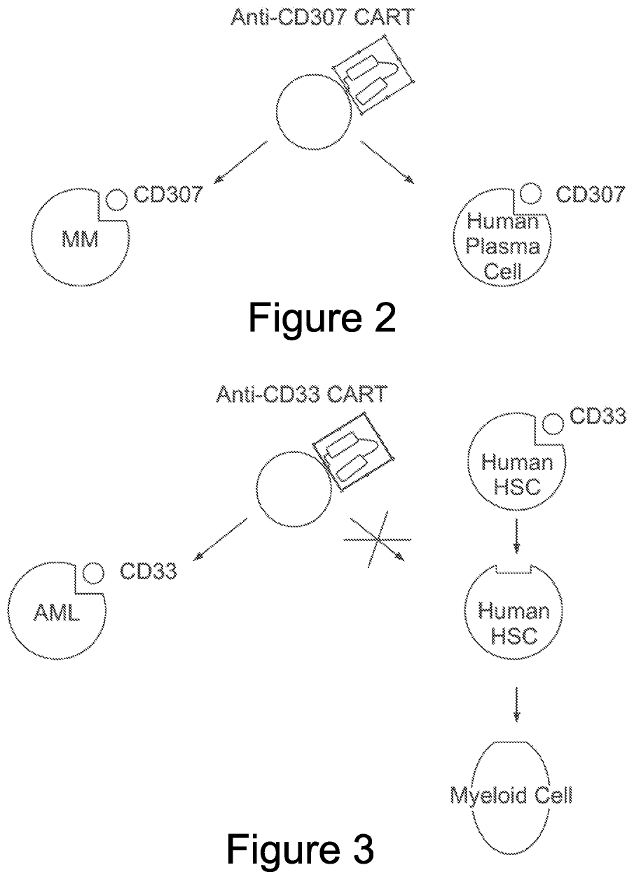

In contrast to type 0 antigens, type 1 lineage-specific cell-surface antigens and cells carrying type 1 lineage-specific cell-surface antigens are not required for tissue homeostasis or survival of the subject. Targeting type 1 lineage-specific cell-surface antigens is not likely to lead to detrimental consequences in the subject. For example, a CAR T cell engineered to target CD307, a type 1 antigen expressed uniquely on both normal plasma cells and multiple myeloma (MM) cells would lead to elimination of both cell types (FIG. 2) (Elkins et al., Mol Cancer Ther. (2012) 10:2222). However, since the plasma cell lineage is expendable for the survival of the organism, CD307 and other type 1 lineage specific antigens are antigens that are suitable for CAR T cell-based immunotherapy. Lineage specific antigens of type 1 class may be expressed in a wide variety of different tissues, including, ovaries, testes, prostate, breast, endometrium, and pancreas. In some embodiments, the agent targets a lineage-specific cell-surface antigen that is a type 1 antigen.

Targeting type 2 antigens presents a significant difficulty as compared to type 1 antigens. Type 2 antigens are those characterized where: (1) the antigen is dispensable for the survival of an organism (i.e., is not required for the survival); and (2) the cell lineage carrying the antigen is indispensable for the survival of an organism (i.e., the particular cell lineage is required for the survival). For example, CD33 is a type 2 antigen expressed in both normal myeloid cells as well as in Acute Myeloid Leukemia (AML) cells (Dohner et al., (2015) NEJM 373:1136). As a result, a CAR T cell engineered to target CD33 antigen could lead to the killing of both normal as well as AML cells, which may be incompatible with survival of the subject (FIG. 3). In some embodiments, the agent targets a lineage-specific antigen cell-surface that is a type 2 antigen.

A wide variety of antigens may be targeted by the methods and compositions of the present disclosure. Monoclonal antibodies to these antigens may be purchased commercially or generated using standard techniques, including immunization of an animal with the antigen of interest followed by conventional monoclonal antibody methodologies e.g., the standard somatic cell hybridization technique of Kohler and Milstein, Nature (1975) 256: 495, as discussed above. The antibodies or nucleic acids encoding for the antibodies may be sequenced using any standard DNA or protein sequencing techniques.

In some embodiments, the lineage-specific cell-surface antigen that is targeted using the methods and cells described herein is a lineage-specific cell-surface antigen of leukocytes or a subpopulation of leukocytes. In some embodiments, the lineage-specific antigen cell-surface is an antigen that is associated with myeloid cells. In some embodiments, the lineage-specific cell-surface antigen is a cluster of differentiation antigens (CDs). Examples of CD antigens include, without limitation, CD1a, CD1b, CD1c, CD1d, CD1e, CD2, CD3, CD3d, CD3e, CD3g, CD4, CD5, CD6, CD7, CD8a, CD8b, CD9, CD10, CD11a, CD11b, CD11c, CD11d, CDw12, CD13, CD14, CD15, CD16, CD16b, CD17, CD18, CD19, CD20, CD21, CD22, CD23, CD24, CD25, CD26, CD27, CD28, CD29, CD30, CD31, CD32a, CD32b, CD32c, CD33, CD34, CD35, CD36, CD37, CD38, CD39, CD40, CD41, CD42a, CD42b, CD42c, CD42d, CD43, CD44, CD45, CD45RA, CD45RB, CD45RC, CD45RO, CD46, CD47, CD48, CD49a, CD49b, CD49c, CD49d, CD49e, CD49f, CD50, CD51, CD52, CD53, CD54, CD55, CD56, CD57, CD58, CD59, CD60a, CD61, CD62E, CD62L, CD62P, CD63, CD64a, CD65, CD65s, CD66a, CD66b, CD66c, CD66F, CD68, CD69, CD70, CD71, CD72, CD73, CD74, CD75, CD75S, CD77, CD79a, CD79b, CD80, CD81, CD82, CD83, CD84, CD85A, CD85C, CD85D, CD85E, CD85F, CD85G, CD85H, CD85I, CD85J, CD85K, CD86, CD87, CD88, CD89, CD90, CD91, CD92, CD93, CD94, CD95, CD96, CD97, CD98, CD99, CD99R, CD100, CD101, CD102, CD103, CD104, CD105, CD106, CD107a, CD107b, CD108, CD109, CD110, CD111, CD112, CD113, CD114, CD115, CD116, CD117, CD118, CD119, CD120a, CD120b, CD121a, CD121b, CD121a, CD121b, CD122, CD123, CD124, CD125, CD126, CD127, CD129, CD130, CD131, CD132, CD133, CD134, CD135, CD136, CD137, CD138, CD139, CD140a, CD140b, CD141, CD142, CD143, CD144, CDw145, CD146, CD147, CD148, CD150, CD152, CD152, CD153, CD154, CD155, CD156a, CD156b, CD156c, CD157, CD158b1, CD158b2, CD158d, CD158e1/e2, CD158f, CD158g, CD158h, CD158i, CD158j, CD158k, CD159a, CD159c, CD160, CD161, CD163, CD164, CD165, CD166, CD167a, CD168, CD169, CD170, CD171, CD172a, CD172b, CD172g, CD173, CD174, CD175, CD175s, CD176, CD177, CD178, CD179a, CD179b, CD180, CD181, CD182, CD183, CD184, CD185, CD186, CD191, CD192, CD193, CD194, CD195, CD196, CD197, CDw198, CDw199, CD200, CD201, CD202b, CD203c, CD204, CD205, CD206, CD207, CD208, CD209, CD210a, CDw210b, CD212, CD213a1, CD213a2, CD215, CD217, CD218a, CD218b, CD220, CD221, CD222, CD223, CD224, CD225, CD226, CD227, CD228, CD229, CD230, CD231, CD232, CD233, CD234, CD235a, CD235b, CD236, CD236R, CD238, CD239, CD240, CD241, CD242, CD243, CD244, CD245, CD246, CD247, CD248, CD249, CD252, CD253, CD254, CD256, CD257, CD258, CD261, CD262, CD263, CD264, CD265, CD266, CD267, CD268, CD269, CD270, CD271, CD272, CD273, CD274, CD275, CD276, CD277, CD278, CD279, CD280, CD281, CD282, CD283, CD284, CD286, CD288, CD289, CD290, CD292, CDw293, CD294, CD295, CD296, CD297, CD298, CD299, CD300a, CD300c, CD300e, CD301, CD302, CD303, CD304, CD305, 306, CD307a, CD307b, CD307c, D307d, CD307e, CD309, CD312, CD314, CD315, CD316, CD317, CD318, CD319, CD320, CD321, CD322, CD324, CD325, CD326, CD327, CD328, CD329, CD331, CD332, CD333, CD334, CD335, CD336, CD337, CD338, CD339, CD340, CD344, CD349, CD350, CD351, CD352, CD353, CD354, CD355, CD357, CD358, CD359, CD360, CD361, CD362 and CD363. See www.bdbiosciences.com/documents/BD_Reagents_CDMarkerHuman_Poster.pdf.

In some embodiments, the lineage-specific cell-surface antigen is CD19, CD20, CD11, CD123, CD56, CD34, CD14, CD33, CD66b, CD41, CD61, CD62, CD235a, CD146, CD326, LMP2, CD22, CD52, CD10, CD3/TCR, CD79/BCR, and CD26.

In some embodiments, the lineage-specific cell-surface antigen is CD33.

Alternatively or in addition, the lineage-specific cell-surface antigen may be a cancer antigen, for example a lineage-specific cell-surface antigen that is differentially present on cancer cells. In some embodiments, the cancer antigen is an antigen that is specific to a tissue or cell lineage. Examples of lineage-specific cell-surface antigen that are associated with a specific type of cancer include, without limitation, CD20, CD22 (Non-Hodgkin's lymphoma, B-cell lymphoma, chronic lymphocytic leukemia (CLL)), CD52 (B-cell CLL), CD33 (Acute myelogenous leukemia (AML)), CD10 (gp100) (Common (pre-B) acute lymphocytic leukemia and malignant melanoma), CD3/T-cell receptor (TCR) (T-cell lymphoma and leukemia), CD79/B-cell receptor (BCR) (B-cell lymphoma and leukemia), CD26 (epithelial and lymphoid malignancies), human leukocyte antigen (HLA)-DR, HLA-DP, and HLA-DQ (lymphoid malignancies), RCAS1 (gynecological carcinomas, biliary adenocarcinomas and ductal adenocarcinomas of the pancreas) as well as prostate specific membrane antigen.

In some embodiments, the lineage-specific cell-surface antigen CD33 and is associated with AML cells. In some embodiments, the lineage-specific cell-surface antigen EMR2 and is associated with AML cells.

B. Antigen-Binding Fragment

Any antibody or an antigen-binding fragment thereof (e.g., which binds CD33 or at least one additional lineage-specific cell-surface antigen, e.g, EMR2) can be used for constructing the agent that targets a lineage-specific cell-surface antigen as described herein. Such an antibody or antigen-binding fragment can be prepared by a conventional method, for example, using hybridoma technology or recombinant technology.

For example, antibodies specific to a lineage-specific antigen of interest can be made by conventional hybridoma technology. The cell-surface lineage-specific antigen, which may be coupled to a carrier protein such as KLH, can be used to immunize a host animal for generating antibodies binding to that complex. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler and Milstein Nature (1975) 256:495-497 or as modified by Buck et al., In Vitro (1982) 18:377-381. Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the TCR-like monoclonal antibodies described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of binding to a lineage-specific antigen. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the lineage-specific antigen. It will be apparent to one of skill in the art that one or more polynucleotide changes can be made to the antibody and still maintain its binding specificity to the target antigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse™ from Amgen, Inc. (Fremont, Calif.) and HuMAb-Mouse™ and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., Annu. Rev. Immunol. (1994) 12:433-455. Alternatively, the phage display technology (McCafferty et al., Nature (1990) 348:552-553) can be used to produce human antibodies and antibody fragments in vitro, from immunoglobulin variable (V) domain gene repertoires from unimmunized donors.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding monoclonal antibodies specific to a target antigen can be readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., Proc. Nat. Acad. Sci. (1984) 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al., Proc. Natl. Acad. Sci. (1984) 81, 6851; Neuberger et al., Nature (1984) 312, 604; and Takeda et al., Nature (1984) 314:452.

Methods for constructing humanized antibodies are also well known in the art. See, e.g., Queen et al., Proc. Natd. Acad. Sci. (1989) 86:10029-10033. In one example, variable regions of VH and VL of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human VH and VL chains having amino acid sequences that are homologous to those of the parent non-human antibody are identified from any antibody gene database using the parent VH and VL sequences as search queries. Human VH and VL acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a lineage-specific antigen can be identified from the library following routine procedures. Positive clones can be subjected to further screening to identify those that bind lineage-specific cell-surface antigen.

In some instances, lineage-specific cell-surface antigen of interest is CD33 and the antigen-binding fragment specifically binds CD33, for example, human CD33. Amino acid and nucleic acid sequences of an exemplary heavy chain variable region and light chain variable region of an anti-human CD33 antibody are provided below. The CDR sequences are shown in boldface and underlined in the amino acid sequences.

Amino acid sequence of anti-CD33
Heavy Chain Variable Region
(SEQ ID NO: 5)
QVQLQQPGAEVVKPGASVKMSCKASGYTFTSYYIHWIKQT
PGQGLEWVGVIYPGNDDISYNQKFQGKATLTADKSSTTAY
MQLSSLTSEDSAVYYCAREVRLRYFDVWGQGTTVTVSS
Nucleic acid sequence of anti-CD33
Heavy Chain Variable Region
(SEQ ID NO: 6)
CAGGTGCAGCTGCAGCAGCCCGGCGCCGAGGTGGTGAAGC
CCGGCGCCAGCGTGAAGATGAGCTGCAAGGCCAGCGGCTA
CACCTTCACCAGCTACTACATCCACTGGATCAAGCAGACC
CCCGGCCAGGGCCTGGAGTGGGTGGGCGTGATCTACCCCG
GCAACGACGACATCAGCTACAACCAGAAGTTCCAGGGCAA
GGCCACCCTGACCGCCGACAAGAGCAGCACCACCGCCTAC
ATGCAGCTGAGCAGCCTGACCAGCGAGGACAGCGCCGTGT
ACTACTGCGCCAGGGAGGTGAGGCTGAGGTACTTCGACGT
GTGGGGCCAGGGCACCACCGTGACCGTGAGCAGC
Amino acid sequence of anti-CD33
Light Chain Variable Region
(SEQ ID NO: 7)
EIVLTQSPGSLAVSPGERVTMSCKSSQSVFFSSSQKNYLA
WYQQIPGQSPRLLIYWASTRESGVPDRFTGSGSGTDFTLT
ISSVQPEDLAIYYCHQYLSSRTFGQGTKLEIKR
Nucleic acid sequence of anti-CD33
Heavy Chain Variable Region
(SEQ ID NO: 8)
GAGATCGTGCTGACCCAGAGCCCCGGCAGCCTGGCCGTGA
GCCCCGGCGAGAGGGTGACCATGAGCTGCAAGAGCAGCCA
GAGCGTGTTCTTCAGCAGCAGCCAGAAGAACTACCTGGCC
TGGTACCAGCAGATCCCCGGCCAGAGCCCCAGGCTGCTGA
TCTACTGGGCCAGCACCAGGGAGAGCGGCGTGCCCGACAG
GTTCACCGGCAGCGGCAGCGGCACCGACTTCACCCTGACC
ATCAGCAGCGTGCAGCCCGAGGACCTGGCCATCTACTACT
GCCACCAGTACCTGAGCAGCAGGACCTTCGGCCAGGGCAC
CAAGCTGGAGATCAAGAGG

The anti-CD33 antibody binding fragment for use in constructing the agent that targets CD33 as described herein may comprise the same heavy chain and/or light chain CDR regions as those in SEQ ID NO: 5 and SEQ ID NO: 7. Such antibodies may comprise amino acid residue variations in one or more of the framework regions. In some instances, the anti-CD33 antibody fragment may comprise a heavy chain variable region that shares at least 70% sequence identity (e.g., 75%, 80%, 85%, 90%, 95%, or higher) with SEQ ID NO: 5 and/or may comprise a light chain variable region that shares at least 70% sequence identity (e.g., 75%, 80%, 85%, 90%, 95%, or higher) with SEQ ID NO: 7.

The “percent identity” of two amino acid sequences is determined using the algorithm of Karlin and Altschul Proc. Natl. Acad. Sci. USA (1990) 87:2264-68, modified as in Karlin and Altschul Proc. Natl. Acad. Sci. USA (1993) 90:5873-77. Such an algorithm is incorporated into the NBLAST and XBLAST programs (version 2.0) of Altschul, et al. J. Mol. Biol. (1990) 215:403-10. BLAST protein searches can be performed with the XBLAST program, score=50, wordlength=3 to obtain amino acid sequences homologous to the protein molecules of the present disclosure. Where gaps exist between two sequences, Gapped BLAST can be utilized as described in Altschul et al., Nucleic Acids Res. (1997) 25(17):3389-3402. When utilizing BLAST and Gapped BLAST programs, the default parameters of the respective programs (e.g., XBLAST and NBLAST) can be used.

C. Immune Cells Expressing Chimeric Receptors

In some embodiments, the agent that targets a lineage-specific cell-surface antigen as described herein is an immune cell that expresses a chimeric receptor, which comprises an antigen-binding fragment (e.g., a single-chain antibody) capable of binding to the lineage-specific antigen (e.g., CD33, EMR2). Recognition of a target cell (e.g., a cancer cell) having the lineage-specific antigen on its cell surface by the antigen-binding fragment of the chimeric receptor transduces an activation signal to the signaling domain(s) (e.g., co-stimulatory signaling domain and/or the cytoplasmic signaling domain) of the chimeric receptor, which may activate an effector function in the immune cell expressing the chimeric receptor.

As used herein, a chimeric receptor refers to a non-naturally occurring molecule that can be expressed on the surface of a host cell and comprises an antigen-binding fragment that binds to a cell-surface lineage-specific antigen. In general, chimeric receptors comprise at least two domains that are derived from different molecules. In addition to the antigen-binding fragment described herein, the chimeric receptor may further comprise one or more of a hinge domain, a transmembrane domain, at least one co-stimulatory domain, and a cytoplasmic signaling domain. In some embodiments, the chimeric receptor comprises from N terminus to C terminus, an antigen-binding fragment that binds to a cell-surface lineage-specific antigen, a hinge domain, a transmembrane domain, and a cytoplasmic signaling domain. In some embodiments, the chimeric receptor further comprises at least one co-stimulatory domain.

In some embodiments, the chimeric receptors described herein comprise a hinge domain, which may be located between the antigen-binding fragment and a transmembrane domain. A hinge domain is an amino acid segment that is generally found between two domains of a protein and may allow for flexibility of the protein and movement of one or both of the domains relative to one another. Any amino acid sequence that provides such flexibility and movement of the antigen-binding fragment relative to another domain of the chimeric receptor can be used.

The hinge domain may contain about 10-200 amino acids, e.g., 15-150 amino acids, 20-100 amino acids, or 30-60 amino acids. In some embodiments, the hinge domain may be of about 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, or 200 amino acids in length.

In some embodiments, the hinge domain is a hinge domain of a naturally occurring protein. Hinge domains of any protein known in the art to comprise a hinge domain are compatible for use in the chimeric receptors described herein. In some embodiments, the hinge domain is at least a portion of a hinge domain of a naturally occurring protein and confers flexibility to the chimeric receptor. In some embodiments, the hinge domain is of CD8α or CD28 α. In some embodiments, the hinge domain is a portion of the hinge domain of CD8α, e.g., a fragment containing at least 15 (e.g., 20, 25, 30, 35, or 40) consecutive amino acids of the hinge domain of CD8α or CD28a.

Hinge domains of antibodies, such as an IgG, IgA, IgM, IgE, or IgD antibody, are also compatible for use in the chimeric receptors described herein. In some embodiments, the hinge domain is the hinge domain that joins the constant domains CH1 and CH2 of an antibody. In some embodiments, the hinge domain is of an antibody and comprises the hinge domain of the antibody and one or more constant regions of the antibody. In some embodiments, the hinge domain comprises the hinge domain of an antibody and the CH3 constant region of the antibody. In some embodiments, the hinge domain comprises the hinge domain of an antibody and the CH2 and CH3 constant regions of the antibody. In some embodiments, the antibody is an IgG, IgA, IgM, IgE, or IgD antibody. In some embodiments, the antibody is an IgG antibody. In some embodiments, the antibody is an IgG1, IgG2, IgG3, or IgG4 antibody. In some embodiments, the hinge region comprises the hinge region and the CH2 and CH3 constant regions of an IgG1 antibody. In some embodiments, the hinge region comprises the hinge region and the CH3 constant region of an IgG1 antibody.

Also within the scope of the present disclosure are chimeric receptors comprising a hinge domain that is a non-naturally occurring peptide. In some embodiments, the hinge domain between the C-terminus of the extracellular ligand-binding domain of an Fc receptor and the N-terminus of the transmembrane domain is a peptide linker, such as a (GlyxSer)n linker), wherein x and n, independently can be an integer between 3 and 12, including 3, 4, 5, 6, 7, 8, 9, 10, 11, 12, or more.

Additional peptide linkers that may be used in a hinge domain of the chimeric receptors described herein are known in the art. See, e.g., Wriggers et al. Current Trends in Peptide Science (2005) 80(6): 736-746 and PCT Publication WO 2012/088461.

In some embodiments, the chimeric receptors described herein may comprise a transmembrane domain. The transmembrane domain for use in the chimeric receptors can be in any form known in the art. As used herein, a “transmembrane domain” refers to any protein structure that is thermodynamically stable in a cell membrane, preferably a eukaryotic cell membrane. Transmembrane domains compatible for use in the chimeric receptors used herein may be obtained from a naturally occurring protein. Alternatively, the transmembrane domain may be a synthetic, non-naturally occurring protein segment, e.g., a hydrophobic protein segment that is thermodynamically stable in a cell membrane.

Transmembrane domains are classified based on the transmembrane domain topology, including the number of passes that the transmembrane domain makes across the membrane and the orientation of the protein. For example, single-pass membrane proteins cross the cell membrane once, and multi-pass membrane proteins cross the cell membrane at least twice (e.g., 2, 3, 4, 5, 6, 7 or more times). In some embodiments, the transmembrane domain is a single-pass transmembrane domain. In some embodiments, the transmembrane domain is a single-pass transmembrane domain that orients the N terminus of the chimeric receptor to the extracellular side of the cell and the C terminus of the chimeric receptor to the intracellular side of the cell. In some embodiments, the transmembrane domain is obtained from a single pass transmembrane protein. In some embodiments, the transmembrane domain is of CD8α. In some embodiments, the transmembrane domain is of CD28. In some embodiments, the transmembrane domain is of ICOS.

In some embodiments, the chimeric receptors described herein comprise one or more costimulatory signaling domains. The term “co-stimulatory signaling domain,” as used herein, refers to at least a portion of a protein that mediates signal transduction within a cell to induce an immune response, such as an effector function. The co-stimulatory signaling domain of the chimeric receptor described herein can be a cytoplasmic signaling domain from a co-stimulatory protein, which transduces a signal and modulates responses mediated by immune cells, such as T cells, NK cells, macrophages, neutrophils, or eosinophils.

In some embodiments, the chimeric receptor comprises more than one (at least 2, 3, 4, or more) co-stimulatory signaling domains. In some embodiments, the chimeric receptor comprises more than one co-stimulatory signaling domains obtained from different costimulatory proteins. In some embodiments, the chimeric receptor does not comprise a co-stimulatory signaling domain.

In general, many immune cells require co-stimulation, in addition to stimulation of an antigen-specific signal, to promote cell proliferation, differentiation and survival, and to activate effector functions of the cell. Activation of a co-stimulatory signaling domain in a host cell (e.g., an immune cell) may induce the cell to increase or decrease the production and secretion of cytokines, phagocytic properties, proliferation, differentiation, survival, and/or cytotoxicity. The co-stimulatory signaling domain of any co-stimulatory protein may be compatible for use in the chimeric receptors described herein. The type(s) of co-stimulatory signaling domain is selected based on factors such as the type of the immune cells in which the chimeric receptors would be expressed (e.g., primary T cells, T cell lines, NK cell lines) and the desired immune effector function (e.g., cytotoxicity). Examples of co-stimulatory signaling domains for use in the chimeric receptors can be the cytoplasmic signaling domain of co-stimulatory proteins, including, without limitation, CD27, CD28, 4-1BB, OX40, CD30, Cd40, PD-1, ICOS, lymphocyte function-associated antigen-1 (LFA-1), CD2, CD7, LIGHT, NKG2C, B7-H3. In some embodiments, the co-stimulatory domain is derived from 4-1BB, CD28, or ICOS. In some embodiments, the costimulatory domain is derived from CD28 and chimeric receptor comprises a second co-stimulatory domain from 4-1BB or ICOS.

In some embodiments, the costimulatory domain is a fusion domain comprising more than one costimulatory domain or portions of more than one costimulatory domain. In some embodiments, the costimulatory domain is a fusion of costimulatory domains from CD28 and ICOS.

In some embodiments, the chimeric receptors described herein comprise a cytoplasmic signaling domain. Any cytoplasmic signaling domain can be used in the chimeric receptors described herein. In general, a cytoplasmic signaling domain relays a signal, such as interaction of an extracellular ligand-binding domain with its ligand, to stimulate a cellular response, such as inducing an effector function of the cell (e.g., cytotoxicity).

As will be evident to one of ordinary skill in the art, a factor involved in T cell activation is the phosphorylation of immunoreceptor tyrosine-based activation motif (ITAM) of a cytoplasmic signaling domain. Any ITAM-containing domain known in the art may be used to construct the chimeric receptors described herein. In general, an ITAM motif may comprise two repeats of the amino acid sequence YxxL/I separated by 6-8 amino acids, wherein each x is independently any amino acid, producing the conserved motif YxxL/Ix(6-8)YxxL/I. In some embodiments, the cytoplasmic signaling domain is from CD3ζ.

Exemplary chimeric receptors are provided in Tables 2 and 3 below.

TABLE 2
Exemplary components of a chimeric receptor

| Chimeric receptor component | Amino acid sequence |
|---|---|
| Antigen-binding fragment | Light chain- GSTSSGSGKPGSGEGSTKG |
| | (SEQ ID NO: 9)-Heavy chain |
| CD28 costimulatory domain | IEVMYPPPYLDNEKSNGTIIHVKGKHLCPSP |
| | LFPGPSKPFWVLVVVGGVLACYSLLVTVA |
| | FIIFWVRSKRSRLLHSDYMNMTPRRPGPTR |
| | KHYQPYAPPRDFAAYRS (SEQ ID NO: 10) |
| ICOS costimulatory domain (boldface), | LSIFDPPPFKVTLTGGYLHIYESQLCCQLKF |
| ICOS transmembrane domain (italics) | WLPIGCAAFVVVCILGCILICWLTKKKYSSS |
| and a portion of the extracellular | VHDPNGEYMFMRAVNTAKKSRLTDVTL |
| domain of ICOS (underlined) | (SEQ ID NO: 11) |
| ICOS costimulatory domain | CWLTKKKYSSSVHDPNGEYMFMRAVNTA |
| | KKSRLTDVTL (SEQ ID NO: 12) |
| CD28/ICOS chimera | IEVMYPPPYLDNEKSNGTIIHVKGKHLCPS |
| (the ICOS portion | PLFPGPSKPFWVLVVVGGVLACYSLLVTVA |
| shown in underline) | FIIFWVRSKRSRLLHSDYMFMRAVNTAKK |
| including the hinge | SRLTDVTL (SEQ ID NO: 13) |
| domain (italics) | |
| and transmembrane | |
| domain (bold) from CD28 | |
| CD3ζ cytoplasmic | RVKFSRSADAPAYQQGQNQLYNELNLGRR |
| signaling domain | EEYDVLDKRRGRDPEMGGKPQRRKNPQE |
| | GLYNELQKDKMAEAYSEIGMKGERRRGK |
| | GHDGLYQGLSTATKDTYDALHMQALPPR |
| | (SEQ ID NO: 14) |

The nucleic acid sequence of exemplary components for construction of a chimeric receptor are provided below.

CD28 intracellular signaling domain-
DNA-Human
(SEQ ID NO: 15)
ATTGAAGTTATGTATCCTCCTCCTTACCTAGACAATGAGA
AGAGCAATGGAACCATTATCCATGTGAAAGGGAAACACCT
TTGTCCAAGTCCCCTATTTCCCGGACCTTCTAAGCCCTTT
TGGGTGCTGGTGGTGGTTGGTGGAGTCCTGGCTTGCTATA
GCTTGCTAGTAACAGTGGCCTTTATTATTTTCTGGGTGAG
GAGTAAGAGGAGCAGGCTCCTGCACAGTGACTACATGAAC
ATGACTCCCCGCCGCCCCGGGCCCACCCGCAAGCATTACC
AGCCCTATGCCCCACCACGCGACTTCGCAGCCTATCGCTC
CAGAGTGAAGTTCAGCAGGAGCGCAGACGCCCCCGCGTAC
CAGCAGGGCCAGAACCAGCTCTATAACGAGCTCAATCTAG
GACGAAGAGAGGAGTACGATGTTTTGGACAAGAGACGTGG
CCGGGACCCTGAGATGGGGGGAAAGCCGAGAAGGAAGAAC
CCTCAGGAAGGCCTGTACAATGAACTGCAGAAAGATAAGA
TGGCGGAGGCCTACAGTGAGATTGGGATGAAAGGCGAGCG
CCGGAGGGGCAAGGGGCACGATGGCCTTTACCAGGGTCTC
AGTACAGCCACCAAGGACACCTACGACGCCCTTCACATGC
AGGCCCTGCCCCCTCGC
ICOS intracellular signaling domain-
DNA-Human
(SEQ ID NO: 16)
CTATCAATTTTTGATCCTCCTCCTTTTAAAGTAACTCTTA
CAGGAGGATATTTGCATATTTATGAATCACAACTTTGTTG
CCAGCTGAAGTTCTGGTTACCCATAGGATGTGCAGCCTTT
GTTGTAGTCTGCATTTTGGGATGCATACTTATTTGTTGGC
TTACAAAAAAGAAGTATTCATCCAGTGTGCACGACCCTAA
CGGTGAATACATGTTCATGAGAGCAGTGAACACAGCCAAA
AAATCTAGACTCACAGATGTGACCCTAAGAGTGAAGTTCA
GCAGGAGCGCAGACGCCCCCGCGTACCAGCAGGGCCAGAA
CCAGCTCTATAACGAGCTCAATCTAGGACGAAGAGAGGAG
TACGATGTTTTGGACAAGAGACGTGGCCGGGACCCTGAGA
TGGGGGGAAAGCCGAGAAGGAAGAACCCTCAGGAAGGCCT
GTACAATGAACTGCAGAAAGATAAGATGGCGGAGGCCTAC
AGTGAGATTGGGATGAAAGGCGAGCGCCGGAGGGGCAAGG
GGCACGATGGCCTTTACCAGGGTCTCAGTACAGCCACCAA
GGACACCTACGACGCCCTTCACATGCAGGCCCTGCCCCCT
CGC
CD28/ICOS COSTIMULATORY SIGNALING
REGION-DNA-Human
(SEQ ID NO: 17)
ATTGAAGTTATGTATCCTCCTCCTTACCTAGACAATGAGA
AGAGCAATGGAACCATTATCCATGTGAAAGGGAAACACCT
TTGTCCAAGTCCCCTATTTCCCGGACCTTCTAAGCCCTTT
TGGGTGCTGGTGGTGGTTGGTGGAGTCCTGGCTTGCTATA
GCTTGCTAGTAACAGTGGCCTTTATTATTTTCTGGGTGAG
GAGTAAGAGGAGCAGGCTCCTGCACAGTGACTACATGTTC
ATGAGAGCAGTGAACACAGCCAAAAAATCTAGACTCACAG
ATGTGACCCTAAGAGTGAAGTTCAGCAGGAGCGCAGACGC
CCCCGCGTACCAGCAGGGCCAGAACCAGCTCTATAACGAG
CTCAATCTAGGACGAAGAGAGGAGTACGATGTTTTGGACA
AGAGACGTGGCCGGGACCCTGAGATGGGGGGAAAGCCGAG
AAGGAAGAACCCTCAGGAAGGCCTGTACAATGAACTGCAG
AAAGATAAGATGGCGGAGGCCTACAGTGAGATTGGGATGA
AAGGCGAGCGCCGGAGGGGCAAGGGGCACGATGGCCTTTA
CCAGGGTCTCAGTACAGCCACCAAGGACACCTACGACGCC
CTTCACATGCAGGCCCTGCCCCCTCGC

In some embodiments, the nucleic acid sequence encodes an antigen binding fragment that binds to CD33 and comprises a heavy chain variable region which has the same CDRs as the CDRs in SEQ ID NO: 5 and a light chain variable region which has the same CDRs as the CDRs in SEQ ID NO: 7. In some embodiments, the antigen-binding fragment comprises a heavy chain variable region as provided by SEQ ID NO: 5 and a light chain variable region as provided by SEQ ID NO: 7. In some embodiments, the chimeric receptor further comprises at least a transmembrane domain and a cytoplasmic signaling domain. In some embodiments, the chimeric receptor further comprises a hinge domain and/or a co-stimulatory signaling domain.

Table 3 provides exemplary chimeric receptors described herein. The exemplary constructs have from N-terminus to C-terminus, the antigen-binding fragment, the transmembrane domain, and a cytoplasmic signaling domain. In some examples, the chimeric receptor further comprises a hinge domain located between the antigen-binding fragment and the transmembrane domain. In some examples, the chimeric receptor further comprises one or more co-stimulatory domains, which may be located between the transmembrane domain and the cytoplasmic signaling domain.

TABLE 3
Exemplary chimeric receptors

| | | Antigen- | | | | | |
|---|---|---|---|---|---|---|---|
| | | binding | | | | | |
| | | fragment | Hinge | Transmembrane | Signaling | Signaling | Signaling |
| Constructs | Vector | specificity | domain | domain | domain 1 | domain 2 | domain 3 |
| CART1 | HIVzs- | CD33 | CD8α | CD8 | 4-1BB | CD3 ζ | None |
| (SEQ ID | Gfp | | | | | | |
| NO: 18) | | | | | | | |
| CART2 | HIVzs- | CD33 | CD8α | CD28 | CD28 | CD3 ζ | None |
| (SEQ ID | Gfp | | | | | | |
| NO: 19) | | | | | | | |
| CART3 | HIVzs- | CD33 | CD8α | CD28 | CD28 | 4-1BB | CD3 ζ |
| (SEQ ID | Gfp | | | | | | |
| NO: 20) | | | | | | | |
| CART8 | HIVzs- | CD33 | CD8α | ICOS | ICOS | 4-1BB | CD3 ζ |
| (SEQ ID | Gfp | | | | | | |
| NO: 21) | | | | | | | |
| CART4dual | HIVzs- | CD19 | CD8α | CD28 | CD3 ζ | — | — |
| (SEQ ID | dT | | | | | | |
| NO: 22) | | | | | | | |
| CART5dual | HIVzs- | CD33 | CD8α | CD28 | CD28 | — | — |
| (SEQ ID | Gfp | | | | | | |
| NO: 23) | | | | | | | |
| CART6 | HIVzs- | CD19 | CD8α | CD28 | CD28 | CD3 ζ | — |
| (SEQ ID | dT | | | | | | |
| NO: 24) | | | | | | | |
| CART7 | HIVzs- | CD33 | CD28 | CD28 | CD28 | CD3 ζ | — |
| (SEQ ID | Gfp | | hge | | | | |
| NO: 25) | | | | | | | |

Amino acid sequences of the example chimeric receptors listed in Table 3 above are provided below:

CART1 amino acid sequence
(SEQ ID NO: 18)
MWLQSLLLLGTVACSISEIVLTQSPGSLAVSPGERVTMSC
KSSQSVFFSSSQKNYLAWYQQIPGQSPRLLIYWASTRESG
VPDRFTGSGSGTDFTLTISSVQPEDLAIYYCHQYLSSRTF
GQGTKLEIKRGSTSGSGKPGSGEGSTKGQVQLQQPGAEVV
KPGASVKMSCKASGYTFTSYYIHWIKQTPGQGLEWVGVIY
PGNDDISYNQKFQGKATLTADKSSTTAYMQLSSLTSEDSA
VYYCAREVRLRYFDVWGQGTTVTVSSALSNSIMYFSHFVP
VFLPAKPTTTPAPRPPTPAPTIASQPLSLRPEASRPAAGG
AVHTRGLDIYIWAPLAGTCGVLLLSLVITKRGRKKLLYIF
KQPFMRPVQTTQEEDGCSCRFPEEEEGGCELRVKFSRSAD
APAYQQGQNQLYNELNLGRREEYDVLDKRRGRDPEMGGKP
RRKNPQEGLYNELQKDKMAEAYSEIGMKGERRRGKGHDGL
YQGLSTATKDTYDALHMQALPPR
CART2 amino acid sequence
(SEQ ID NO: 19)
MWLQSLLLLGTVACSISEIVLTQSPGSLAVSPGERVTMSC
KSSQSVFFSSSQKNYLAWYQQIPGQSPRLLIYWASTRESG
VPDRFTGSGSGTDFTLTISSVQPEDLAIYYCHQYLSSRTF
GQGTKLEIKRGSTSGSGKPGSGEGSTKGQVQLQQPGAEVV
KPGASVKMSCKASGYTFTSYYIHWIKQTPGQGLEWVGVIY
PGNDDISYNQKFQGKATLTADKSSTTAYMQLSSLTSEDSA
VYYCAREVRLRYFDVWGQGTTVTVSALSNSIMYFSHFVPV
FLPAKPTTTPAPRPPTPAPTIASQPLSLRPEASRPAAGGA
VHTRGLDKPFWVLVVVGGVLACYSLLVTVAFIIFWVRSKR
SRLLHSDYMNMTPRRPGPTRKHYQPYAPPRDFAAYRSRVK
FSRSADAPAYQQGQNQLYNELNLGRREEYDVLDKRRGRDP
EMGGKPRRKNPQEGLYNELQKDKMAEAYSEIGMKGERRRG
KGHDGLYQGLSTATKDTYDALHMQALPPR
CART3 amino acid sequence
(SEQ ID NO: 20)
MWLQSLLLLGTVACSISEIVLTQSPGSLAVSPGERVTMSC
KSSQSVFFSSSQKNYLAWYQQIPGQSPRLLIYWASTRESG
VPDRFTGSGSGTDFTLTISSVQPEDLAIYYCHQYLSSRTF
GQGTKLEIKRGSTSGSGKPGSGEGSTKGQVQLQQPGAEVV
KPGASVKMSCKASGYTFTSYYIHWIKQTPGQGLEWVGVIY
PGNDDISYNQKFQGKATLTADKSSTTAYMQLSSLTSEDSA
VYYCAREVRLRYFDVWGQGTTVTVSSALSNSIMYFSHFVP
VFLPAKPTTTPAPRPPTPAPTIASQPLSLRPEASRPAAGG
AVHTRGLDKPFWVLVVVGGVLACYSLLVTVAFIIFWVRSK
RSRLLHSDYMNMTPRRPGPTRKHYQPYAPPRDFAAYRSKR
GRKKLLYIFKQPFMRPVQTTQEEDGCSCRFPEEEEGGCEL
RVKFSRSADAPAYQQGQNQLYNELNLGRREEYDVLDKRRG
RDPEMGGKPRRKNPQEGLYNELQKDKMAEAYSEIGMKGER
RRGKGHDGLYQGLSTATKDTYDALHMQALPPR
CART8 amino acid sequence
(SEQ ID NO: 21)
MWLQSLLLLGTVACSISEIVLTQSPGSLAVSPGERVTMSC
KSSQSVFFSSSQKNYLAWYQQIPGQSPRLLIYWASTRESG
VPDRFTGSGSGTDFTLTISSVQPEDLAIYYCHQYLSSRTF
GQGTKLEIKRGSTSGSGKPGSGEGSTKGQVQLQQPGAEVV
KPGASVKMSCKASGYTFTSYYIHWIKQTPGQGLEWVGVIY
PGNDDISYNQKFQGKATLTADKSSTTAYMQLSSLTSEDSA
VYYCAREVRLRYFDVWGQGTTVTVSSALSNSIMYFSHFVP
VFLPAKPTTTPAPRPPTPAPTIASQPLSLRPEASRPAAGG
AVHTRGLDFWLPIGCAAFVVVCILGCILICWLTKKKYSSS
VHDPNGEYMFMRAVNTAKKSRLTDVTLTKRGRKKLLYIFK
QPFMRPVQTTQEEDGCSCRFPEEEEGGCELRVKFSRSADA
PAYQQGQNQLYNELNLGRREEYDVLDKRRGRDPEMGGKPR
RKNPQEGLYNELQKDKMAEAYSEIGMKGERRRGKGHDGLY
QGLSTATKDTYDALHMQALPPR
CART4dual amino acid sequence
(SEQ ID NO: 22)
MWLQSLLLLGTVACSISIQMTQTTSSLSASLGDRVTISCR
ASQDISKYLNWYQQKPDGTVKLLIYHTSRLHSGVPSRFSG
SGSGTDYSLTISNLEQEDIATYFCQQGNTLPYTFGGGTKL
EIGSTSGSGKPGSGEGSTKGLQESGPGLVAPSQSLSVTCT
VSGVSLPDYGVSWIRQPPRKGLEWLGVIWGSETTYYNSAL
KSRLTIIKDNSKSQVFLKMNSLQTDDTAIYYCAKHYYYGG
SYAMDYWGQGTSVTVSALSNSIMYFSHFVPVFLPAKPTTT
PAPRPPTPAPTIASQPLSLRPEASRPAAGGAVHTRGLDKP
FWVLVVVGGVLACYSLLVTVAFIIFWVRVKFSRSADAPAY
QQGQNQLYNELNLGRREEYDVLDKRRGRDPEMGGKPRRKN
PQEGLYNELQKDKMAEAYSEIGMKGERRRGKGHDGLYQGL
STATKDTYDALHMQALPPR
CART5dual amino acid sequence
(SEQ ID NO: 23)
MWLQSLLLLGTVACSISEIVLTQSPGSLAVSPGERVTMSC
KSSQSVFFSSSQKNYLAWYQQIPGQSPRLLIYWASTRESG
VPDRFTGSGSGTDFTLTISSVQPEDLAIYYCHQYLSSRTF
GQGTKLEIKRGSTSGSGKPGSGEGSTKGQVQLQQPGAEVV
KPGASVKMSCKASGYTFTSYYIHWIKQTPGQGLEWVGVIY
PGNDDISYNQKFQGKATLTADKSSTTAYMQLSSLTSEDSA
VYYCAREVRLRYFDVWGQGTTVTVSSALSNSIMYFSHFVP
VFLPAKPTTTPAPRPPTPAPTIASQPLSLRPEASRPAAGG
AVHTRGLDKPFWVLVVVGGVLACYSLLVTVAFIIFWVRSK
RSRLLHSDYMNMTPRRPGPTRKHYQPYAPPRDFAAYRS
CART6 amino acid sequence
(SEQ ID NO: 24)
MWLQSLLLLGTVACSISIQMTQTTSSLSASLGDRVTISCR
ASQDISKYLNWYQQKPDGTVKLLIYHTSRLHSGVPSRFSG
SGSGTDYSLTISNLEQEDIATYFCQQGNTLPYTFGGGTKL
EIGSTSGSGKPGSGEGSTKGLQESGPGLVAPSQSLSVTCT
VSGVSLPDYGVSWIRQPPRKGLEWLGVIWGSETTYYNSAL
KSRLTIIKDNSKSQVFLKMNSLQTDDTAIYYCAKHYYYGG
SYAMDYWGQGTSVTVSALSNSIMYFSHFVPVFLPAKPTTT
PAPRPPTPAPTIASQPLSLRPEASRPAAGGAVHTRGLDKP
FWVLVVVGGVLACYSLLVTVAFIIFWVRSKRSRLLHSDYM
NMTPRRPGPTRKHYQPYAPPRDFAAYRSRVKFSRSADAPA
YQQGQNQLYNELNLGRREEYDVLDKRRGRDPEMGGKPRRK
NPQEGLYNELQKDKMAEAYSEIGMKGERRRGKGHDGLYQG
LSTATKDTYDALHMQALPPR
CART7 amino acid sequence
(SEQ ID NO: 25)
MWLQSLLLLGTVACSISEIVLTQSPGSLAVSPGERVTMSC
KSSQSVFFSSSQKNYLAWYQQIPGQSPRLLIYWASTRESG
VPDRFTGSGSGTDFTLTISSVQPEDLAIYYCHQYLSSRTF
GQGTKLEIKRGSTSGSGKPGSGEGSTKGQVQLQQPGAEVV
KPGASVKMSCKASGYTFTSYYIHWIKQTPGQGLEWVGVIY
PGNDDISYNQKFQGKATLTADKSSTTAYMQLSSLTSEDSA
VYYCAREVRLRYFDVWGQGTTVTVSSIEVMYPPPYLDNEK
SNGTIIHVKGKHLCPSPLFPGPSKPFWVLVVVGGVLACYS
LLVTVAFIIFWVRSKRSRLLHSDYMNMTPRRPGPTRKHYQ
PYAPPRDFAAYRSRVKFSRSADAPAYQQGQNQLYNELNLG
RREEYDVLDKRRGRDPEMGGKPRRKNPQEGLYNELQKDKM
AEAYSEIGMGERRRGKGHDGLYQGLSTATKDTYDALHMQA
LPPR

Nucleic acid sequences of the example chimeric receptors listed in Table 3 above are provided below:

CART1 nucleic acid sequence
(SEQ ID NO: 26)
GGTGTCGTGAGCGGCCGCTGAACTGGCCACCATGTGGCTG
CAGTCTCTGCTGCTGCTGGGCACCGTGGCCTGTAGCATCA
GCGAGATCGTGCTGACCCAGAGCCCTGGCTCTCTGGCTGT
GTCTCCTGGCGAGCGCGTGACCATGAGCTGCAAGAGCAGC
CAGAGCGTGTTCTTCAGCAGCTCCCAGAAGAACTACCTGG
CCTGGTATCAGCAGATCCCCGGCCAGAGCCCCAGACTGCT
GATCTACTGGGCCAGCACCAGAGAAAGCGGCGTGCCCGAT
AGATTCACCGGCAGCGGCTCTGGCACCGACTTCACCCTGA
CAATCAGCAGCGTGCAGCCCGAGGACCTGGCCATCTACTA
CTGCCACCAGTACCTGAGCAGCCGGACCTTTGGCCAGGGC
ACCAAGCTGGAAATCAAGCGGGGCAGCACAAGCGGCAGCG
GAAAGCCTGGATCTGGCGAGGGCTCTACCAAGGGCCAGGT
GCAGCTGCAGCAGCCTGGCGCCGAAGTCGTGAAACCTGGC
GCCTCCGTGAAGATGTCCTGCAAGGCCAGCGGCTACACCT
TCACCAGCTACTACATCCACTGGATCAAGCAGACCCCTGG
ACAGGGCCTGGAATGGGTGGGAGTGATCTACCCCGGCAAC
GACGACATCAGCTACAACCAGAAGTTCCAGGGCAAGGCCA
CCCTGACCGCCGACAAGTCTAGCACCACCGCCTACATGCA
GCTGTCCAGCCTGACCAGCGAGGACAGCGCCGTGTACTAC
TGCGCCAGAGAAGTGCGGCTGCGGTACTTCGATGTGTGGG
GCCAGGGAACCACCGTGACCGTGTCTAGCGCCCTGAGCAA
CAGCATCATGTACTTCAGCCACTTCGTGCCCGTGTTTCTG
CCCGCCAAGCCTACCACAACCCCTGCCCCTAGACCTCCTA
CCCCAGCCCCTACAATCGCCAGCCAGCCTCTGTCTCTGAG
GCCCGAGGCTTCTAGACCAGCTGCTGGCGGAGCCGTGCAC
ACCAGAGGCCTGGATATCTACATCTGGGCCCCACTGGCCG
GCACCTGTGGCGTGCTGCTGCTGTCTCTCGTGATCACCAA
GAGAGGCCGGAAGAAGCTGCTGTACATCTTCAAGCAGCCC
TTCATGCGGCCCGTGCAGACCACCCAGGAAGAGGACGGCT
GTAGCTGCCGGTTCCCCGAGGAAGAAGAAGGGGGCTGCGA
GCTGAGAGTGAAGTTCAGCAGAAGCGCCGACGCCCCTGCC
TATCAGCAGGGCCAGAACCAGCTGTACAACGAGCTGAACC
TGGGCAGACGGGAAGAGTACGACGTGCTGGACAAGCGGAG
AGGCAGGGACCCTGAGATGGGCGGCAAGCCCAGACGGAAG
AACCCTCAGGAAGGCCTGTATAACGAACTGCAGAAAGACA
AGATGGCCGAGGCCTACTCCGAGATCGGAATGAAGGGCGA
GCGGAGAAGAGGCAAGGGCCACGATGGACTGTACCAGGGC
CTGAGCACCGCCACCAAGGACACCTATGACGCCCTGCACA
TGCAGGCCCTGCCCCCCAGATGAAATTCATCGACGTTAAC
TATTCTAG
CART2 nucleic acid sequence
(SEQ ID NO: 27)
GGTGTCGTGAGCGGCCGCTGAACTGGCCACCATGTGGCTG
CAGTCTCTGCTGCTGCTGGGCACCGTGGCCTGTAGCATCA
GCGAGATCGTGCTGACCCAGAGCCCTGGCTCTCTGGCTGT
GTCTCCTGGCGAGCGCGTGACCATGAGCTGCAAGAGCAGC
CAGAGCGTGTTCTTCAGCAGCTCCCAGAAGAACTACCTGG
CCTGGTATCAGCAGATCCCCGGCCAGAGCCCCAGACTGCT
GATCTACTGGGCCAGCACCAGAGAAAGCGGCGTGCCCGAT
AGATTCACCGGCAGCGGCTCTGGCACCGACTTCACCCTGA
CAATCAGCAGCGTGCAGCCCGAGGACCTGGCCATCTACTA
CTGCCACCAGTACCTGAGCAGCCGGACCTTTGGCCAGGGC
ACCAAGCTGGAAATCAAGCGGGGCAGCACAAGCGGCAGCG
GAAAGCCTGGATCTGGCGAGGGCTCTACCAAGGGCCAGGT
GCAGCTGCAGCAGCCTGGCGCCGAAGTCGTGAAACCTGGC
GCCTCCGTGAAGATGTCCTGCAAGGCCAGCGGCTACACCT
TCACCAGCTACTACATCCACTGGATCAAGCAGACCCCTGG
ACAGGGCCTGGAATGGGTGGGAGTGATCTACCCCGGCAAC
GACGACATCAGCTACAACCAGAAGTTCCAGGGCAAGGCCA
CCCTGACCGCCGACAAGTCTAGCACCACCGCCTACATGCA
GCTGTCCAGCCTGACCAGCGAGGACAGCGCCGTGTACTAC
TGCGCCAGAGAAGTGCGGCTGCGGTACTTCGATGTGTGGG
GCCAGGGAACCACCGTGACCGTGTCTGCCCTGAGCAACAG
CATCATGTACTTCAGCCACTTCGTGCCCGTGTTTCTGCCC
GCCAAGCCTACCACAACCCCTGCCCCTAGACCTCCTACCC
CAGCCCCTACAATCGCCAGCCAGCCTCTGTCTCTGAGGCC
CGAGGCTTCTAGACCAGCTGCTGGCGGAGCCGTGCACACC
AGAGGACTGGACAAGCCCTTCTGGGTGCTGGTGGTCGTGG
GCGGAGTGCTGGCCTGTTACAGCCTGCTCGTGACAGTGGC
CTTCATCATCTTTTGGGTGCGCAGCAAGCGGTCTAGACTG
CTGCACAGCGACTACATGAACATGACCCCCAGAAGGCCAG
GCCCCACCCGGAAGCACTATCAGCCTTACGCCCCTCCCAG
AGACTTCGCCGCCTACCGGTCCAGAGTGAAGTTCAGCAGA
AGCGCCGACGCCCCTGCCTATCAGCAGGGCCAGAACCAGC
TGTACAACGAGCTGAACCTGGGCAGACGGGAAGAGTACGA
CGTGCTGGACAAGAGAAGAGGCCGGGACCCTGAGATGGGC
GGCAAGCCCAGACGGAAGAACCCTCAGGAAGGCCTGTATA
ACGAACTGCAGAAAGACAAGATGGCCGAGGCCTACTCCGA
GATCGGCATGAAGGGCGAACGGCGGAGAGGCAAGGGACAC
GATGGACTGTACCAGGGCCTGAGCACCGCCACCAAGGACA
CCTATGACGCCCTGCACATGCAGGCCCTGCCCCCCAGATG
AAATTCATCGACGTTAACTATTCTAG
CART3 nucleic acid sequence
(SEQ ID NO: 28)
GGTGTCGTGAGCGGCCGCTGAACTGGCCACCATGTGGCTG
CAGTCTCTGCTGCTGCTGGGCACCGTGGCCTGTAGCATCA
GCGAGATCGTGCTGACCCAGAGCCCTGGCTCTCTGGCTGT
GTCTCCTGGCGAGCGCGTGACCATGAGCTGCAAGAGCAGC
CAGAGCGTGTTCTTCAGCAGCTCCCAGAAGAACTACCTGG
CCTGGTATCAGCAGATCCCCGGCCAGAGCCCCAGACTGCT
GATCTACTGGGCCAGCACCAGAGAAAGCGGCGTGCCCGAT
AGATTCACCGGCAGCGGCTCTGGCACCGACTTCACCCTGA
CAATCAGCAGCGTGCAGCCCGAGGACCTGGCCATCTACTA
CTGCCACCAGTACCTGAGCAGCCGGACCTTTGGCCAGGGC
ACCAAGCTGGAAATCAAGCGGGGCAGCACAAGCGGCAGCG
GAAAGCCTGGATCTGGCGAGGGCTCTACCAAGGGCCAGGT
GCAGCTGCAGCAGCCTGGCGCCGAAGTCGTGAAACCTGGC
GCCTCCGTGAAGATGTCCTGCAAGGCCAGCGGCTACACCT
TCACCAGCTACTACATCCACTGGATCAAGCAGACCCCTGG
ACAGGGCCTGGAATGGGTGGGAGTGATCTACCCCGGCAAC
GACGACATCAGCTACAACCAGAAGTTCCAGGGCAAGGCCA
CCCTGACCGCCGACAAGTCTAGCACCACCGCCTACATGCA
GCTGTCCAGCCTGACCAGCGAGGACAGCGCCGTGTACTAC
TGCGCCAGAGAAGTGCGGCTGCGGTACTTCGATGTGTGGG
GCCAGGGAACCACCGTGACCGTGTCTAGCGCCCTGAGCAA
CAGCATCATGTACTTCAGCCACTTCGTGCCCGTGTTTCTG
CCCGCCAAGCCTACCACAACCCCTGCCCCTAGACCTCCTA
CCCCAGCCCCTACAATCGCCAGCCAGCCTCTGTCTCTGAG
GCCCGAGGCTTCTAGACCAGCTGCTGGCGGAGCCGTGCAC
ACCAGAGGACTGGACAAGCCCTTCTGGGTGCTGGTGGTCG
TGGGCGGAGTGCTGGCCTGTTACAGCCTGCTCGTGACAGT
GGCCTTCATCATCTTTTGGGTGCGCAGCAAGCGGTCTAGA
CTGCTGCACAGCGACTACATGAACATGACCCCCAGAAGGC
CAGGCCCCACCCGGAAGCACTATCAGCCTTACGCCCCTCC
CAGAGACTTCGCCGCCTACAGATCCAAGAGAGGCCGGAAG
AAGCTGCTGTACATCTTCAAGCAGCCCTTCATGCGGCCCG
TGCAGACCACCCAGGAAGAGGACGGCTGTAGCTGCCGGTT
CCCCGAGGAAGAAGAAGGGGGCTGCGAGCTGAGAGTGAAG
TTCAGCAGAAGCGCCGACGCCCCTGCCTATCAGCAGGGCC
AGAACCAGCTGTACAACGAGCTGAACCTGGGCAGACGGGA
AGAGTACGACGTGCTGGACAAGAGAAGAGGCCGGGACCCT
GAGATGGGCGGCAAGCCCAGACGGAAGAACCCTCAGGAAG
GCCTGTATAACGAACTGCAGAAAGACAAGATGGCCGAGGC
CTACTCCGAGATCGGAATGAAGGGCGAGCGGCGGAGAGGC
AAGGGACACGATGGACTGTACCAGGGCCTGAGCACCGCCA
CCAAGGACACCTATGACGCCCTGCACATGCAGGCCCTGCC
CCCCAGATGAAATTCATCGACGTTAACTATTCTAG
CART4dual nucleic acid sequence
(SEQ ID NO: 29)
GGTGTCGTGAGCGGCCGCTGAACTGGCCACCATGTGGCTG
CAGTCTCTGCTGCTGCTGGGCACCGTGGCCTGCAGCATCA
GCATCCAGATGACCCAGACCACCAGCAGCCTGAGCGCCAG
CCTGGGCGATAGAGTGACCATCAGCTGCAGAGCCAGCCAG
GACATCAGCAAGTACCTGAACTGGTATCAGCAGAAACCCG
ACGGCACCGTGAAGCTGCTGATCTACCACACCAGCAGACT
GCACAGCGGCGTGCCCTCTAGATTTTCCGGCAGCGGCTCC
GGCACCGACTACAGCCTGACCATCTCCAACCTGGAACAGG
AAGATATCGCTACCTACTTCTGTCAGCAAGGCAACACCCT
GCCCTACACCTTCGGCGGAGGCACCAAGCTGGAAATCGGC
AGCACAAGCGGCTCTGGCAAGCCTGGATCTGGCGAGGGCT
CTACCAAGGGCCTGCAGGAATCTGGCCCTGGACTGGTGGC
CCCTAGCCAGAGCCTGTCTGTGACCTGTACCGTGTCCGGC
GTGTCCCTGCCTGACTATGGCGTGTCCTGGATCAGACAGC
CCCCCAGAAAGGGCCTGGAATGGCTGGGAGTGATCTGGGG
CAGCGAGACAACCTACTACAACAGCGCCCTGAAGTCCCGG
CTGACCATCATCAAGGACAACTCCAAGAGCCAGGTGTTCC
TGAAGATGAACAGCCTGCAGACCGACGACACCGCCATCTA
CTACTGCGCCAAGCACTACTACTACGGCGGCAGCTACGCC
ATGGACTACTGGGGCCAGGGCACAAGCGTGACCGTGTCTG
CCCTGAGCAACAGCATCATGTACTTCAGCCACTTCGTGCC
CGTGTTTCTGCCCGCCAAGCCTACCACAACCCCTGCCCCT
AGACCTCCTACCCCAGCCCCTACAATCGCCAGCCAGCCTC
TGTCTCTGAGGCCCGAGGCTTCTAGACCAGCTGCTGGCGG
AGCCGTGCACACCAGAGGACTGGACAAGCCCTTCTGGGTG
CTGGTGGTCGTGGGCGGAGTGCTGGCCTGTTATAGCCTGC
TCGTGACAGTGGCCTTCATCATCTTTTGGGTGCGCGTGAA
GTTCAGCCGCAGCGCCGATGCCCCTGCCTATCAGCAGGGA
CAGAACCAGCTGTACAACGAGCTGAACCTGGGCAGACGGG
AAGAGTACGACGTGCTGGACAAGAGAAGAGGCCGGGACCC
TGAGATGGGCGGCAAGCCCAGAAGAAAGAACCCCCAGGAA
GGCCTGTATAACGAACTGCAGAAAGACAAGATGGCCGAGG
CCTACAGCGAGATCGGCATGAAGGGCGAACGGCGGAGAGG
CAAGGGCCACGATGGACTGTATCAGGGCCTGAGCACCGCC
ACCAAGGACACCTATGACGCCCTGCACATGCAGGCTCTGC
CCCCTCGCTGAAATTCATCGACGTTAACTATTCTAG
CART5dual nucleic acid sequence
(SEQ ID NO: 30)
GGTGTCGTGAGCGGCCGCTGAACTGGCCACCATGTGGCTG
CAGTCTCTGCTGCTGCTGGGCACCGTGGCCTGTAGCATCA
GCGAGATCGTGCTGACCCAGAGCCCTGGCTCTCTGGCTGT
GTCTCCTGGCGAGCGCGTGACCATGAGCTGCAAGAGCAGC
CAGAGCGTGTTCTTCAGCAGCTCCCAGAAGAACTACCTGG
CCTGGTATCAGCAGATCCCCGGCCAGAGCCCCAGACTGCT
GATCTACTGGGCCAGCACCAGAGAAAGCGGCGTGCCCGAT
AGATTCACCGGCAGCGGCTCTGGCACCGACTTCACCCTGA
CAATCAGCAGCGTGCAGCCCGAGGACCTGGCCATCTACTA
CTGCCACCAGTACCTGAGCAGCCGGACCTTTGGCCAGGGC
ACCAAGCTGGAAATCAAGCGGGGCAGCACAAGCGGCAGCG
GAAAGCCTGGATCTGGCGAGGGCTCTACCAAGGGCCAGGT
GCAGCTGCAGCAGCCTGGCGCCGAAGTCGTGAAACCTGGC
GCCTCCGTGAAGATGTCCTGCAAGGCCAGCGGCTACACCT
TCACCAGCTACTACATCCACTGGATCAAGCAGACCCCTGG
ACAGGGCCTGGAATGGGTGGGAGTGATCTACCCCGGCAAC
GACGACATCAGCTACAACCAGAAGTTCCAGGGCAAGGCCA
CCCTGACCGCCGACAAGTCTAGCACCACCGCCTACATGCA
GCTGTCCAGCCTGACCAGCGAGGACAGCGCCGTGTACTAC
TGCGCCAGAGAAGTGCGGCTGCGGTACTTCGATGTGTGGG
GCCAGGGAACCACCGTGACCGTGTCTAGCGCCCTGAGCAA
CAGCATCATGTACTTCAGCCACTTCGTGCCCGTGTTTCTG
CCCGCCAAGCCTACCACAACCCCTGCCCCTAGACCTCCTA
CCCCAGCCCCTACAATCGCCAGCCAGCCTCTGTCTCTGAG
GCCCGAGGCTTCTAGACCAGCTGCTGGCGGAGCCGTGCAC
ACCAGAGGACTGGACAAGCCCTTCTGGGTGCTGGTGGTCG
TGGGCGGAGTGCTGGCCTGTTACAGCCTGCTCGTGACAGT
GGCCTTCATCATCTTTTGGGTGCGCAGCAAGCGGTCTAGA
CTGCTGCACAGCGACTACATGAACATGACCCCCAGAAGGC
CAGGCCCCACCCGGAAGCACTATCAGCCTTACGCCCCTCC
CAGAGACTTCGCCGCCTACAGAAGCTGAAATTCATCGACG
TTAACTATTCTAG
CART6 nucleic acid sequence
(SEQ ID NO: 31)
GGTGTCGTGAGCGGCCGCTGAACTGGCCACCATGTGGCTG
CAGTCTCTGCTGCTGCTGGGCACCGTGGCCTGCAGCATCA
GCATCCAGATGACCCAGACCACCAGCAGCCTGAGCGCCAG
CCTGGGCGATAGAGTGACCATCAGCTGCAGAGCCAGCCAG
GACATCAGCAAGTACCTGAACTGGTATCAGCAGAAACCCG
ACGGCACCGTGAAGCTGCTGATCTACCACACCAGCAGACT
GCACAGCGGCGTGCCCTCTAGATTTTCCGGCAGCGGCTCC
GGCACCGACTACAGCCTGACCATCTCCAACCTGGAACAGG
AAGATATCGCTACCTACTTCTGTCAGCAAGGCAACACCCT
GCCCTACACCTTCGGCGGAGGCACCAAGCTGGAAATCGGC
AGCACAAGCGGCTCTGGCAAGCCTGGATCTGGCGAGGGCT
CTACCAAGGGCCTGCAGGAATCTGGCCCTGGACTGGTGGC
CCCTAGCCAGAGCCTGTCTGTGACCTGTACCGTGTCCGGC
GTGTCCCTGCCTGACTATGGCGTGTCCTGGATCAGACAGC
CCCCCAGAAAGGGCCTGGAATGGCTGGGAGTGATCTGGGG
CAGCGAGACAACCTACTACAACAGCGCCCTGAAGTCCCGG
CTGACCATCATCAAGGACAACTCCAAGAGCCAGGTGTTCC
TGAAGATGAACAGCCTGCAGACCGACGACACCGCCATCTA
CTACTGCGCCAAGCACTACTACTACGGCGGCAGCTACGCC
ATGGACTACTGGGGCCAGGGCACAAGCGTGACCGTGTCTG
CCCTGAGCAACAGCATCATGTACTTCAGCCACTTCGTGCC
CGTGTTTCTGCCCGCCAAGCCTACCACAACCCCTGCCCCT
AGACCTCCTACCCCAGCCCCTACAATCGCCAGCCAGCCTC
TGTCTCTGAGGCCCGAGGCTTCTAGACCAGCTGCTGGCGG
AGCCGTGCACACCAGAGGACTGGACAAGCCCTTCTGGGTG
CTGGTGGTCGTGGGCGGAGTGCTGGCCTGTTATAGCCTGC
TCGTGACAGTGGCCTTCATCATCTTTTGGGTGCGCAGCAA
GCGGAGCCGGCTGCTGCACTCCGACTACATGAACATGACC
CCCAGACGGCCAGGCCCCACCCGGAAACACTATCAGCCTT
ACGCCCCTCCCAGAGACTTCGCCGCCTACCGGTCCAGAGT
GAAGTTCAGCAGATCCGCCGACGCCCCTGCCTATCAGCAG
GGACAGAACCAGCTGTACAACGAGCTGAACCTGGGCAGAC
GGGAAGAGTACGACGTGCTGGACAAGAGAAGAGGCCGGGA
CCCTGAGATGGGCGGCAAGCCCAGAAGAAAGAACCCCCAG
GAAGGCCTGTATAACGAACTGCAGAAAGACAAGATGGCCG
AGGCCTACAGCGAGATCGGCATGAAGGGCGAACGGCGGAG
AGGCAAGGGCCACGATGGACTGTATCAGGGCCTGAGCACC
GCCACCAAGGACACCTATGACGCCCTGCACATGCAGGCTC
TGCCCCCTCGCTGAAATTCATCGACGTTAACTATTCTAG
CART7 nucleic acid sequence
(SEQ ID NO: 32)
GGTGTCGTGAGCGGCCGCTGAACTGGCCACCATGTGGCTG
CAGTCTCTGCTGCTGCTGGGCACCGTGGCCTGTAGCATCA
GCGAGATCGTGCTGACCCAGAGCCCTGGCTCTCTGGCTGT
GTCTCCTGGCGAGCGCGTGACCATGAGCTGCAAGAGCAGC
CAGAGCGTGTTCTTCAGCAGCTCCCAGAAGAACTACCTGG
CCTGGTATCAGCAGATCCCCGGCCAGAGCCCCAGACTGCT
GATCTACTGGGCCAGCACCAGAGAAAGCGGCGTGCCCGAT
AGATTCACCGGCAGCGGCTCTGGCACCGACTTCACCCTGA
CAATCAGCAGCGTGCAGCCCGAGGACCTGGCCATCTACTA
CTGCCACCAGTACCTGAGCAGCCGGACCTTTGGCCAGGGC
ACCAAGCTGGAAATCAAGCGGGGCAGCACAAGCGGCAGCG
GAAAGCCTGGATCTGGCGAGGGCTCTACCAAGGGCCAGGT
GCAGCTGCAGCAGCCTGGCGCCGAAGTCGTGAAACCTGGC
GCCTCCGTGAAGATGTCCTGCAAGGCCAGCGGCTACACCT
TCACCAGCTACTACATCCACTGGATCAAGCAGACCCCTGG
ACAGGGCCTGGAATGGGTGGGAGTGATCTACCCCGGCAAC
GACGACATCAGCTACAACCAGAAGTTCCAGGGCAAGGCCA
CCCTGACCGCCGACAAGTCTAGCACCACCGCCTACATGCA
GCTGTCCAGCCTGACCAGCGAGGACAGCGCCGTGTACTAC
TGCGCCAGAGAAGTGCGGCTGCGGTACTTCGATGTGTGGG
GCCAGGGAACCACCGTGACCGTGTCCAGCATCGAAGTGAT
GTACCCCCCTCCCTACCTGGACAACGAGAAGTCCAACGGC
ACCATCATCCACGTGAAGGGCAAGCACCTGTGCCCCAGCC
CTCTGTTTCCTGGCCCTAGCAAGCCCTTCTGGGTGCTGGT
GGTCGTGGGCGGAGTGCTGGCCTGTTACAGCCTGCTCGTG
ACAGTGGCCTTCATCATCTTTTGGGTGCGCAGCAAGCGGT
CTAGACTGCTGCACAGCGACTACATGAACATGACCCCCAG
AAGGCCAGGCCCCACCCGGAAGCACTATCAGCCTTACGCC
CCTCCCAGAGACTTCGCCGCCTACCGGTCCAGAGTGAAGT
TCAGCAGAAGCGCCGACGCCCCTGCCTATCAGCAGGGCCA
GAACCAGCTGTACAACGAGCTGAACCTGGGCAGACGGGAA
GAGTACGACGTGCTGGACAAGCGGAGAGGCAGGGACCCTG
AGATGGGCGGCAAGCCCAGACGGAAGAACCCTCAGGAAGG
CCTGTATAACGAACTGCAGAAAGACAAGATGGCCGAGGCC
TACTCCGAGATCGGCATGAAGGGCGAGCGGAGAAGAGGCA
AGGGCCACGATGGACTGTACCAGGGCCTGAGCACCGCCAC
CAAGGACACCTATGACGCCCTGCACATGCAGGCCCTGCCC
CCCAGATGAAATTCATCGACGTTAACTATTCTAG

Furthermore, also contemplated herein are immune cells expressing chimeric receptors that target EMR2 in addition to chimeric receptors that target CD33 in AML patients. This can be accomplished by two different approaches: 1) generating immune cells expressing anti-CD33 chimeric receptors and immune cells expressing anti-EMR2 chimeric receptors separately and infusing the patient with both types of immune cells separately; or 2) generating immune cells that target both CD33 and EMR2 simultaneously (Kakarla et al., Cancer (2014) 2:151).

Any of the chimeric receptors described herein can be prepared by routine methods, such as recombinant technology. Methods for preparing the chimeric receptors herein involve generation of a nucleic acid that encodes a polypeptide comprising each of the domains of the chimeric receptors, including the antigen-binding fragment and optionally, the hinge domain, the transmembrane domain, at least one co-stimulatory signaling domain, and the cytoplasmic signaling domain. In some embodiments, a nucleic acid encoding each of the components of chimeric receptor are joined together using recombinant technology.

Sequences of each of the components of the chimeric receptors may be obtained via routine technology, e.g., PCR amplification from any one of a variety of sources known in the art. In some embodiments, sequences of one or more of the components of the chimeric receptors are obtained from a human cell. Alternatively, the sequences of one or more components of the chimeric receptors can be synthesized. Sequences of each of the components (e.g., domains) can be joined directly or indirectly (e.g., using a nucleic acid sequence encoding a peptide linker) to form a nucleic acid sequence encoding the chimeric receptor, using methods such as PCR amplification or ligation. Alternatively, the nucleic acid encoding the chimeric receptor may be synthesized. In some embodiments, the nucleic acid is DNA. In other embodiments, the nucleic acid is RNA.

Mutation of one or more residues within one or more of the components of the chimeric receptor (e.g., the antigen-binding fragment), prior to or after joining the sequences of each of the components. In some embodiments, one or more mutations in a component of the chimeric receptor may be made to modulate (increase or decrease) the affinity of the component for a target (e.g., the antigen-binding fragment for the target antigen) and/or modulate the activity of the component.

Any of the chimeric receptors described herein can be introduced into a suitable immune cell for expression via conventional technology. In some embodiments, the immune cells are T cells, such as primary T cells or T cell lines. Alternatively, the immune cells can be NK cells, such as established NK cell lines (e.g., NK-92 cells). In some embodiments, the immune cells are T cells that express CD8 (CD8⁺) or CD8 and CD4 (CD8⁺/CD4⁺). In some embodiments, the T cells are T cells of an established T cell line, for example, 293T cells or Jurkat cells.

Primary T cells may be obtained from any source, such as peripheral blood mononuclear cells (PBMCs), bone marrow, tissues such as spleen, lymph node, thymus, or tumor tissue. A source suitable for obtaining the type of immune cells desired would be evident to one of skill in the art. In some embodiments, the population of immune cells is derived from a human patient having a hematopoietic malignancy, such as from the bone marrow or from PBMCs obtained from the patient. In some embodiments, the population of immune cells is derived from a healthy donor. In some embodiments, the immune cells are obtained from the subject to whom the immune cells expressing the chimeric receptors will be subsequently administered. Immune cells that are administered to the same subject from which the cells were obtained are referred to as autologous cells, whereas immune cells that are obtained from a subject who is not the subject to whom the cells will be administered are referred to as allogeneic cells.

The type of host cells desired may be expanded within the population of cells obtained by co-incubating the cells with stimulatory molecules, for example, anti-CD3 and anti-CD28 antibodies may be used for expansion of T cells.

To construct the immune cells that express any of the chimeric receptor constructs described herein, expression vectors for stable or transient expression of the chimeric receptor construct may be constructed via conventional methods as described herein and introduced into immune host cells. For example, nucleic acids encoding the chimeric receptors may be cloned into a suitable expression vector, such as a viral vector in operable linkage to a suitable promoter. The nucleic acids and the vector may be contacted, under suitable conditions, with a restriction enzyme to create complementary ends on each molecule that can pair with each other and be joined with a ligase. Alternatively, synthetic nucleic acid linkers can be ligated to the termini of the nucleic acid encoding the chimeric receptors. The synthetic linkers may contain nucleic acid sequences that correspond to a particular restriction site in the vector. The selection of expression vectors/plasmids/viral vectors would depend on the type of host cells for expression of the chimeric receptors but should be suitable for integration and replication in eukaryotic cells.

A variety of promoters can be used for expression of the chimeric receptors described herein, including, without limitation, cytomegalovirus (CMV) intermediate early promoter, a viral LTR such as the Rous sarcoma virus LTR, HIV-LTR, HTLV-1 LTR, Maloney murine leukemia virus (MMLV) LTR, myeoloproliferative sarcoma virus (MPSV) LTR, spleen focus-forming virus (SFFV) LTR, the simian virus 40 (SV40) early promoter, herpes simplex tk virus promoter, elongation factor 1-alpha (EF1-α) promoter with or without the EF1-α intron. Additional promoters for expression of the chimeric receptors include any constitutively active promoter in an immune cell. Alternatively, any regulatable promoter may be used, such that its expression can be modulated within an immune cell.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes); versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9); and reporter gene for assessing expression of the chimeric receptor. Suitable vectors and methods for producing vectors containing transgenes are well known and available in the art. Examples of the preparation of vectors for expression of chimeric receptors can be found, for example, in US2014/0106449, herein incorporated by reference in its entirety.

In some embodiments, the chimeric receptor construct or the nucleic acid encoding said chimeric receptor is a DNA molecule. In some embodiments, chimeric receptor construct or the nucleic acid encoding said chimeric receptor is a DNA vector and may be electroporated to immune cells (see, e.g., Till et al., Blood (2012) 119(17): 3940-3950). In some embodiments, the nucleic acid encoding the chimeric receptor is an RNA molecule, which may be electroporated to immune cells.

Any of the vectors comprising a nucleic acid sequence that encodes a chimeric receptor construct described herein is also within the scope of the present disclosure. Such a vector may be delivered into host cells such as host immune cells by a suitable method. Methods of delivering vectors to immune cells are well known in the art and may include DNA, RNA, or transposon electroporation, transfection reagents such as liposomes or nanoparticles to delivery DNA, RNA, or transposons; delivery of DNA, RNA, or transposons or protein by mechanical deformation (see, e.g., Sharei et al. Proc. Natl. Acad. Sci. USA (2013) 110(6): 2082-2087); or viral transduction. In some embodiments, the vectors for expression of the chimeric receptors are delivered to host cells by viral transduction. Exemplary viral methods for delivery include, but are not limited to, recombinant retroviruses (see, e.g., PCT Publication Nos. WO 90/07936; WO 94/03622; WO 93/25698; WO 93/25234; WO 93/11230; WO 93/10218; WO 91/02805; U.S. Pat. Nos. 5,219,740 and 4,777,127; GB Patent No. 2,200,651; and EP Patent No. 0 345 242), alphavirus-based vectors, and adeno-associated virus (AAV) vectors (see, e.g., PCT Publication Nos. WO 94/12649, WO 93/03769; WO 93/19191; WO 94/28938; WO 95/11984 and WO 95/00655). In some embodiments, the vectors for expression of the chimeric receptors are retroviruses. In some embodiments, the vectors for expression of the chimeric receptors are lentiviruses. In some embodiments, the vectors for expression of the chimeric receptors are adeno-associated viruses.

In examples in which the vectors encoding chimeric receptors are introduced to the host cells using a viral vector, viral particles that are capable of infecting the immune cells and carry the vector may be produced by any method known in the art and can be found, for example in PCT Application No. WO 1991/002805^{Δ2}, WO 1998/009271 A1, and U.S. Pat. No. 6,194,191. The viral particles are harvested from the cell culture supernatant and may be isolated and/or purified prior to contacting the viral particles with the immune cells.

The methods of preparing host cells expressing any of the chimeric receptors described herein may comprise activating and/or expanding the immune cells ex vivo. Activating a host cell means stimulating a host cell into an activate state in which the cell may be able to perform effector functions (e.g., cytotoxicity). Methods of activating a host cell will depend on the type of host cell used for expression of the chimeric receptors. Expanding host cells may involve any method that results in an increase in the number of cells expressing chimeric receptors, for example, allowing the host cells to proliferate or stimulating the host cells to proliferate. Methods for stimulating expansion of host cells will depend on the type of host cell used for expression of the chimeric receptors and will be evident to one of skill in the art. In some embodiments, the host cells expressing any of the chimeric receptors described herein are activated and/or expanded ex vivo prior to administration to a subject.

In some embodiments, the agents targeting a lineage-specific cell-surface antigen(s) is an antibody-drug conjugate (ADC). As will be evident to one of ordinary skill in the art, the term “antibody-drug conjugate” can be used interchangeably with “immunotoxin” and refers to a fusion molecule comprising an antibody (or antigen-binding fragment thereof) conjugated to a toxin or drug molecule. Binding of the antibody to the corresponding antigen allows for delivery of the toxin or drug molecule to a cell that presents the antigen on its cell surface (e.g., target cell), thereby resulting in death of the target cell.

In some embodiments, the agent is an antibody-drug conjugate. In some embodiments, the antibody-drug conjugate comprises an antigen-binding fragment and a toxin or drug that induces cytotoxicity in a target cell. In some embodiments, the antibody-drug conjugate targets a type 2 antigen. In some embodiments, the antibody-drug conjugate targets CD33 or EMR2.

In some embodiments, the antigen-bind fragment of the antibody-drug conjugate has the same heavy chain CDRs as the heavy chain variable region provided by SEQ ID NO: 5 and the same light chain CDRS as the light chain variable region provided by SEQ ID NO: 7. In some embodiments, the antigen-bind fragment of the antibody-drug conjugate has the heavy chain variable region provided by SEQ ID NO: 5 and the same light chain variable region provided by SEQ ID NO: 7.

Toxins or drugs compatible for use in antibody-drug conjugate are well known in the art and will be evident to one of ordinary skill in the art. See, e.g., Peters et al., Biosci. Rep. (2015) 35(4): e00225; Beck et al., Nature Reviews Drug Discovery (2017) 16:315-337; Marin-Acevedo et al., J. Hematol. Oncol. (2018)11: 8; Elgundi et al., Advanced Drug Delivery Reviews (2017) 122: 2-19.

In some embodiments, the antibody-drug conjugate may further comprise a linker (e.g., a peptide linker, such as a cleavable linker) attaching the antibody and drug molecule. Examples of antibody-drug conjugates include, without limitation, brentuximab vedotin, glembatumumab vedotin/CDX-011, depatuxizumab mafodotin/ABT-414, PSMA ADC, polatuzumab vedotin/RG7596/DCDS4501A, denintuzumab mafodotin/SGN-CD19A, AGS-16C3F, CDX-014, RG7841/DLYE5953A, RG7882/DMUC406A, RG7986/DCDSO780A, SGN-LIV1A, enfortumab vedotin/ASG-22ME, AG-15ME, AGS67E, telisotuzumab vedotin/ABBV-399, ABBV-221, ABBV-085, GSK-2857916, tisotumab vedotin/HuMax-TF-ADC, HuMax-Axl-ADC, pinatuzumab veodtin/RG7593/DCDT2980S, lifastuzumab vedotin/RG7599/DNIB0600A, indusatumab vedotin/MLN-0264/TAK-264, vandortuzumab vedotin/RG7450/DSTP3086S, sofituzumab vedotin/RG7458/DMUC5754A, RG7600/DMOT4039A, RG7336/DEDN6526A, ME1547, PF-06263507/ADC 5T4, trastuzumab emtansine/T-DM1, mirvetuximab soravtansine/IMGN853, coltuximab ravtansine/SAR3419, naratuximab emtansine/IMGN529, indatuximab ravtansine/BT-062, anetumab ravtansine/BAY 94-9343, SAR408701, SAR428926, AMG 224, PCA062, HKT288, LY3076226, SAR566658, lorvotuzumab mertansine/IMGN901, cantuzumab mertansine/SB-408075, cantuzumab ravtansine/IMGN242, laprituximab emtansine/IMGN289, IMGN388, bivatuzumab mertansine, AVE9633, BIIB015, MLN2704, AMG 172, AMG 595, LOP 628, vadastuximab talirine/SGN-CD33A, SGN-CD70A, SGN-CD19B, SGN-CD123A, SGN-CD352A, rovalpituzumab tesirine/SC16LD6.5, SC-002, SC-003, ADCT-301/HuMax-TAC-PBD, ADCT-402, MED13726/ADC-401, IMGN779, IMGN632, gemtuzumab ozogamicin, inotuzumab ozogamicin/CMC-544, PF-06647263, CMD-193, CMB-401, trastuzumab duocarmazine/SYD985, BMS-936561/MDX-1203, sacituzumab govitecan/IMMU-132, labetuzumab govitecan/IMMU-130, DS-8201a, U3-1402, milatuzumab doxorubicin/IMMU-110/hLL1-DOX, BMS-986148, RC48-ADC/hertuzumab-vc-MMAE, PF-06647020, PF-06650808, PF-06664178/RN927C, lupartumab amadotin/BAY1129980, aprutumab ixadotin/BAY1187982, ARX788, AGS62P1, XMT-1522, AbGn-107, MEDI4276, DSTA4637S/RG7861. In one example, the antibody-drug conjugate is gemtuzumab ozogamicin.

In some embodiments, binding of the antibody-drug conjugate to the epitope of the lineage-specific cell-surface protein induces internalization of the antibody-drug conjugate, and the drug (or toxin) may be released intracellularly. In some embodiments, binding of the antibody-drug conjugate to the epitope of a lineage-specific cell-surface protein induces internalization of the toxin or drug, which allows the toxin or drug to kill the cells expressing the lineage-specific protein (target cells). In some embodiments, binding of the antibody-drug conjugate to the epitope of a lineage-specific cell-surface protein induces internalization of the toxin or drug, which may regulate the activity of the cell expressing the lineage-specific protein (target cells). The type of toxin or drug used in the antibody-drug conjugates described herein is not limited to any specific type.

An ADC described herein may be used as a follow-on treatment to subjects who have been undergone the combined therapy as described herein.

### Hematopoietic Cells Deficient in One or More Lineage-Specific Cell-Surface Antigen(s)

The present disclosure also provides hematopoietic cells such as hematopoietic stem cells (HSCs) and/or hematopoietic progenitor cells (HPCs) that have been genetically modified to be deficient in a lineage-specific cell-surface antigen (e.g., CD33). In some embodiments, the hematopoietic cells such as hematopoietic stem cells (HSCs) and/or hematopoietic progenitor cells (HPCs) that have been genetically modified to be deficient in a lineage-specific cell-surface antigen (e.g., CD33) and at least one additional lineage-specific cell-surface antigen (e.g., EMR2).

In some embodiments, a CRISPR-based base editor system has been used to genetically modify the cells and reduce off target effects and undesirable and debilitating immunosuppressive side effects. In some embodiments, one CRISPR-based base editor system is used to modify a cell to be deficient in a lineage-specific cell-surface antigen and a second CRISPR-based base editor system is used to modify the cell to be deficient in an at least one additional lineage-specific cell-surface antigen. Shown herein is the successful use of CRISPR-based base editor systems to modify cells to be deficient in more than one lineage-specific cell-surface antigen. In some embodiments, more than one additional lineage-specific cell-surface antigens can be deleted or inhibited in the same cell using multiple CRISPR-based base editor systems.

Shown herein is the use of a CRISPR-based base editor system to which results in a specific nucleotide substitution in an endogenous gene encoding the lineage-specific cell-surface antigen. In some embodiments, the nucleotide substitution is within a sequence encoding a splice element, wherein the nucleotide substitution results in an alternative splicing of a transcript encoded by the gene. In some embodiments, the CRISPR-based base editor system targets a splice element in an endogenous gene, wherein the CRISPR-based base editor system results in an alternative splicing of a transcript encoded by the gene. In some embodiments, the alternative splicing causes an exon encoding an epitope to be skipped. In some embodiments, the alternative splicing causes an exon encoding an epitope to be extended. In some embodiments, the alternative splicing induces an early codon termination. In some embodiments, the splice element is a splice donor, a splice acceptor, a splice enhancer or a splice acceptor. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”. In some embodiments, the epitope is targeted by a therapeutic or immunotherapeutic agent.

The CRISPR-based base editor systems described herein comprise a catalytically impaired Cas protein fused to a DNA-modifying enzyme, i.e., Cas9 nickase fused to a cytosine or adenosine deaminase (base editor), and a single guide RNA. Each CRISPR-based base editor complex can bind to the lineage specific antigen polynucleotide and allow the substitution of one or more nucleotides, thereby modifying the polynucleotide. More than one CRISPR-based base editor system can be used to modify more than one lineage-specific cell-surface antigen.

In some embodiments, the CRISPR-based base editor system comprises a Cas nickase fused to a cytosine base editor. In some embodiments, the CRISPR-based base editor is a cytosine base editor. In some embodiments, the cytosine base editor is BE4max. In some embodiments, the CRISPR-based base editor system targets CD33 and the gRNA is chosen from the group consisting of SEQ ID NOs: 1 and 2.

In some embodiments, the CRISPR-based base editor is an adenosine base editor. In some embodiments, the cytosine base editor is ABE8e. In some embodiments, the CRISPR-based base editor system targets CD33 and the gRNA is SEQ ID NO: 3.

In some embodiments, a splice acceptor or exonic splicing enhancer site in exon 2 of CD33 is altered. In some embodiments, the nucleotide sequence of the intron 1/exon 2 junction of CD33 is altered. In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”. In some embodiments, the gRNA sequence hybridizes to a portion of the nucleotide sequence encoding CD33. In some embodiments, the gRNA sequence targets a splice acceptor or exonic splicing enhancer site in exon 2 of the nucleotide sequence encoding CD33. In some embodiments, the gRNA sequence targets a SNP of CD33, rs12459419. In some embodiments, the gRNA sequence targets the intron 1/exon 2 junction of CD33. In some embodiments, the gRNA sequence targets a nucleotide sequence comprising SEQ ID NO: 37.

In some embodiments, the CRISPR-based base editor is an adenosine base editor. In some embodiments, the cytosine base editor is ABE8e. In some embodiments, the CRISPR-based base editor system targets EMR2 and the gRNA is SEQ ID NO: 4. In some embodiments, the CRISPR-based base editor system targets EMR2 and the gRNA is SEQ ID NO: 46. In some embodiments, the CRISPR-based base editor system targets EMR2 and the gRNA is SEQ ID NO: 47

In some embodiments, a splice donor site in exon 13 of EMR2 is altered. In some embodiments, the nucleotide sequence of the intron 12/exon 13 junction of EMR2 is altered.

In some embodiments, the nucleotide substitution is a “C” to an “T”. In some embodiments, the nucleotide substitution is a “G” to a “A”. In some embodiments, the nucleotide substitution is a “A” to a “G”. In some embodiments, the nucleotide substitution is a “T” to a “C”. In some embodiments, the gRNA sequence hybridizes to a portion of the nucleotide sequence encoding EMR2. In some embodiments, the gRNA sequence targets a splice donor site in exon 13 of the nucleotide sequence encoding EMR2. In some embodiments, the gRNA sequence targets the intron 12/exon 13 junction of EMR2. In some embodiments, the gRNA sequence targets a nucleotide sequence comprising SEQ ID NO: 40.

In some embodiments, all of the foregoing described CRISPR-based base editor systems can comprise a base editor protein and the gRNA in a ribonucleoprotein (RNP) based delivery system.

In some embodiments, the hematopoietic cells are HSCs, HPCs, or a combination thereof, referred to herein as “HSPCs” (“hematopoietic stem and/or progenitor cells”). In some embodiments, a population of cells described herein comprises a plurality of hematopoietic stem cells; in some embodiments, a population of cells described herein comprises a plurality of hematopoietic progenitor cells; and in some embodiments, a population of cells described herein comprises a plurality of hematopoietic stem cells and a plurality of hematopoietic progenitor cells. HSCs are capable of giving rise to both myeloid and lymphoid progenitor cells that further give rise to myeloid cells (e.g., monocytes, macrophages, neutrophils, basophils, dendritic cells, erythrocytes, platelets, etc) and lymphoid cells (e.g., T cells, B cells, NK cells), respectively. HSCs are characterized by the expression of the cell surface marker CD34 (e.g., CD34⁺), which can be used for the identification and/or isolation of HSCs, and absence of cell surface markers associated with commitment to a cell lineage. Therefore, in some embodiments, the HSCs are CD34⁺.

In some embodiments, the HSCs are obtained from a subject, such as a mammalian subject. In some embodiments, the mammalian subject is a non-human primate, a rodent (e.g., mouse or rat), a bovine, a porcine, an equine, or a domestic animal. In some embodiments, the HSCs are obtained from a human patient, such as a human patient having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

HSCs may be obtained from any suitable source using convention means known in the art. In some embodiments, HSCs are obtained from a sample from a subject, such as bone marrow sample or from a blood sample. Alternatively, or in addition, HSCs may be obtained from an umbilical cord. In some embodiments, the HSCs are from bone marrow or peripheral blood mononuclear cells (PBMCs). In general, bone marrow cells may be obtained from iliac crest, femora, tibiae, spine, rib or other medullary spaces of a subject. Bone marrow may be taken out of the patient and isolated through various separations and washing procedures known in the art. An exemplary procedure for isolation of bone marrow cells comprises the following steps: a) extraction of a bone marrow sample; b) centrifugal separation of bone marrow suspension in three fractions and collecting the intermediate fraction, or buffycoat; c) the buffycoat fraction from step (b) is centrifuged one more time in a separation fluid, commonly Ficoll™, and an intermediate fraction which contains the bone marrow cells is collected; and d) washing of the collected fraction from step (c) for recovery of re-transfusable bone marrow cells.

HSCs typically reside in the bone marrow but can be mobilized into the circulating blood by administering a mobilizing agent in order to harvest HSCs from the peripheral blood. In some embodiments, the subject from which the HSCs are obtained is administered a mobilizing agent, such as granulocyte colony-stimulating factor (G-CSF). The number of the HSCs collected following mobilization using a mobilizing agent is typically greater than the number of cells obtained without use of a mobilizing agent. In some embodiments, the HSCs are peripheral blood HSCs.

In some embodiments, a sample is obtained from a subject and is then enriched for a desired cell type. For example, PBMCs and/or CD34⁺ hematopoietic cells can be isolated from blood as described herein. Cells can also be isolated from other cells, for example by isolation and/or activation with an antibody binding to an epitope on the cell surface of the desired cell type. Another method that can be used includes negative selection using antibodies to cell surface markers to selectively enrich for a specific cell type without activating the cell by receptor engagement.

Populations of HSC can be expanded prior to or after genetically engineering the HSC to become deficient in a lineage specific cell-surface antigen. The cells may be cultured under conditions that comprise an expansion medium comprising one or more cytokines, such as stem cell factor (SCF), Flt-3 ligand (Flt3L), thrombopoietin (TPO), Interleukin 3 (IL-3), or Interleukin 6 (IL-6). The cell may be expanded for about 1, 2, 3, 4, 5, 6, 7, 8, 9, 10, 11, 12, 13, 14, 15, 16, 17, 18, 19, 20, 21, 22, 23, 24, 25 days or any range necessary. In some embodiments, the HSC are expanded after isolation of a desired cell population (e.g., CD34⁺/CD33⁻) from a sample obtained from a subject and prior to genetic engineering. In some embodiments, the HSC are expanded after genetic engineering, thereby selectively expanding cells that have undergone the genetic modification and are deficient in a lineage-specific cell-surface antigen. In some embodiments, a cell (“a clone”) or several cells having a desired characteristic (e.g., phenotype or genotype) following genetic modification may be selected and independently expanded.

In some embodiments, the hematopoietic cells are genetically engineered to be deficient in (e.g., do not express) a lineage-specific antigen cell-surface (e.g., CD33). In some embodiments, the hematopoietic cells are genetically engineered to be deficient in (e.g., do not express) a lineage-specific cell-surface antigen (e.g., CD33) and at least one additional lineage-specific cell-surface antigen. In some embodiments, the hematopoietic cells are genetically engineered to be deficient in the same lineage-specific cell-surface antigen(s) that is targeted by the agent(s). As used herein, a hematopoietic cell is considered to be deficient in a lineage-specific cell-surface antigen(s) if hematopoietic cell has substantially reduced expression of the lineage-specific cell-surface antigen(s) as compared to a naturally-occurring hematopoietic cell of the same type as the genetically engineered hematopoietic cell (e.g., is characterized by the presence of the same cell surface markers, such as CD34). In some embodiments, the hematopoietic cell has no detectable expression of the lineage-specific cell-surface antigen(s) (e.g., does not express the lineage-specific cell-surface antigen(s)). The expression level of a lineage-specific cell-surface antigen can be assessed by any means known in the art. For example, the expression level of a lineage-specific cell-surface antigen can be assessed by detecting the antigen with an antigen-specific antibody (e.g., flow cytometry methods, Western blotting).

In some embodiments, the expression of the lineage-specific cell-surface antigen(s) on the genetically engineered hematopoietic cell is compared to the expression of the lineage-specific cell-surface antigen(s) on a naturally occurring hematopoietic cell (e.g., a wild-type counterpart). In some embodiments, the genetic engineering results in a reduction in the expression level of the lineage-specific cell-surface antigen(s) by at least about 50%, 60%, 70%, 80%, 90%, 91%, 92%, 93%, 94%, 95%, 96%, 97%, 98%, or 99% as compared to the expression of the lineage-specific cell-surface antigen(s) on a naturally occurring hematopoietic cell. That is, in some embodiments, the genetically engineered hematopoietic cell expresses less than about 20%, 19%, 18%, 17%, 16%, 15%, 14%, 13%, 12%, 11%, 10%, 9%, 8%, 7%, 6%, 5%, 4%, 3%, 2%, or 1% of the lineage-specific cell-surface antigen(s) (e.g., CD33) as compared to a naturally occurring hematopoietic cell (e.g., a wild-type counterpart).

In some embodiments, the genetic engineering results in a reduction in the expression level of a wild-type lineage-specific cell-surface antigen(s) (e.g., CD33) by at least about 50%, 60%, 70%, 80%, 90%, 91%, 92%, 93%, 94%, 95%, 96%, 97%, 98%, or 99% as compared to the expression of the level of the wild-type lineage-specific cell-surface antigen(s) on a naturally occurring hematopoietic cell. That is, in some embodiments, the genetically engineered hematopoietic cell expresses less than about 20%, 19%, 18%, 17%, 16%, 15%, 14%, 13%, 12%, 11%, 10%, 9%, 8%, 7%, 6%, 5%, 4%, 3%, 2%, or 1% of a wild-type lineage-specific cell-surface antigen(s) (e.g., CD33) as compared to a naturally occurring hematopoietic cell (e.g., a wild-type counterpart).

In some embodiments, the hematopoietic cell is deficient in the whole endogenous gene encoding the lineage-specific cell-surface antigen(s). In some embodiments, the whole endogenous gene encoding the lineage-specific cell-surface antigen(s) has been deleted. In some embodiments, the hematopoietic cell comprises a portion of endogenous gene encoding the lineage-specific cell-surface antigen(s). In some embodiments, the hematopoietic cell expressing a portion (e.g. a truncated protein) of the lineage-specific cell-surface antigen(s). In other embodiments, a portion of the endogenous gene encoding the lineage-specific cell-surface antigen(s) has been deleted. In some embodiments, at least 10%, 20%, 30%, 40%, 50%, 60%, 70% or more of the gene encoding the lineage-specific cell-surface antigen(s) has been deleted.

In some embodiments, the expression of an epitope encoded by an exon of the lineage-specific cell-surface antigen(s) on the genetically engineered hematopoietic cell is compared to the expression the epitope of the lineage-specific cell-surface antigen(s) on a naturally occurring hematopoietic cell (e.g., a wild-type counterpart). In some embodiments, the genetic engineering results in a reduction in the expression level of the epitope of the lineage-specific cell-surface antigen(s) by at least about 50%, 60%, 70%, 80%, 90%, 91%, 92%, 93%, 94%, 95%, 96%, 97%, 98%, or 99% as compared to the expression of the epitope of the lineage-specific cell-surface antigen(s) on a naturally occurring hematopoietic cell. That is, in some embodiments, the genetically engineered hematopoietic cell expresses less than about 20%, 19%, 18%, 17%, 16%, 15%, 14%, 13%, 12%, 11%, 10%, 9%, 8%, 7%, 6%, 5%, 4%, 3%, 2%, or 1% of the epitope of the lineage-specific cell-surface antigen(s) (e.g., CD33) as compared to a naturally occurring hematopoietic cell (e.g., a wild-type counterpart).

In some embodiments, a substitution of one or more nucleotides is made in an epitope encoded by an exon of the endogenous gene.

As will be appreciated by one of ordinary skill in the art, a portion of the nucleotide sequence encoding the lineage-specific cell-surface antigen(s) may be deleted or one or more non-coding sequences, such that the hematopoietic cell is deficient in the antigen(s) (e.g., has substantially reduced expression of the antigen(s)).

In some embodiments, the lineage-specific cell-surface antigen is CD33. The predicted structure of CD33 includes two immunoglobulin domains, an IgV domain and an IgC2 domain. In some embodiments, exon 2 of CD33 is deleted. In some embodiments, an altered splice acceptor or exonic splicing enhancer in the exon 2 of an endogenous CD33 gene is altered and the alteration causes a reduced expression level of an epitope encoded by exon 2 of CD33 as compared with a wild-type counterpart cell.

In some embodiments, the at least one additional lineage-specific cell-surface antigen is EMR2. In some embodiments, exon 13 of EMR2 is deleted. In some embodiments, an altered splice donor in the exon 13 of an endogenous EMR2 gene is altered and the alteration causes a reduced expression level of an epitope encoded by exon 13 of EMR2 as compared with a wild-type counterpart cell. In some embodiments, the alternative splicing induces an early codon termination and production of a mutated or truncated EMR2 as compared with a wildtype counterpart cell.

Any of the genetically engineering hematopoietic cells, such as HSCs, that are deficient or altered in one or more cell-surface lineage-specific antigens can be prepared by a routine method or by a method described herein. In some embodiments, the genetic engineering is performed using genome editing. As used herein, “genome editing” refers to a method of modifying the genome, including any protein-coding or non-coding nucleotide sequence, of an organism to knock out the expression of a target gene.

In one aspect of the present disclosure, the replacement of the tumor cells by a modified population of normal cells is performed using normal cells in which a lineage-specific antigen is modified. Such modification may include the depletion or inhibition of any lineage specific antigen using a Clustered Regularly Interspaced Short Palindromic Repeats (CRISPR)-based base editor system.

CRISPR-Cas system has been successfully utilized to edit the genomes of various organisms, including, but not limited to bacteria, humans, fruit flies, zebra fish and plants. See, e.g., Jiang et al., Nature Biotechnology (2013) 31(3):233; Qi et al, Cell (2013) 5:1173; DiCarlo et al., Nucleic Acids Res. (2013) 7:4336; Hwang et al., Nat. Biotechnol (2013), 3:227); Gratz et al., Genetics (2013) 194:1029; Cong et al., Science (2013) 6121:819; Mali et al., Science (2013) 6121:823; Cho et al. Nat. Biotechnol (2013) 3: 230; and Jiang et al., Nucleic Acids Research (2013) 41(20):e188.

The present disclosure utilizes the CRISPR-based base editor system that hybridizes with a target sequence in a lineage specific antigen polynucleotide, where the CRISPR-based base editor system comprises a catalytically impaired Cas protein fused to a DNA-modifying enzyme, i.e., Cas9 nickase fused to a cytosine or adenosine deaminase (base editor), and a single guide RNA. The CRISPR-based base editor complex can bind to the lineage specific antigen polynucleotide and allow the substitution of one or more nucleotides, thereby modifying the polynucleotide.

The CRISPR-based base editor system of the present disclosure may bind to and/or cleave the region of interest within a cell-surface lineage-specific antigen in a coding or non-coding region, within or adjacent to the gene, such as, for example, a leader sequence, trailer sequence or intron, or within a non-transcribed region, either upstream or downstream of the coding region. The guide RNAs (gRNAs) used in the present disclosure may be designed such that the gRNA directs binding of the base editor protein-gRNA complexes to a pre-determined cleavage sites (target site) in a genome. The cleavage sites may be chosen so as to release a fragment that contains a region of unknown sequence, or a region containing a SNP, nucleotide insertion, nucleotide deletion, rearrangement, etc.

The guide RNAs (gRNAs) used in the present disclosure may be designed such that the gRNA directs binding of the base editor protein-gRNA complexes to a pre-determined target site in a genome. The target sites may be a region containing a SNP, nucleotide insertion, nucleotide deletion, rearrangement, etc.

The terms “gRNA,” “guide RNA” and “CRISPR guide sequence” may be used interchangeably throughout and refer to a nucleic acid comprising a sequence that determines the specificity of a base editor protein of a CRISPR-based base editor system. A gRNA hybridizes to (complementary to, partially or completely) a target nucleic acid sequence in the genome of a host cell. The gRNA or portion thereof that hybridizes to the target nucleic acid may be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In some embodiments, the gRNA sequence that hybridizes to the target nucleic acid is between 10-30 or between 15-25, nucleotides in length.

In addition to a sequence that binds to a target nucleic acid, in some embodiments, the gRNA also comprises a scaffold sequence. Expression of a gRNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gRNA may be referred to as a single guide RNA (sgRNA).

In some embodiments, the gRNA is modified, e.g., is chemically modified. The modified gRNA comprises at least one nucleotide having a modification to the chemical structure of at least one of the following: nucleobase, sugar, and phosphodiester linkage or backbone portion (e.g., nucleotide phosphates). Exemplary gRNA modifications will be evident to one of skill in the art and can be found, for example, in Lee et al., Elife (2017) May 2; 6 and U.S. Publication 2016/0289675. Additional suitable modifications include phosphorothioate backbone modification, 2′-O-Me-modified sugar, 2′F-modified sugar, replacement of the ribose sugar with the bicyclic nucleotide-cEt, 3′thioPACE (MSP), or any combination thereof. Suitable gRNA modifications are described, e.g., in Rahdar et al. PNAS (2015) 112 (51): E7110-E7117 and Hendel et al., Nat Biotechnol. (2015 September) 33(9): 985-989, each of which is incorporated herein by reference in its entirety.

In some embodiments, a gRNA described herein is chemically modified. For example, the gRNA may comprise one or more 2′-0 modified nucleotide, e.g., 2′-O-methyl nucleotide. In some embodiments, the gRNA comprises a 2′-0 modified nucleotide, e.g., 2′-O-methyl nucleotide at the 5′ end of the gRNA. In some embodiments, the gRNA comprises a 2′-0 modified nucleotide, e.g., 2′-O-methyl nucleotide at the 3′ end of the gRNA. In some embodiments, the gRNA comprises a 2′-O-modified nucleotide, e.g., 2′-O-methyl nucleotide at both the 5′ and 3′ ends of the gRNA. In some embodiments, the gRNA is 2′-O-modified, e.g., 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g., 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g., 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g., 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g., 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide.

In some embodiments, the gRNA may comprise one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous-modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O-modified and 3′phosphorous-modified, e.g., 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g., 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′phosphorothioate-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA may comprise one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O-modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g., 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g., 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g., 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, the gRNA comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, the gRNA comprises a thioPACE linkage. In some embodiments, the gRNA comprises a backbone in which one or more non-bridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

Chemical modifications of gRNAs are described, for example, in Hendel et al., Nature Biotech. (2015) 33(9), which is herein incorporated by reference in its entirety.

As used herein, a “scaffold sequence,” also referred to as a tracrRNA, refers to a nucleic acid sequence that recruits a Cas endonuclease to a target nucleic acid bound (hybridized) to a complementary gRNA sequence. Any scaffold sequence that comprises at least one stem loop structure and recruits an endonuclease may be used in the genetic elements and vectors described herein. Exemplary scaffold sequences will be evident to one of skill in the art and can be found, for example, in Jinek, et al. Science (2012) 337(6096):816-821, Ran, et al. Nature Protocols (2013) 8:2281-2308, PCT Application No. WO2014/093694, and PCT Application No. WO2013/176772.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence and recruit the endonuclease to the target nucleic acid.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid (see also U.S. Pat. No. 8,697,359, which is incorporated by reference for its teaching of complementarity of a gRNA sequence with a target polynucleotide sequence). It has been demonstrated that mismatches between a CRISPR guide sequence and the target nucleic acid near the 3′ end of the target nucleic acid may abolish nuclease cleavage activity (Upadhyay, et al. Genes Genome Genetics (2013) 3(12):2233-2238). In some embodiments, the gRNA sequence is at least 50%, 55%, 60%, 65%, 70%, 75%, 80%, 85%, 90%, 95%, 96%, 97%, 98%, 99%, or at least 100% complementary to the 3′ end of the target nucleic acid (e.g., the last 5, 6, 7, 8, 9, or 10 nucleotides of the 3′ end of the target nucleic acid).

The target nucleic acid is flanked on the 3′ side by a protospacer adjacent motif (PAM) that may interact with the endonuclease and be further involved in targeting the endonuclease activity to the target nucleic acid. It is generally thought that the PAM sequence flanking the target nucleic acid depends on the endonuclease and the source from which the endonuclease is derived. For example, for Cas9 endonucleases that are derived from Streptococcus pyogenes, the PAM sequence is NGG. For Cas9 endonucleases derived from Staphylococcus aureus, the PAM sequence is NNGRRT. For Cas9 endonucleases that are derived from Neisseria meningitidis, the PAM sequence is NNNNGATT. For Cas9 endonucleases derived from Streptococcus thermophilus, the PAM sequence is NNAGAA. For Cas9 endonuclease derived from Treponema denticola, the PAM sequence is NAAAAC. For a Cpf1 nuclease, the PAM sequence is TTN.

In some embodiments, genetically engineering a cell also comprises introducing a CRISPR-based base editor protein into the cell. In some embodiments, the CRISPR-based base editor and the nucleic acid encoding the gRNA are provided on the same nucleic acid (e.g., a vector). In some embodiments, the CRISPR-based base editor protein and the nucleic acid encoding the gRNA are provided on different nucleic acids (e.g., different vectors). Alternatively, or in addition, the CRISPR-based base editor may be provided or introduced into the cell in protein form. In some embodiments, the gRNA is complexed to the CRISPR-based base editor in protein form.

In some embodiments, the Cas endonuclease is a Cas9 enzyme or variant thereof. In some embodiments, the Cas9 endonuclease is derived from Streptococcus pyogenes (SpCas9), Staphylococcus aureus (SaCas9), Neisseria meningitidis (NmCas9), Streptococcus thermophilus, Campylobacter jejuni (CjCas9), or Treponema denticola. In some embodiments, the nucleotide sequence encoding the Cas endonuclease may be codon optimized for expression in a host cell. In some embodiments, the endonuclease is a Cas9 homolog or ortholog. In some embodiments, the nucleotide sequence encoding the Cas9 endonuclease is further modified to alter the activity of the protein. In some embodiments, the Cas9 endonuclease has been modified to inactivate one of the catalytic residues of the endonuclease, referred to as a “nickase” or “Cas9n”. Cas9 nickase endonucleases cleave one DNA strand of the target nucleic acid. See, e.g., Dabrowska et al. Frontiers in Neuroscience (2018) 12(75). It has been shown that one or more mutations in the RuvC and HNH catalytic domains of the enzyme may improve Cas9 efficiency. See, e.g., Sarai et al. Currently Pharma. Biotechnol. (2017) 18(13). In some embodiments, the Cas9 endonuclease is a catalytically inactive Cas9. For example, dCas9 contains mutations of catalytically active residues (D10 and H840) and does not have nuclease activity. Alternatively, or in addition, the Cas9 endonuclease may be fused to another protein or portion thereof. In some embodiments, dCas9 is fused to a repressor domain, such as a KRAB domain. In some embodiments, such dCas9 fusion proteins are used with the constructs described herein for multiplexed gene repression (e.g., CRISPR interference (CRISPRi)). In some embodiments, dCas9 is fused to an activator domain, such as VP64 or VPR. In some embodiments, such dCas9 fusion proteins are used with the constructs described herein for gene activation (e.g., CRISPR activation (CRISPRa)). In some embodiments, dCas9 is fused to an epigenetic modulating domain, such as a histone demethylase domain or a histone acetyltransferase domain. In some embodiments, dCas9 is fused to a LSD1 or p300, or a portion thereof. In some embodiments, the dCas9 fusion is used for CRISPR-based epigenetic modulation. In some embodiments, dCas9 or Cas9 is fused to a Fok1 nuclease domain. In some embodiments, Cas9 or dCas9 fused to a Fok1 nuclease domain is used for genome editing. In some embodiments, Cas9 or dCas9 is fused to a fluorescent protein (e.g., GFP, RFP, mCherry, etc.). In some embodiments, Cas9/dCas9 proteins fused to fluorescent proteins are used for labeling and/or visualization of genomic loci or identifying cells expressing the Cas endonuclease.

In some embodiments, the Cas endonuclease is modified to enhance specificity of the enzyme (e.g., reduce off-target effects, maintain robust on-target cleavage). In some embodiments, the Cas endonuclease is an enhanced specificity Cas9 variant (e.g., eSPCas9). See, e.g., Slaymaker et al. Science (2016) 351 (6268): 84-88. In some embodiments, the Cas endonuclease is a high fidelity Cas9 variant (e.g., SpCas9-HF1). See, e.g., Kleinstiver et al. Nature (2016) 529: 490-495.

Cas enzymes, such as Cas endonucleases, are known in the art and may be obtained from various sources and/or engineered/modified to modulate one or more activities or specificities of the enzymes. In some embodiments, the Cas enzyme has been engineered/modified to recognize one or more PAM sequence. In some embodiments, the Cas enzyme has been engineered/modified to recognize one or more PAM sequence that is different than the PAM sequence the Cas enzyme recognizes without engineering/modification. In some embodiments, the Cas enzyme has been engineered/modified to reduce off-target activity of the enzyme.

In some embodiments, the nucleotide sequence encoding the Cas endonuclease is modified further to alter the specificity of the endonuclease activity (e.g., reduce off-target cleavage, decrease the Cas endonuclease activity or lifetime in cells, increase homology-directed recombination and reduce non-homologous end joining). See, e.g., Komor et al. Cell (2017) 168: 20-36. In some embodiments, the nucleotide sequence encoding the Cas endonuclease is modified to alter the PAM recognition of the endonuclease. For example, the Cas endonuclease SpCas9 recognizes PAM sequence NGG, whereas relaxed variants of the SpCas9 comprising one or more modifications of the endonuclease (e.g., VQR SpCas9, EQR SpCas9, VRER SpCas9) may recognize the PAM sequences NGA, NGAG, NGCG. PAM recognition of a modified Cas endonuclease is considered “relaxed” if the Cas endonuclease recognizes more potential PAM sequences as compared to the Cas endonuclease that has not been modified. For example, the Cas endonuclease SaCas9 recognizes PAM sequence NNGRRT, whereas a relaxed variant of the SaCas9 comprising one or more modifications of the endonuclease (e.g., KKH SaCas9) may recognize the PAM sequence NNNRRT. In one example, the Cas endonuclease FnCas9 recognizes PAM sequence NNG, whereas a relaxed variant of the FnCas9 comprising one or more modifications of the endonuclease (e.g., RHA FnCas9) may recognize the PAM sequence YG. In one example, the Cas endonuclease is a Cpf1 endonuclease comprising substitution mutations S542R and K607R and recognize the PAM sequence TYCV. In one example, the Cas endonuclease is a Cpf1 endonuclease comprising substitution mutations S542R, K607R, and N552R and recognize the PAM sequence TATV. See, e.g., Gao et al. Nat. Biotechnol. (2017) 35(8): 789-792.

In some embodiments, more than one (e.g., 2, 3, or more) Cas endonucleases are used. In some embodiments, at least one of the Cas endonucleases is a Cas9 enzyme. In some embodiments, at least one of the Cas endonucleases is a Cpf1 enzyme. In some embodiments, at least one of the Cas9 endonucleases is derived from Streptococcus pyogenes. In some embodiments, at least one of the Cas9 endonuclease is derived from Streptococcus pyogenes and at least one Cas9 endonuclease is derived from an organism that is not Streptococcus pyogenes.

In some embodiments, the endonuclease is a base editor. Base editor endonuclease generally comprises a catalytically inactive Cas endonuclease fused to a function domain. See, e.g., Eid et al. Biochem. J. (2018) 475(11): 1955-1964; Rees et al. Nature Reviews Genetics (2018) 19:770-788.

CRISPR-based base editing systems generally comprise: a Cas nickase or Cas fused to a deaminase that makes the edit; a gRNA targeting Cas to a specific locus; and a target base for editing within the editing window specified by the Cas protein.

There are currently two classes of base editors, cytosine (CBEs) and adenine (ABEs). CBEs mediate a C to T change (or a G to A change on the opposite strand). ABEs make an A to G change (or a T to C change on the opposite strand). This only accounts for four of 12 possible changes.

The first cytosine base editor was made by coupling a cytidine deaminase with the inactive dCas9 (Komor et al., Nature (2016) 533:420-424). These fusions convert cytosine to uracil without cutting DNA. Uracil is then subsequently converted to thymine through DNA replication or repair. Fusing an inhibitor of uracil DNA glycosylase (UGI) to dCas9 prevents base excision repair which changes the U back to a C mutation. To increase base editing efficiency, a Cas nickase was used instead of dCas9. The resulting editor, BE3, nicks the unmodified DNA strand so that it appears “newly synthesized” to the cell. Thus, the cell repairs the DNA using the U-containing strand as a template, copying the base edit.

The fourth-generation base editors, BE4, reduce the undesired C->G or C->A conversions that can happen with earlier BEs. These byproducts likely resulted from excision by uracil N-glycosylase (UNG) during base excision repair. Adding a second copy of the UNG inhibitor, UGI, increases base editing product purity. The APOBEC1-Cas9n and Cas9n-UGI linkers were extended to improve product purity, and these three improvements represent the fourth generation of base editors. Compared to BE3, BE4 offers a 2.3 fold decrease in C->G and C->A products as well as a 2.3 fold decrease in indel formation.

To improve base editing efficiency for mammalian edits is to ensure the editors make it into the nucleus and that they are expressed well. The nuclear localization signals and codon usage of BE4 was improved to create BE4max and AncBE4max with a 4.2-6-fold improvement in editing efficiency (Koblan et al., Nat Biotechnol (2018) 36:843-846).

Adenine base editors convert adenine to inosine, resulting in an A to G change (Gaudelli et al., Nature (2017) 551:464-471). Creating an adenine base editor requires an additional step because there are no known DNA adenine deaminases. They used directed evolution to create one from the RNA adenine deaminase TadA.

Improvements have also been made to adenine base editor by improving nuclear localization and expression. In 2020, two papers were published describing additional ABEs evolved from ABE7.10 that have improved base editor targeting flexibility and specificity. In this first ABE8e was generated, which edits ˜590-fold faster than the TadA from ABE7.10 without increasing off-target activity (Richter et al., Nat Biotechnol (2020) 38:883-891).

Additionally, using ABE7.10 as a starting point, Gaudelli evolved the base editor into 40 new ABE8 variants (Gaudelli et al., Nat Biotechnol (2020) 38:892-900). Compared to ABE7.10, ABE8s resulted in 1.5-fold more editing at protospacer positions A5-A7 and 3.2-fold more editing at positions A3-A4 and A8-A10 at NGG PAM and 4.2-fold higher editing efficiency at non-NGG PAM variants compared to ABE7.10. ABE8s have an improved base editing capacity, even at sites previously difficult to target. ABE8s can achieve 98-99% target modification in primary T cells making them a promising tool for cell therapy applications.

In some embodiments, the catalytically inactive Cas endonuclease is dCas9. In some embodiments, the endonuclease comprises a dCas9 fused to one or more uracil glycosylase inhibitor (UGI) domains. In some embodiments, the endonuclease comprises a dCas9 fused to an adenine base editor (ABE), for example an ABE evolved from the RNA adenine deaminase TadA. In some embodiments, the endonuclease comprises a dCas9 fused to an ABE8e. In some embodiments, the endonuclease comprises a dCas9 fused to cytidine deaminase enzyme (e.g., APOBEC deaminase, pmCDA1, activation-induced cytidine deaminase (AID)). In some embodiments, the endonuclease comprised a dCas9 fused to a BE4max.

In some embodiments, the catalytically inactive Cas endonuclease is Cas9 nickase or Cas9n. In some embodiments, the endonuclease comprises a Cas9 nickase fused to one or more uracil glycosylase inhibitor (UGI) domains. In some embodiments, the endonuclease comprises a Cas9 nickase fused to an adenine base editor (ABE), for example an ABE evolved from the RNA adenine deaminase TadA. In some embodiments, the endonuclease comprises a Cas9 nickase fused to a ABE8e. In some embodiments, the endonuclease comprises a Cas9 nickase fused to cytidine deaminase enzyme (e.g., APOBEC deaminase, pmCDA1, activation-induced cytidine deaminase (AID)). In some embodiments, the endonuclease comprised a Cas9 nickase fused to a BE4max.

Examples of base editors include, without limitation, BE1, BE2, BE3, HF-BE3, BE4, BE4max, AncBE4max, BE4-Gam, YE1-BE3, EE-BE3, YE2-BE3, YEE-CE3, VQR-BE3, VRER-BE3, SaBE3, SaBE4, SaBE4-Gam, Sa(KKH)-BE3, Target-AID, Target-AID-NG, xBE3, eA3A-BE3, BE-PLUS, TAM, CRISPR-X, ABE7.9, ABE7.10, ABE7.10*, xABE, ABESa, ABE8e, VQR-ABE, VRER-ABE, Sa(KKH)-ABE, and CRISPR-SKIP. Additional examples of base editors can be found, for example, in US Publication No. 2018/0312825A1, US Publication No. 2018/0312828A1, and PCT Publication No. WO 2018/165629A1, which are incorporated by reference herein in their entireties.

In some embodiments, the base editor has been further modified to inhibit base excision repair at the target site and induce cellular mismatch repair. Any of the Cas endonucleases described herein may be fused to a Gam domain (bacteriophage Mu protein) to protect the Cas endonuclease from degradation and exonuclease activity. See, e.g., Eid et al. Biochem. J. (2018) 475(11): 1955-1964.

In some embodiments, the Cas endonuclease belongs to class 2 type V of Cas endonuclease. Class 2 type V Cas endonucleases can be further categorized as type V-A, type V-B, type V-C, and type V-U. See, e.g., Stella et al. Nature Structural & Molecular Biology (2017). In some embodiments, the Cas endonuclease is a type V-A Cas endonuclease, such as a Cpf1 nuclease. In some embodiments, the Cas endonuclease is a type V-B Cas endonuclease, such as a C2c1 endonuclease. See, e.g., Shmakov et al. Mol Cell (2015) 60: 385-397. In some embodiments, the Cas endonuclease is Mad7.

Alternatively, or in addition, the Cas endonuclease is a Cpf1 nuclease or a variant thereof. As will be appreciated by one of skill in the art, the Cas endonuclease Cpf1 nuclease may also be referred to as Cas12a. See, e.g., Strohkendl et al. Mol. Cell (2018) 71: 1-9. In some embodiments, the host cell expresses a Cpf1 nuclease derived from Provetella spp. or Francisella spp., Acidaminococcus sp. (AsCpf1), Lachnospiraceae bacterium (LpCpf1), or Eubacterium rectale. In some embodiments, the nucleotide sequence encoding the Cpf1 nuclease may be codon optimized for expression in a host cell. In some embodiments, the nucleotide sequence encoding the Cpf1 endonuclease is further modified to alter the activity of the protein.

A catalytically inactive variant of Cpf1 (Cas12a) may be referred to dCas12a. As described herein, catalytically inactive variants of Cpf1 maybe fused to a function domain to form a base editor. See, e.g., Rees et al. Nature Reviews Genetics (2018) 19:770-788. In some embodiments, the catalytically inactive Cas endonuclease is dCas12a. In some embodiments, the endonuclease comprises a dCas12a fused to one or more uracil glycosylase inhibitor (UGI) domains. In some embodiments, the endonuclease comprises a dCas12a fused to an adenine base editor (ABE), for example an ABE evolved from the RNA adenine deaminase TadA. In some embodiments, the endonuclease comprises a dCas12a fused to a ABE8e. In some embodiments, the endonuclease comprises a dCas12a fused to cytodine deaminase enzyme (e.g., APOBEC deaminase, pmCDA1, activation-induced cytidine deaminase (AID)). In some embodiments, the endonuclease comprised a dCas12a fused to a BE4max.

Alternatively, or in addition, the Cas endonuclease may be a Cas14 endonuclease or variant thereof. In contrast to Cas9 endonucleases, Cas14 endonucleases are derived from archaea and tend to be smaller in size (e.g., 400-700 amino acids). endonucleases do not require a PAM sequence. See, e.g., Harrington et al. Science (2018).

Any of the Cas endonucleases described herein may be modulated to regulate levels of expression and/or activity of the Cas endonuclease at a desired time. For example, it may be advantageous to increase levels of expression and/or activity of the Cas endonuclease during particular phase(s) of the cell cycle. It has been demonstrated that levels of homology-directed repair are reduced during the G1 phase of the cell cycle, therefore increasing levels of expression and/or activity of the Cas endonuclease during the S phase, G2 phase, and/or M phase may increase homology-directed repair following the Cas endonuclease editing. In some embodiments, levels of expression and/or activity of the Cas endonuclease are increased during the S phase, G2 phase, and/or M phase of the cell cycle. In one example, the Cas endonuclease fused to the N-terminal region of human Geminin. See, e.g., Gutschner et al. Cell Rep. (2016) 14(6): 1555-1566. In some embodiments, levels of expression and/or activity of the Cas endonuclease are reduced during the G1 phase. In one example, the Cas endonuclease is modified such that it has reduced activity during the G1 phase. See, e.g., Lomova et al. Stem Cells (2018).

Alternatively, or in addition, any of the Cas endonucleases described herein may be fused to an epigenetic modifier (e.g., a chromatin-modifying enzyme, e.g., DNA methylase, histone deacetylase). See, e.g., Kungulovski et al. Trends Genet. (2016) 32(2):101-113. Cas endonucleases fused to an epigenetic modifier may be referred to as “epieffectors” and may allow for temporal and/or transient endonuclease activity. In some embodiments, the Cas endonuclease is a dCas9 fused to a chromatin-modifying enzyme.

Additionally the compositions and methods described herein can be used in prime editing methods of CRISPR. CRISPR-based prime editor systems can comprise a Cas9 nickase fused to a M-MLV reverse transcriptase (RT). The prime editor system also uses a prime editing guide RNA (pegRNA). Prime editing allows more base substitutions than CRISPR-based base editors and can be used to generate the genetically engineered hematopoietic stem or progenitor cells described herein wherein the nucleotide substitution is within a sequence encoding a splice element, wherein the nucleotide substitution results in an alternative splicing of a transcript encoded by the gene.

In further embodiments, homology directed repair is used to generate the genetically engineered hematopoietic stem or progenitor cells described herein wherein the nucleotide substitution is within a sequence encoding a splice element, wherein the nucleotide substitution results in an alternative splicing of a transcript encoded by the gene.

In some embodiments, the present disclosure provides compositions and methods for inhibiting a lineage-specific cell-surface antigen in hematopoietic cells using a CRISPR-based base editor system, wherein guide RNA sequence hybridizes to the nucleotide sequence encoding the lineage-specific cell-surface antigen. In some embodiments, the present disclosure provides compositions and methods for inhibiting more than one lineage-specific cell-surface antigen in hematopoietic cells using a CRISPR-based base editor system, wherein guide RNA sequence hybridizes to the nucleotide sequence encoding the lineage-specific cell-surface antigen.

In some embodiments, the present disclosure provides compositions and methods for altering a lineage-specific cell-surface antigen in hematopoietic cells using a CRISPR-based base editor system, wherein guide RNA sequence hybridizes to the nucleotide sequence encoding the lineage-specific cell-surface antigen. In some embodiments, the present disclosure provides compositions and methods for altering more than one lineage-specific cell-surface antigen in hematopoietic cells using a CRISPR-based base editor system, wherein guide RNA sequence hybridizes to the nucleotide sequence encoding the lineage-specific cell-surface antigen.

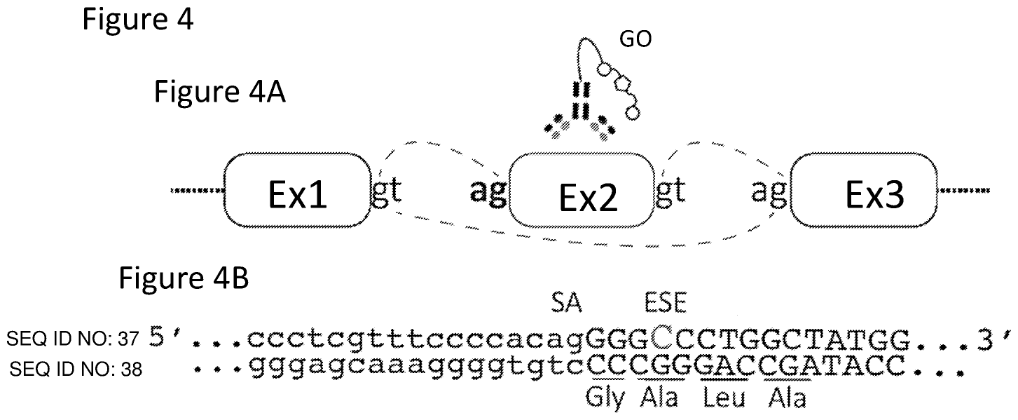

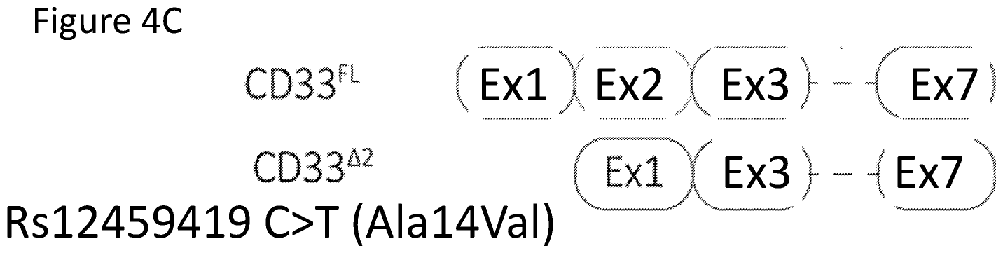

In some embodiments, the lineage-specific cell-surface antigen is CD33 and the gRNA hybridizes to a portion of the nucleotide sequence that encodes the CD33. In some embodiments, the gRNA hybridizes sequences flanking exon 2 of CD33 (FIG. 4). Examples of gRNAs that target CD33 are provided in Table 4, although additional gRNAs may be developed that hybridize to the pertinent nucleotide sequences of CD33 and can be used in the methods described herein.

In some instances, the gRNA for use in the present disclosure may comprise a spacer sequence at least 90% (e.g., at least 93%, 95%, 96%, 97%, 98%, or 99%) identical to any of the exemplary guide RNA sequences in Table 4.

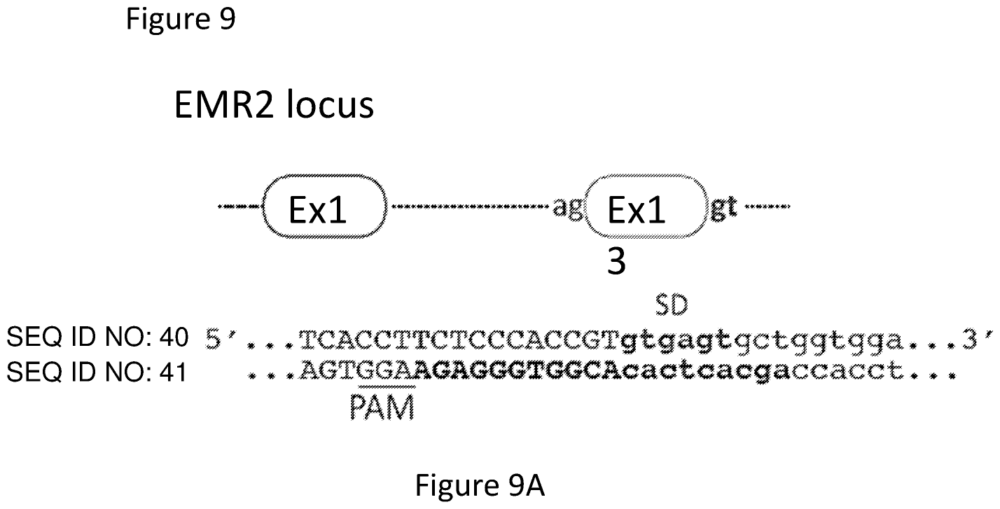

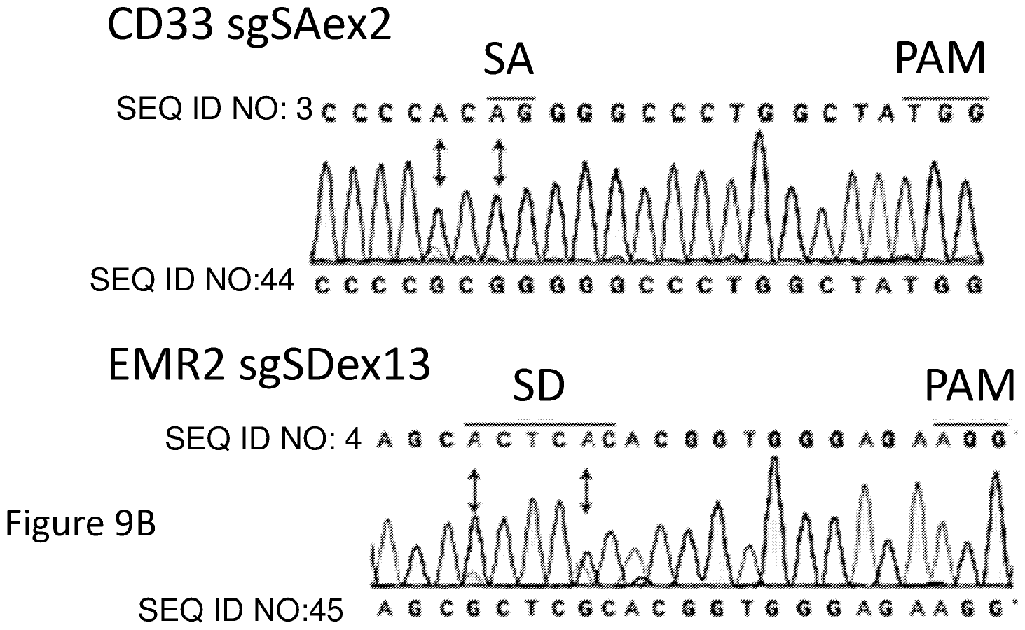

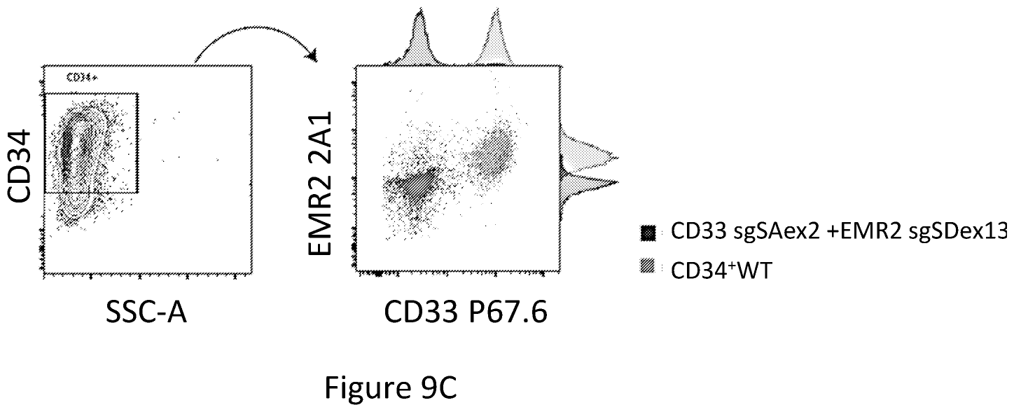

In some embodiments, the lineage-specific cell-surface antigen is EMR2 and the gRNA hybridizes to a portion of the nucleotide sequence that encodes the EMR2. In some embodiments, the gRNA hybridizes sequences flanking exon 13 of the nucleotide sequence encoding EMR2 (FIG. 9B). Examples of gRNAs that target EMR2 are provided in below, although additional gRNAs may be developed that hybridize to the pertinent nucleotide sequences of EMR2 and can be used in the methods described herein.

In some instances, the gRNA for use in the present disclosure may comprise a spacer sequence at least 90% (e.g., at least 93%, 95%, 96%, 97%, 98%, or 99%) identical to any of SEQ ID NO: 4 and 46-47.

TABLE 4
Example Guide RNAs for
Base Editing CD33 and EMR2

| Lineage- | | | | |
|---|---|---|---|---|
| Specific | Base | Splice | | |
| Antigen | Editor | Element | gRNA | |
| CD33 | BE4max | ESE | Sg207 | SEQ ID |
| | | | GGGCcccTGT | NO: 1 |
| | | | GGGGAAACGA | |
| | | | GGG | |
| CD33 | BE4max | SA | Sg208 | SEQ ID |
| | | | AGGGCcccTG | NO: 2 |
| | | | TGGGGAAACG | |
| | | | AGG | |
| CD33 | ABE8e | SA | sgSA | SEQ ID |
| | | | CCCCaCaGGG | NO: 3 |
| | | | GCCCTGGCTA | |
| | | | TGG | |
| EMR2 | ABE | SD | sgRNA-EMR- | SEQ ID |
| | | | SDex13 | NO: 4 |
| | | | AGCACTCACA | |
| | | | CGGTGGGAGA | |
| | | | AGG | |
| EMR2 | ABE | | sgRNA-EMR2- | SEQ ID |
| | | | SDex18 | NO: 46 |
| | | | ATCTTACCTT | |
| | | | GTGTTCCGGA | |
| | | | GGG | |
| EMR2 | ABE | | sgRNA-EMR2- | SEQ ID |
| | | | SDex19 | NO: 47 |
| | | | GTGGTACCTG | |
| | | | CTGGCTGAGG | |
| | | | AGG | |

Additionally, the present disclosure provides compositions and methods for the combined inhibition of a first lineage-specific cell-surface antigen and at least one additional lineage-specific cell-surface antigen, i.e., a first lineage specific antigen, a second lineage specific antigen, a third lineage specific antigen, a fourth lineage specific antigen, etc.

Additionally, the present disclosure provides compositions and methods for the combined alteration of a first lineage-specific cell-surface antigen and at least one additional lineage-specific cell-surface antigen, i.e., a first lineage specific antigen, a second lineage specific antigen, a third lineage specific antigen, a fourth lineage specific antigen, etc.

In some embodiments, the first lineage specific antigen is CD33. In some embodiments, the second lineage specific antigen is EMR2.

The “percent identity” of two nucleic acids is determined using the algorithm of Karlin and Altschul Proc. Natl. Acad. Sci. USA (1990) 87:2264-68, modified as in Karlin and Altschul Proc. Natl. Acad. Sci. USA (1993) 90:5873-77. Such an algorithm is incorporated into the NBLAST and XBLAST programs (version 2.0) of Altschul, et al. J. Mol. Biol. (1990) 215:403-10. BLAST nucleotide searches can be performed with the NBLAST program, score=100, wordlength-12 to obtain nucleotide sequences homologous to the nucleic acid molecules of the invention. Where gaps exist between two sequences, Gapped BLAST can be utilized as described in Altschul et al., Nucleic Acids Res. (1997) 25(17):3389-3402. When utilizing BLAST and Gapped BLAST programs, the default parameters of the respective programs (e.g., XBLAST and NBLAST) can be used.

Also provided herein are methods of producing a cell that is deficient or altered in a lineage-specific cell-surface antigen involving providing a cell and introducing into the cell components of a CRISPR-based base editor system for genome editing. In some embodiments, a nucleic acid that comprises a guide RNA (gRNA) that hybridizes or is predicted to hybridize to a portion of the nucleotide sequence that encodes the lineage-specific cell-surface antigen is introduced into the cell. In some embodiments, the gRNA is introduced into the cell on a vector. In some embodiments, a CRISPR-based base editor is introduced into the cell. In some embodiments, the CRISPR-based base editor is introduced into the cell as a nucleic acid encoding a CRISPR-based base editor. In some embodiments, the gRNA and a nucleotide sequence encoding a CRISPR-based base editor are introduced into the cell on the same nucleic acid (e.g., the same vector). In some embodiments, the CRISPR-based base editor is introduced into the cell in the form of a protein. In some embodiments, the CRISPR-based base editor and the gRNA are pre-formed in vitro and are introduced to the cell in as a complex. In some embodiments, the complex (e.g., ribonucleoprotein complex) is introduced using electroporation.

Also provided herein are methods of producing a cell that is deficient or altered in more than one lineage-specific cell-surface antigen involving providing a cell and introducing into the cell components of more than one CRISPR-based base editor system for genome editing, i.e., a CRISPR-based editor system for genome editing a lineage-specific cell-surface antigen, and a CRISPR-based editor system for genome editing an at least one additional lineage-specific cell-surface antigen, e.g., a first and a second CRISPR-based base editor system. In some embodiments, components of more than one CRISPR-based base editor system is introduced into the cell. In some embodiments, a nucleic acid that comprises a guide RNA (gRNA) that hybridizes or is predicted to hybridize to a portion of the nucleotide sequence that encodes at least one additional lineage-specific cell-surface antigen is introduced into the cell. In some embodiments, the gRNA is introduced into the cell on a vector. In some embodiments, a CRISPR-based base editor is introduced into the cell. In some embodiments, the CRISPR-based base editor is introduced into the cell as a nucleic acid encoding a CRISPR-based base editor. In some embodiments, the gRNA and a nucleotide sequence encoding a CRISPR-based base editor are introduced into the cell on the same nucleic acid (e.g., the same vector). In some embodiments, the CRISPR-based base editor is introduced into the cell in the form of a protein. In some embodiments, the CRISPR-based base editor and the gRNA are pre-formed in vitro and are introduced to the cell in as a complex. In some embodiments, the complex (e.g., ribonucleoprotein complex) is introduced using electroporation.

In some embodiments, the first CRISPR-based base editor system is introduced into the cell by a different method than the subsequent based base editor system. In some embodiments, all of the CRISPR-based base editor systems are introduced into the cell using the same method.

The present disclosure further provides engineered, non-naturally occurring vectors and vector systems, which can encode one or more components of a CRISPR-based base editor system, wherein the vector comprises a polynucleotide encoding (i) a (CRISPR)-Cas system guide RNA that hybridizes to the lineage specific antigen sequence and (ii) a CRISPR-based base editor.

Vectors of the present disclosure can drive the expression of one or more sequences in mammalian cells using a mammalian expression vector. Examples of mammalian expression vectors include pCDM8 (Seed, Nature (1987) 329: 840) and pMT2PC (Kaufman, et al., EMBO J. (1987) 6: 187). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd eds., Cold Spring Harbor Laboratory, Cold Spring Harbor Laboratory Press, Cold Spring Harbor, N.Y., 1989.

The vectors of the present disclosure are capable of directing expression of the nucleic acid preferentially in a particular cell type (e.g., tissue-specific regulatory elements are used to express the nucleic acid). Such regulatory elements include promoters that may be tissue specific or cell specific. The term “tissue specific” as it applies to a promoter refers to a promoter that is capable of directing selective expression of a nucleotide sequence of interest to a specific type of tissue (e.g., seeds) in the relative absence of expression of the same nucleotide sequence of interest in a different type of tissue. The term “cell type specific” as applied to a promoter refers to a promoter that is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining.

Conventional viral and non-viral based gene transfer methods can be used to introduce nucleic acids encoding CRISPR-based base editors in mammalian cells or target tissues. Such methods can be used to administer nucleic acids encoding components of a CRISPR-based base editor system to cells in culture, or in a host organism.

Non-viral vector delivery systems include DNA plasmids, RNA (e.g., a transcript of a vector described herein), naked nucleic acid, and nucleic acid complexed with a delivery vehicle. In one embodiment, the non-viral vector delivery system used is a pre-formed ribonucleoprotein complex (e.g., a complex comprising a CRISPR-based base editor protein in complex with the targeting gRNA). The pre-formed ribonucleoprotein complex may then be introduced into the cell via electroporation, biolistic bombardment, or other physical methods of delivery. In one embodiment, electroporation is used to introduce the pre-formed ribonucleoprotein complex into the cell. See, e.g., Example 1.

Viral vector delivery systems include DNA and RNA viruses, which have either episomal or integrated genomes after delivery to the cell. Viral vectors can be administered directly to patients (in vivo) or they can be used to manipulate cells in vitro or ex vivo, where the modified cells may be administered to patients. In one embodiment, the present disclosure utilizes viral based systems including, but not limited to retroviral, lentivirus, adenoviral, adeno-associated and herpes simplex virus vectors for gene transfer. Furthermore, the present disclosure provides vectors capable of integration in the host genome, such as retrovirus or lentivirus. Preferably, the vector used for the expression of a CRISPR-based base editor system of the present disclosure is a lentiviral vector.

In one embodiment, the disclosure provides for introducing one or more vectors encoding CRISPR-based base editor into eukaryotic cell. The cell can be a cancer cell. Alternatively, the cell is a hematopoietic cell, such as a hematopoietic stem cell. Examples of stem cells include pluripotent, multipotent and unipotent stem cells. Examples of pluripotent stem cells include embryonic stem cells, embryonic germ cells, embryonic carcinoma cells and induced pluripotent stem cells (iPSCs). In a preferred embodiment, the disclosure provides introducing CRISPR-based base editor into a hematopoietic stem cell.

The vectors of the present disclosure are delivered to the eukaryotic cell in a subject. Modification of the eukaryotic cells via CRISPR-based base editor system can take place in a cell culture, where the method comprises isolating the eukaryotic cell from a subject prior to the modification. In some embodiments, the method further comprises returning said eukaryotic cell and/or cells derived therefrom to the subject.

### Combined Therapy

As described herein, agents comprising an antigen-binding fragment that binds to a lineage-specific cell-surface antigen (e.g., CD33) may be administered to a subject in combination with hematopoietic cells that are deficient for the lineage-specific cell-surface antigen or an epitope thereof, e.g., hematopoietic stem or progenitor cells produced using a CRISPR-based base editor system and a gRNA described herein, e.g., wherein the gRNA comprises the nucleotide sequence of: SEQ ID NO: 1; SEQ ID NO: 2; or SEQ ID NO: 3.

As described herein, agents comprising an antigen-binding fragment that binds to a lineage-specific cell-surface antigen (e.g., EMR2) may be administered to a subject in combination with hematopoietic cells that are deficient for the lineage-specific cell-surface antigen or an epitope thereof, e.g., hematopoietic stem or progenitor cells produced using a CRISPR-based base editor system and gRNA described herein, e.g., wherein the gRNA comprises the nucleotide sequence of SEQ ID NO: 4; SEQ ID NO: 46; and SEQ ID NO: 47.

Also as described herein, agents comprising an antigen-binding fragment that binds to a lineage-specific cell-surface antigen (e.g., CD33) and agents comprising an antigen-binding fragment that binds to at least one additional lineage-specific cell-surface antigen may be administered to a subject in combination with hematopoietic cells that are deficient for the lineage-specific cell-surface antigen or an epitope thereof, e.g., hematopoietic stem or progenitor cells produced using a CRISPR-based base editor system and a gRNA described herein, e.g., wherein the gRNA comprises the nucleotide sequence of:
(SEQ ID NO: 1);
(SEQ ID NO: 2); or
(SEQ ID NO: 3); and
(SEQ ID NO: 4);
(SEQ ID NO: 46); or
(SEQ ID NO: 47).

As used herein, “subject,” “individual,” and “patient” are used interchangeably, and refer to a vertebrate, preferably a mammal such as a human. Mammals include, but are not limited to, human primates, non-human primates or murine, bovine, equine, canine or feline species. In some embodiments, the subject is a human patient having a hematopoietic malignancy.

In some embodiments, the agents and/or the hematopoietic cells may be mixed with a pharmaceutically acceptable carrier to form a pharmaceutical composition, which is also within the scope of the present disclosure.

To perform the methods described herein, an effective amount of the agent(s) comprising an antigen-binding fragment that binds to a lineage-specific cell-surface antigen(s) and an effective amount of hematopoietic cells can be co-administered to a subject in need of the treatment. As used herein the term “effective amount” may be used interchangeably with the term “therapeutically effective amount” and refers to that quantity of an agent, cell population, or pharmaceutical composition (e.g., a composition comprising agents and/or hematopoietic cells) that is sufficient to result in a desired activity upon administration to a subject in need thereof. Within the context of the present disclosure, the term “effective amount” refers to that quantity of a compound, cell population, or pharmaceutical composition that is sufficient to delay the manifestation, arrest the progression, relieve or alleviate at least one symptom of a disorder treated by the methods of the present disclosure. Note that when a combination of active ingredients is administered the effective amount of the combination may or may not include amounts of each ingredient that would have been effective if administered individually.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human. In some embodiments, the subject is a human patient having a hematopoietic malignancy.

As described herein, the hematopoietic cells and/or immune cells expressing chimeric receptors may be autologous to the subject, i.e., the cells are obtained from the subject in need of the treatment, genetically engineered to be deficient or altered for expression of the cell-surface lineage-specific antigen or for expression of the chimeric receptor constructs, and then administered to the same subject. Administration of autologous cells to a subject may result in reduced rejection of the host cells as compared to administration of non-autologous cells. Alternatively, the host cells are allogeneic cells, i.e., the cells are obtained from a first subject, genetically engineered to be deficient or altered for expression of the cell-surface lineage-specific antigen or for expression of the chimeric receptor construct and administered to a second subject that is different from the first subject but of the same species. For example, allogeneic immune cells may be derived from a human donor and administered to a human recipient who is different from the donor.

In some embodiments, the immune cells expressing any of the chimeric receptors described herein are administered to a subject in an amount effective in to reduce the number of target cells (e.g., cancer cells) by least 20%, e.g., 50%, 80%, 100%, 2-fold, 5-fold, 10-fold, 20-fold, 50-fold, 100-fold or more.

A typical amount of cells, i.e., immune cells or hematopoietic cells, administered to a mammal (e.g., a human) can be, for example, in the range of one million to 100 billion cells; however, amounts below or above this exemplary range are also within the scope of the present disclosure. For example, the daily dose of cells can be about 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), preferably about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), more preferably about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells, or a range defined by any two of the foregoing values).

In one embodiment, the chimeric receptor (e.g., a nucleic acid encoding the chimeric receptor) is introduced into an immune cell, and the subject (e.g., human patient) receives an initial administration or dose of the immune cells expressing the chimeric receptor. One or more subsequent administrations of the agent (e.g., immune cells expressing the chimeric receptor) may be provided to the patient at intervals of 15 days, 14, 13, 12, 11, 10, 9, 8, 7, 6, 5, 4, 3, or 2 days after the previous administration. More than one dose of the agent can be administered to the subject per week, e.g., 2, 3, 4, or more administrations of the agent. The subject may receive more than one doses of the agent (e.g., an immune cell expressing a chimeric receptor) per week, followed by a week of no administration of the agent, and finally followed by one or more additional doses of the agent (e.g., more than one administration of immune cells expressing a chimeric receptor per week). The immune cells expressing a chimeric receptor may be administered every other day for 3 administrations per week for two, three, four, five, six, seven, eight or more weeks.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (i.e., the period prior to clinical manifestation of a disease) and/or reduce the risk of developing or worsening a disease. For example, in connection with cancer the term “treat” may mean eliminate or reduce a patient's tumor burden, or prevent, delay or inhibit metastasis.

In some embodiments, an agent(s) comprising an antigen-binding fragment that binds a lineage-specific cell-surface antigen(s) and a population of hematopoietic cells deficient or altered in the lineage-specific cell-surface antigen(s). Accordingly, in such therapeutic methods, the agent(s) recognizes (binds) a target cell expressing the lineage-specific cell-surface antigen(s) repopulation of a cell type that is targeted by the agent(s). In some embodiments, the treatment of the patient can involve the following steps: (1) administering a therapeutically effective amount of an agent(s) targeting a lineage-specific cell-surface antigen(s) to the patient; and (2) infusing or reinfusing the patient with hematopoietic stem cells, either autologous or allogenic, where the hematopoietic cells have reduced or altered expression of a lineage specific disease-associated antigen(s). In some embodiments, the treatment of the patient can involve the following steps: (1) administering a therapeutically effective amount of an immune cell expressing a chimeric receptor to the patient, wherein the immune cell comprises a nucleic acid sequence encoding a chimeric receptor that binds a lineage-specific cell-surface, disease-associated antigen(s); and (2) infusing or reinfusing the patient with hematopoietic cells (e.g., hematopoietic stem cells), either autologous or allogenic, where the hematopoietic cells have reduced or altered expression of a lineage specific disease-associated antigen(s).

The efficacy of the therapeutic methods using an agent(s) comprising an antigen-binding fragment that binds a cell-surface lineage-specific antigen(s) and a population of hematopoietic cells deficient or altered in the lineage-specific cell-surface antigen(s) may be assessed by any method known in the art and would be evident to a skilled medical professional. For example, the efficacy of the therapy may be assessed by survival of the subject or cancer burden in the subject or tissue or sample thereof. In some embodiments, the efficacy of the therapy is assessed by quantifying the number of cells belonging to a particular population or lineage of cells. In some embodiments, the efficacy of the therapy is assessed by quantifying the number of cells presenting the cell-surface lineage-specific antigen.

In some embodiments, the agent comprising an antigen-binding fragment that binds to the cell-surface lineage-specific antigen and the population of hematopoietic cells is administered concomitantly.

In some embodiments, the agent(s) comprising an antigen-binding fragment that binds a lineage-specific cell-surface antigen(s) (e.g., immune cells expressing a chimeric receptor as described herein) is administered prior to administration of the hematopoietic cells. In some embodiments, the agent(s) comprising an antigen-binding fragment that binds a lineage-specific cell-surface antigen(s) (e.g., immune cells expressing a chimeric receptor as described herein) is administered at least about 1 day, 2 days, 3 days, 4 days, 5 days, 6 days, 7 days, 8 days, 9 days, 10 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the hematopoietic cells. In some embodiments, the agent(s) comprising an antigen-binding fragment that binds a lineage-specific cell-surface antigen(s) (e.g., immune cells expressing a chimeric receptor as described herein) is administered multiple times to the subject prior to administration of the hematopoietic cells.

In some embodiments, the hematopoietic cells are administered prior to the agent(s) comprising an antigen-binding fragment that binds a lineage-specific cell-surface antigen(s) (e.g., immune cells expressing a chimeric receptor as described herein). In some embodiments, the population of hematopoietic cells is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of the agent(s) comprising an antigen-binding fragment that binds to the lineage-specific cell-surface antigen(s).

In some embodiments, the agent(s) targeting the lineage-specific cell-surface antigen and the population of hematopoietic cells are administered at substantially the same time. In some embodiments, agent(s) targeting the lineage-specific cell-surface antigen(s) are administered and the patient is assessed for a period of time, after which the population of hematopoietic cells is administered. In some embodiments, the population of hematopoietic cells is administered and the patient is assessed for a period of time, after which agent(s) targeting the lineage-specific cell-surface antigen(s) is administered.

Also within the scope of the present disclosure are multiple administrations (e.g., doses) of the agents and/or populations of hematopoietic cells. In some embodiments, the agents and/or populations of hematopoietic cells are administered to the subject once. In some embodiments, agents and/or populations of hematopoietic cells are administered to the subject more than once (e.g., at least 2, 3, 4, 5, or more times). In some embodiments, the agents and/or populations of hematopoietic cells are administered to the subject at a regular interval, e.g., every six months.

In some embodiments, the subject is a human subject having a hematopoietic malignancy. As used herein a hematopoietic malignancy refers to a malignant abnormality involving hematopoietic cells (e.g., blood cells, including progenitor and stem cells). Examples of hematopoietic malignancies include, without limitation, Hodgkin's lymphoma, non-Hodgkin's lymphoma, leukemia, or multiple myeloma. Leukemias include acute myeloid leukemia, acute lymphoid leukemia, chronic myelogenous leukemia, acute lymphoblastic leukemia or chronic lymphoblastic leukemia, and chronic lymphoid leukemia.

In some embodiments, the leukemia is acute myeloid leukemia (AML). AML is characterized as a heterogeneous, clonal, neoplastic disease that originates from transformed cells that have progressively acquired critical genetic changes that disrupt key differentiation and growth-regulatory pathways. (Dohner et al., NEJM, (2015) 373:1136). CD33 glycoprotein is expressed on the majority of myeloid leukemia cells as well as on normal myeloid and monocytic precursors and has been considered to be an attractive target for AML therapy (Laszlo et al., Blood Rev. (2014) 28(4):143-53). While clinical trials using anti CD33 monoclonal antibody-based therapy have shown improved survival in a subset of AML patients when combined with standard chemotherapy, these effects were also accompanied by safety and efficacy concerns.

Other efforts aimed at targeting AML cells have involved the generation of T cells expressing chimeric antigen receptors (CARs) that selectively target CD33 in AML. Buckley et al., Curr. Hematol. Malig. Rep. (2015) 2:65. However, the data is limited and there are uncertainties about how effective (whether all targeted cells are eliminated) this approach may be in treating the patient. Additionally, since myeloid lineage cells are indispensable for life, depleting a subject of myeloid lineage cells could have detrimental effects on survival of the patient. The present disclosure aims at, at least in part, solving such problems associated with AML treatment.

Alternatively or in addition, the methods described herein may be used to treat non-hematopoietic cancers, including without limitation: lung cancer; ear, nose and throat cancer; colon cancer; melanoma; pancreatic cancer; prostate cancer; breast cancer; ovarian cancer; basal cell carcinoma; biliary tract cancer; bladder cancer; bone cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; liver cancer; fibroma, neuroblastoma; oral cavity cancer (e.g., lip, tongue, mouth, and pharynx); retinoblastoma; rhabdomyosarcoma; renal cancer; cancer of the respiratory system; sarcoma; skin cancer; stomach cancer; testicular cancer; thyroid cancer; uterine cancer; cancer of the urinary system, as well as other carcinomas and sarcomas.

Carcinomas are cancers of epithelial origin. Carcinomas intended for treatment with the methods of the present disclosure include, but are not limited to, acinar carcinoma, acinous carcinoma, alveolar adenocarcinoma (also called adenocystic carcinoma, adenomyoepithelioina, cribriform carcinoma and cylindroma), carcinoma adenomatosum, adenocarcinoma, carcinoma of adrenal cortex, alveolar carcinoma, alveolar cell carcinoma (also called bronchiolar carcinoma, alveolar cell tumor and pulmonary adenomatosis), basal cell carcinoma, carcinoma basocellulare (also called basaloma, or basiloma, and hair matrix carcinoma), basaloid carcinoma, basosquamous cell carcinoma, breast carcinoma, bronchioalveolar carcinoma, bronchiolar carcinoma, bronchogenic carcinoma, cerebriform carcinoma, cholangiocellular carcinoma (also called cholangioma and cholangiocarcinoma), chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epibulbar carcinoma, epidermoid carcinoma, carcinoma epitheliale adenoides, carcinoma exulcere, carcinoma fibrosum, gelatiniform carcinoma, gelatinous carcinoma, giant cell carcinoma, gigantocellulare, glandular carcinoma, granulosa cell carcinoma, hair-matrix carcinoma, hematoid carcinoma, hepatocellular carcinoma (also called hepatoma, malignant hepatoma and hepatocarcinoma), Huirthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, lenticular carcinoma, carcinoma lenticulare, lipomatous carcinoma, lymphoepithelial carcinoma, carcinoma mastitoides, carcinoma medullare, medullary carcinoma, carcinoma melanodes, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, carcinoma nigrum, oat cell carcinoma, carcinoma ossificans, osteoid carcinoma, ovarian carcinoma, papillary carcinoma, periportal carcinoma, preinvasive carcinoma, prostate carcinoma, renal cell carcinoma of kidney (also called adenocarcinoma of kidney and hypemephoroid carcinoma), reserve cell carcinoma, carcinoma sarcomatodes, scheinderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, carcinoma vilosum. In preferred embodiments, the methods of the present disclosure are used to treat subjects having cancer of the breast, cervix, ovary, prostate, lung, colon and rectum, pancreas, stomach or kidney.

Sarcomas are mesenchymal neoplasms that arise in bone and soft tissues. Different types of sarcomas are recognized and these include: liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., non-bone) Ewing's sarcoma, and primitive neuroectodermal tumor [PNET]), synovial sarcoma, angiosarcomas, hemangiosarcomas, lymphangiosarcomas, Kaposi's sarcoma, hemangioendothelioma, fibrosarcoma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), osteosarcoma (also known as osteogenic sarcoma)-skeletal and extraskeletal, and chondrosarcoma.

In some embodiments, the cancer to be treated can be a refractory cancer. A “refractory cancer,” as used herein, is a cancer that is resistant to the standard of care prescribed. These cancers may appear initially responsive to a treatment (and then recur), or they may be completely non-responsive to the treatment. The ordinary standard of care will vary depending upon the cancer type, and the degree of progression in the subject. It may be a chemotherapy, or surgery, or radiation, or a combination thereof. Those of ordinary skill in the art are aware of such standards of care. Subjects being treated according to the present disclosure for a refractory cancer therefore may have already been exposed to another treatment for their cancer. Alternatively, if the cancer is likely to be refractory (e.g., given an analysis of the cancer cells or history of the subject), then the subject may not have already been exposed to another treatment. Examples of refractory cancers include, but are not limited to, leukemia, melanomas, renal cell carcinomas, colon cancer, liver (hepatic) cancers, pancreatic cancer, Non-Hodgkin's lymphoma and lung cancer.

Any of the immune cells expressing chimeric receptors described herein may be administered in a pharmaceutically acceptable carrier or excipient as a pharmaceutical composition.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g. Remington: The Science and Practice of Pharmacy 20th Ed. (2000) Lippincott Williams and Wilkins, Ed. K. E. Hoover.

### Kits for Therapeutic Uses

Also within the scope of the present disclosure are kits for use of the agents targeting lineage-specific cell-surface antigens in combination with populations of hematopoietic cells that are deficient in the cell-surface lineage-specific antigen(s). Such kits may include one or more containers comprising a first pharmaceutical composition that comprises any agent(s) comprising an antigen-binding fragment that binds a lineage-specific cell-surface antigen(s) (e.g., immune cells expressing chimeric receptors described herein), and a pharmaceutically acceptable carrier, and a second pharmaceutical composition that comprises a population of hematopoietic cells that are deficient in one or more cell-surface lineage-specific antigen(s) (e.g., a hematopoietic stem cell) and a pharmaceutically acceptable carrier.

In some embodiments, a kit described herein comprises a gRNA having a sequence of SEQ ID NOs: 1-3. In some embodiments, a kit described herein comprises a gRNA having a sequence of SEQ ID NOs: 4, and 46-47. In further embodiments, the kit can further comprise a gRNA having a sequence of any of SEQ ID NOs: 1-4 and 46-47. In some embodiments, the kit can further comprise reagents of a CRISPR-based base editor systems, including a catalytically impaired Cas protein fused to a DNA-modifying enzyme, i.e., Cas9 nickase fused to a cytosine or adenosine deaminase (base editor).

In some embodiments, the kit can comprise instructions for use in any of the methods described herein. The included instructions can comprise a description of administration of the first and second pharmaceutical compositions to a subject to achieve the intended activity in a subject. The kit may further comprise a description of selecting a subject suitable for treatment based on identifying whether the subject is in need of the treatment. In some embodiments, the instructions comprise a description of administering the first and second pharmaceutical compositions to a subject who is in need of the treatment.

The instructions relating to the use of the agents targeting cell-surface lineage-specific antigens and the first and second pharmaceutical compositions described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a chimeric receptor variants as described herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (lRL Press, (1986»; and B. Perbal, A practical Guide To Molecular Cloning (1984); F. M. Ausubel et al. (eds.).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: Efficient Editing of HSC/HSPCs with Base Editors

### Materials and Methods

Human Cord Blood CD34+ stem cells were maintained in StemSpan SFEM II (STEMCELL Technologies Inc) containing 1% Penicillin Streptomycin, and the following human cytokines 100 ng/mL TPO, 100 ng/mL SCF, 100 ng/mL IL6 and 100 ng/mL FLT3L and UM171 0.35 nM (Xcessbio, San Diego, CA, USA). All human cytokines were purchased from Biolegend (San Diego, CA, USA).

ABE protein and sgRNA were mixed in P3 buffer (Lonza, Amaxa P3 Primary Cell 4D-Nucleofector Kit) and incubated for 10 mins. The cells were then washed with PBS, resuspended in P3 buffer, mixed with the Cas9/sgRNA RNP complex and then electroporated with the 4D-Nucleofector. After electroporation, cells were cultured at 37° C. until analysis or injection.

Editing HSCs/HSPCs with ABE8e Protein

1 million HSC/HSPCs were nucleofected with 11 ugr ABE8e protein+1.5ugr sgRNA (chemically modified (SEQ ID NO: 3)) using Lonza 4D nucleofector and program DZ100.

Editing HSCs/HSPCs with BE4max Protein

500 000 HSC/HSPCs were nucleofected with 8ugr BE4max protein+1.5ugr sgRNA (chemically modified (SEQ ID NOs: 1 or 2)) using Lonza 4D nucleofector and program DZ100.

After electroporation, cells were cultured at 37° C. until analysis.

### Editing Outcome and Off-Target Assessment:

HTS analysis by CRISPResso2

Primers surrounding a 350 bp region around the protospacer sgRNA 208 or sgRNA SA were designed. Each primer was appended with the appropriate ILLUMINA adapter. The generated PCR products were then reamplified using ILLUMINA forward and reverse index primers to generate a 250-300 bp single-end Illumina sequencing. Then CRISPResso2 was used to align each read to the reference amplicon and identify indels or base changes relative to that reference in a window surrounding the protospacer. Those are quantified by software. See Clement et al. Nature Biotechnology (2019) 37:224-26; Huang et al. Nature Protocols (2021) 16(2): 1089-1128.

PCR

cDNA was prepared from 100 ng of total RNA from the cells using RNA to cDNA EcoDry mix (Takara). Thirty cycles of PCR were performed using primers specific for CD33^{Δ2} (spanning exon junction 1-3), CD33FL (spanning exon junction 2-3 or exon 2), or common to all isoforms (in exons 1, 5 and 7). PCR products were separated by polyacrylamide gel electrophoresis and analyzed by Sanger sequencing.

Flow Cytometry

Human CD34⁺ stem cells were analyzed 7 days after electroporation using the following anti-CD33 antibodies clones, M53 and P67.6, which recognize an epitope located in exon 2.

### Statistics

All statistics were performed using Graphpad Prism 9.1.1. For continuous variables, an unpaired two tailed t-test was performed. Differences between means were considered significant when the p value is <0.05, else not significant (ns; p>0.05).

### Results

Three sgRNAs were designed to induce exon 2 skipping using a BE or ABE base editor (FIG. 4). CD33 mRNA full length (CD33^{FL}) contains 7 exons and exon 2 encodes an Ig-like V-type domain. CD33^{Δ2} lacks exon 2 due to a common polymorphism (rs12459419, A14VSNP)) which changes C>T resulting in an altered exonic splicing enhancer (ESE) site.

mRNA and ribonucleoprotein (RNP) based delivery systems were tested to check the efficiency in primary cells. CD34 cells were electroporated with each of these gRNAs and a base editor (BE) or an adenine base editor (ABE) protein. Specifically, CD34 cells were electroporated with ABE8e and the sgRNA with SEQ ID NO: 3 or BE4max and the sgRNA with SEQ ID NO: 1, or BE4max and the sgRNA with SEQ ID NO: 2 (Table 4).

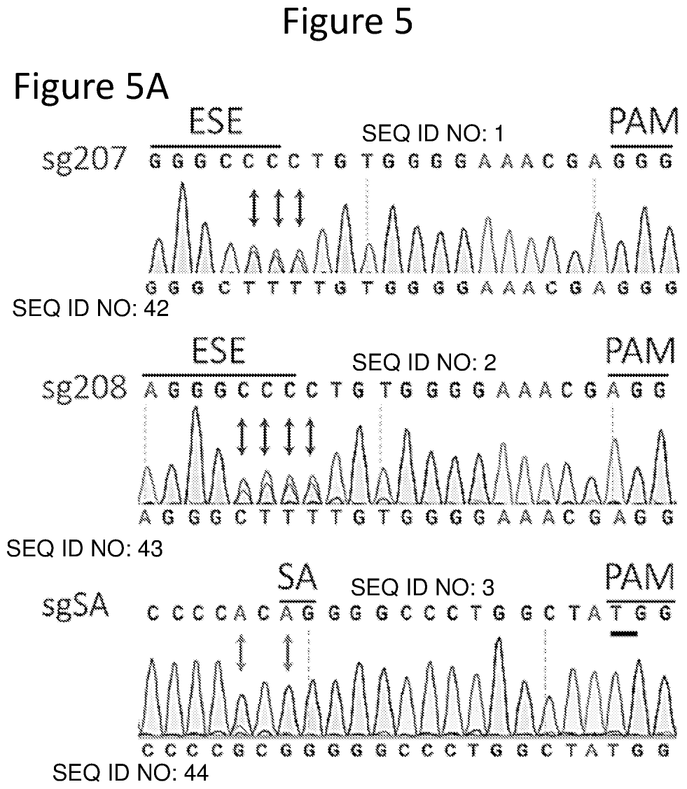

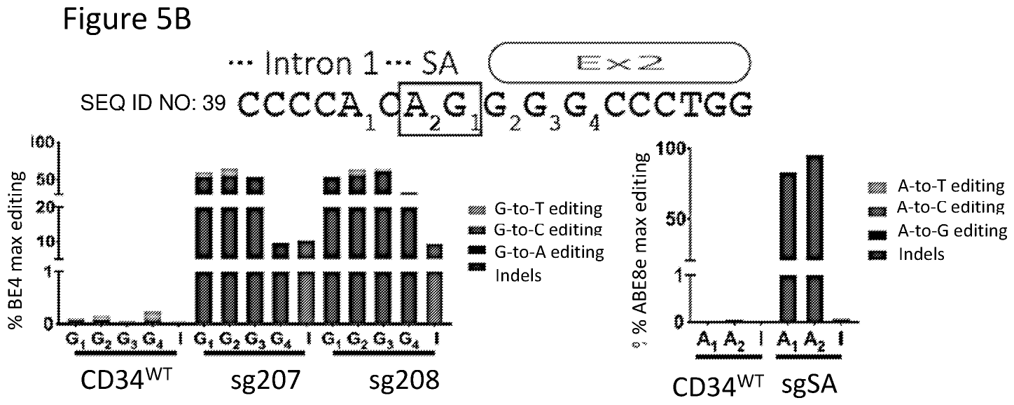

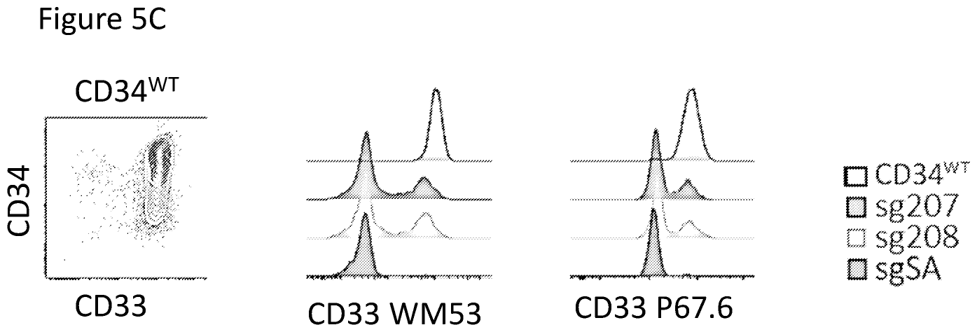

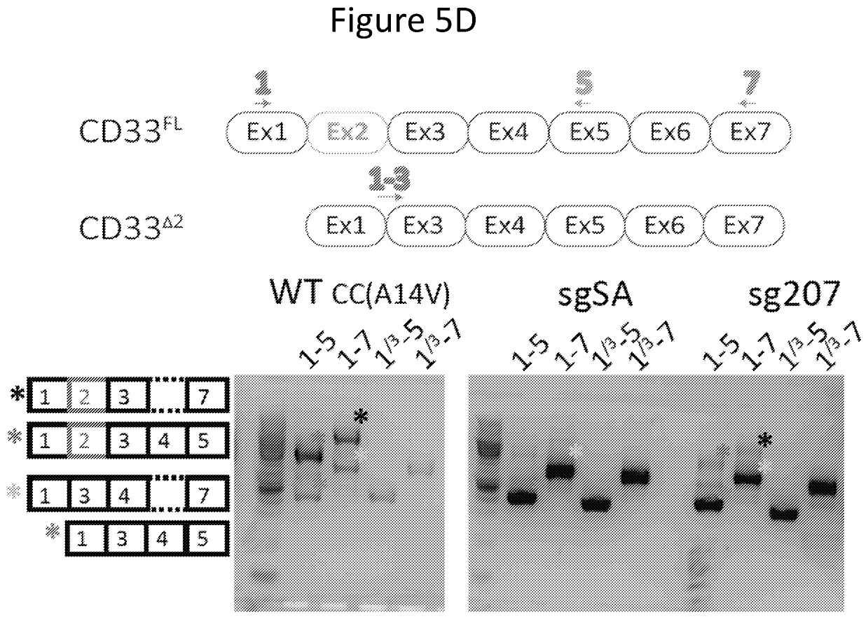

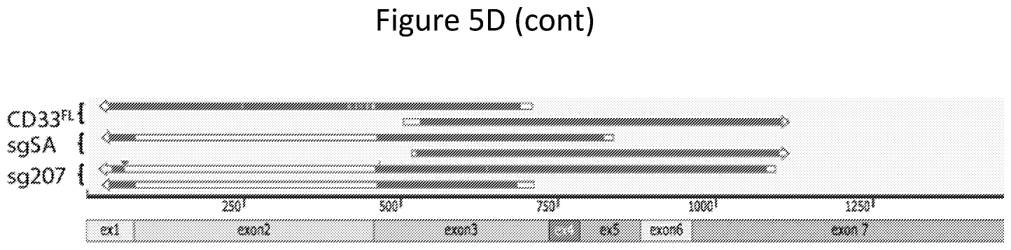

As shown by Sanger sequencing, the BE and ABE introduced either C>T or A>G conversions at the targeted nucleotides (FIG. 5A).

The edited cells were also analyzed using PCR and Illumina MiSeq. These results showed that each targeted base acquired the intended mutation. The cells edited with SEQ ID NO:3 and ABE8e showed only 4% wild type reads. The cells edited with SEQ ID NOs: 1 or 2 and BE4max showed about 9-12% wild type reads (FIG. 5B). Flow cytometry analysis of the edited cells with two antibodies that recognize an epitope located in exon 2 of CD33 confirmed the absence of CD33 exon 2 in the edited cells (FIG. 5C).

The edited cells were further analyzed for editing outcomes using cDNA and PCR with primers specific for CD33^{Δ2} (spanning exon junction 1-3), CD33FL (spanning exon junction 2-3 or exon 2), or common to all isoforms (in exon 3, spanning exon junction 3-5 or 4-5). The results showed that editing of ESE or SA induced exon 2 skipping without impacting the other exons (FIG. 5D).

### Example 2: CD33^{Δ2} Cells Display Normal Phagocytic Capacity and are Resistant to GO In Vitro

The CD34⁺CD33^{Δ2} cells as described in Example 1 were further analyzed.

### Phagocytosis

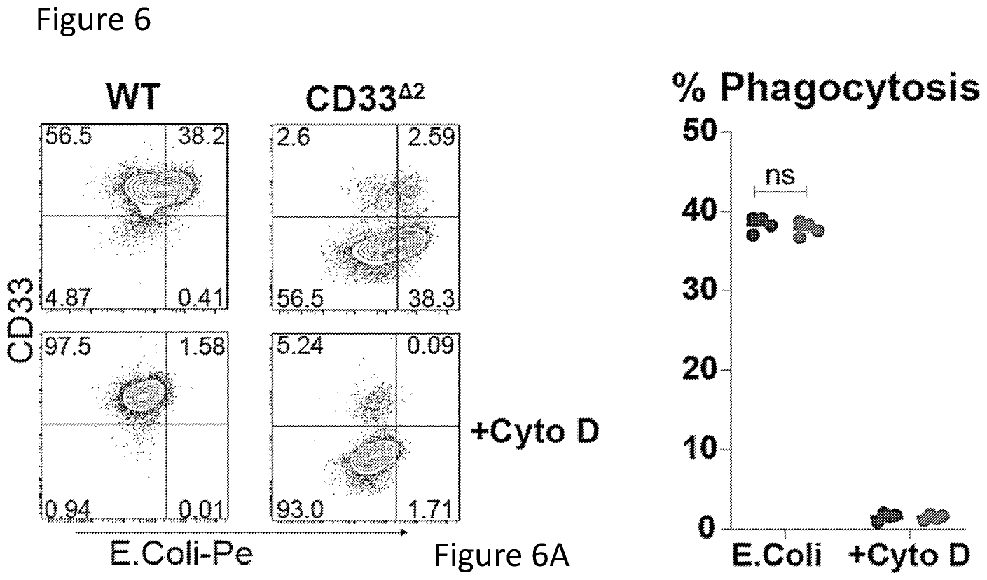

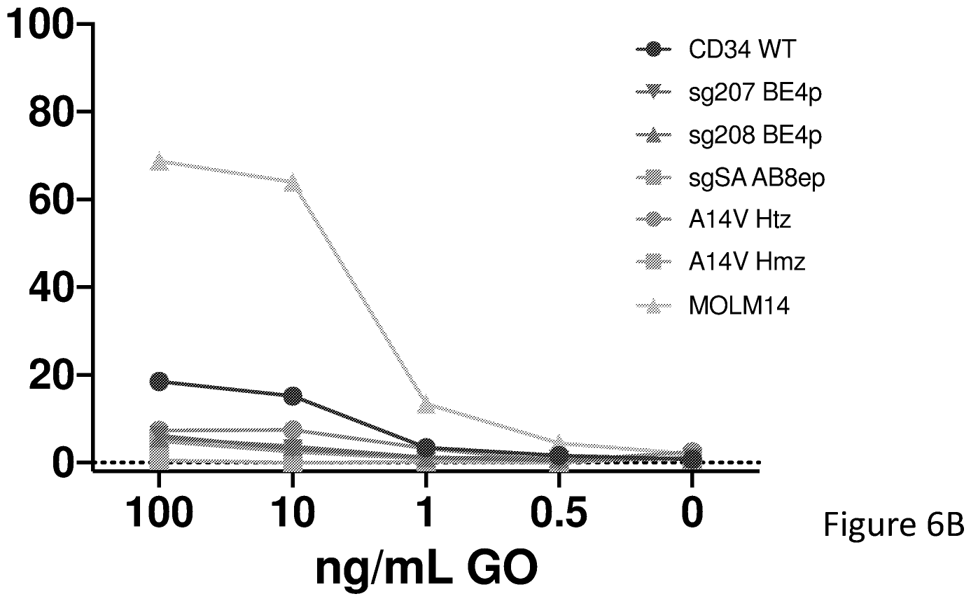

The ability of in vitro differentiated CD34⁺CD33^{WT} and CD33^{Δ2} monocytes to phagocytose E coli bioparticles in vitro was tested. In vitro differentiated WT or CD33^{Δ2} monocytes show comparable phagocytosis capacity, as measured by E. Coli bioparticles internalization. See FIG. 6A.

### GO Cytotoxicity

Additionally, CD34⁺CD33^{Δ2} resisted GO cytotoxicity in vitro. The cells were incubated with GO for 48 hours and analyzed by FACS using Sytox Blue or 7AAD as a viability dye. CD34⁺CD33^{Δ2} show same resistance to GO cytotoxicity than a donor with homozygous rs12459419 (TT) A14V SNP (FIG. 6B).

### Example 3: CD34⁺CD33^{Δ2} are Capable of Long Term Multilineage Engraftment and are Resistant to CD33-Targeted Immunotherapy (GO) In Vivo

### Materials and Methods

### In Vivo Experiments

Since present approach may involve transplanting CD33 gene-edited stem cells (CD34⁺CD33^{Δ2}) as a platform for CAR-T targeting CD33 antigen and ADC delivery (GO), it is important to test the ability of CD34⁺CD33^{Δ2} cells to engraft and contribute to myelopoiesis and lymphopoiesis.

NOD.Cg-Prkdc^{scid}Il2rg^{tm1Wjl}/SzJ (NSG) or NOD.Cg-Prkdc^{scid} Il2rg^{tm1Wjl} Tg(CMV-IL3, CSF2, KITLG)1Eav/MloySzJ (NSG-SGM3) mice (The Jackson Laboratory, Bar Harbor, Maine, USA) were conditioned with sublethal (100 cGy) total-body irradiation (TBI). CD34+WT or CD34⁺CD33^{Δ2} HSPCs (cells edited using the ABE8e and SEQ ID NO: 3 as described in Example 1) were injected into the sublethally irradiated mice via tail vein injection.

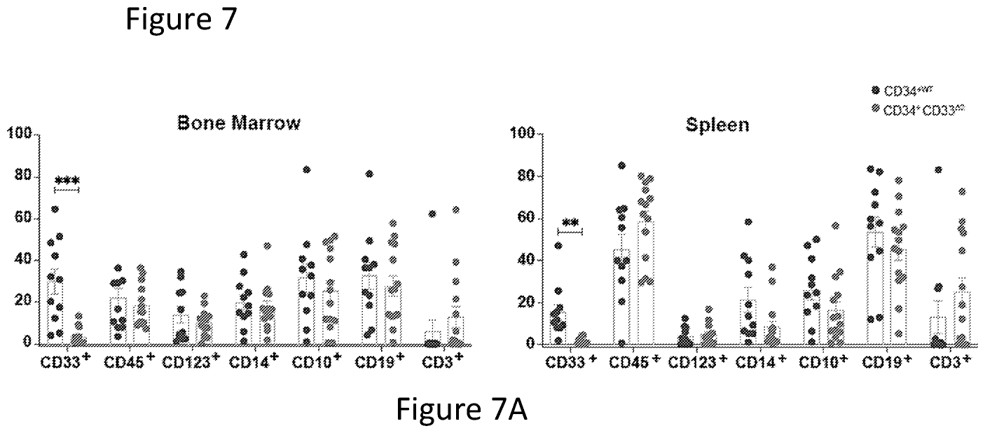

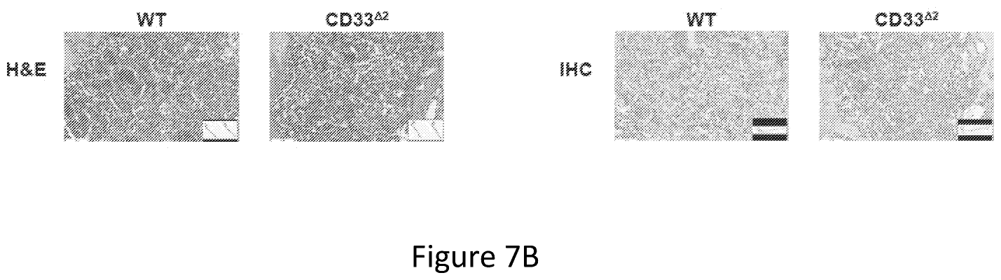

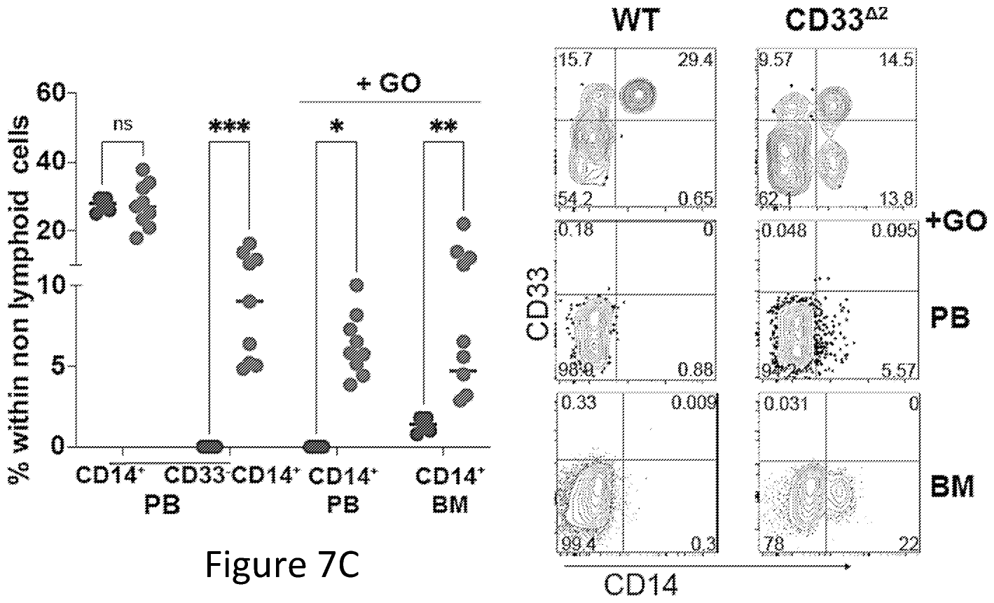

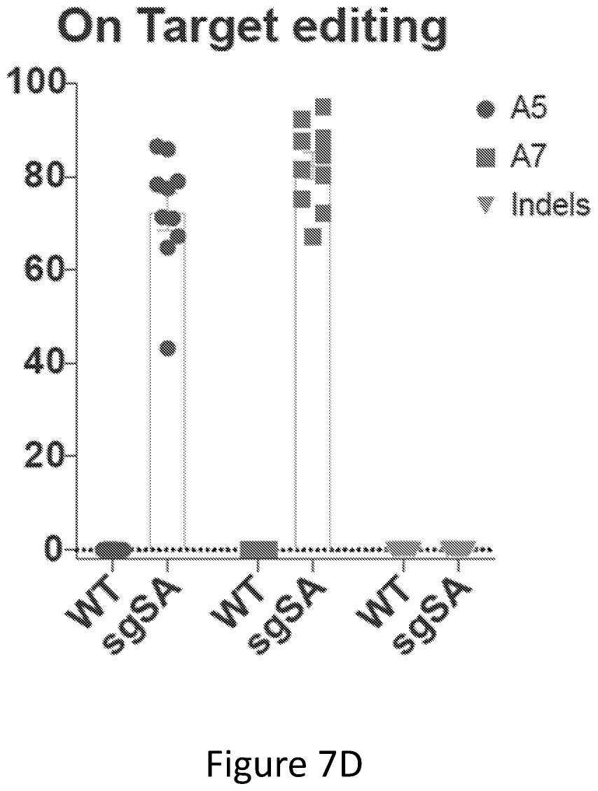

Engraftment and repopulation of the hematopoietic system over time (FIG. 7A)., was assessed by analysis of peripheral blood (PB), spleen, and whole bone marrow (BM) using the consequent antibodies from Biolegend (San Diego, CA, USA) or BD Biosciences (San Jose, CA, USA); Ter119-PeCy5, Ly5-BV711, H2kd-BV711, hCD45-BV510, hCD3-Pacific Blue, hCD123-BV605, hCD33-APC, hCD14-APC/Cy7, hCD10-BUV395, hCD19-BV650, CD34-BV421, CD90-PeCy7, hCD38-BUV661, and hCD45RA-BUV737. Dead cells were excluded using Propidium Iodide. CD34⁺ injected derived human cells were gated on Ter119-, Ly5-/H2kd-human CD45+.

To show the CD34⁺CD33^{Δ2} cells were resistant to GO in vivo, PB of 12 weeks post-transplanted mice was analyzed for the presence of CD33⁺ CD14⁺ cells or CD33^{Δ2}CD14⁺ cells. Mice were then injected with 2.5ugr GO then bled and sacked one week after treatment to assess the presence of myeloid cells in the PB and the bone marrow of the humanized mice. Before GO treatment, CD34⁺ WT or CD34⁺CD33^{Δ2} transplanted mice displayed same frequency of CD14⁺ cells in the PB (FIG. 7D, top FACS plots). One week, after GO injection while CD33⁻CD14⁺ cells were detected in the PB and BM of mice engrafted with CD34⁺CD33^{Δ2} cells, CD33⁺ and CD14⁺ cells had been eradicated in the PB and BM of CD34⁺WT engrafted mice.

Sixteen weeks after transplantation, the CD33 locus was amplified from genomic DNA from the bone marrow of the mice. Amplicons were sequenced by HTS and A-toG editing at position A7 was quantified.

All experiments were performed under protocols approved by the Institutional Animal Care and Use Committee of Columbia University.

### Results

Both bone marrow and spleen at 16 weeks post-transplant revealed the presence of human CD45⁺ cells and progenitors myeloid (CD123) and lymphoid (CD10), as well as mature myeloid (CD14) and lymphoid (CD19), and T cells (CD3) within the human CD45 (FIG. 7B). All cells remained CD33-negative (FIG. 7A).

CD33-CD14⁺ are detected in the peripheral blood and bone marrow of mice engrafted with CD34⁺CD33^{Δ2} cells after GO treatment, while CD33⁺ cells are undetectable, leading to eradication of the CD14⁺CD34^{+WT} engrafted mice. Therefore, CD33^{WT} cells remain sensitive to GO, while CD34⁺CD33^{Δ2} cells were insensitive. FIGS. 7B and 7C.

On-target editing at the target site (A7) in the engrafting WT (unedited) or edited cells was analyzed from bone marrow samples at 16 weeks post-transplantation. Sixteen weeks after transplantation, the CD33 locus was amplified from genomic DNA from the bone marrow of mice. FIG. 7D.

### Example 4: Off Target Assessments

### Materials and Methods

### CIRCLEseq

To identify base editing off target sites, genomic DNA was extracted from CD34+ enriched cell populations from de-identified human donors using the QIAgen Gentra PureGene kit (Cat. No. 158445). CIRCLE-seq was performed as previously described (Tsai et al, Nature Methods (2017) 14:607-14). Briefly, purified genomic DNA was sheared with a Covaris S2 instrument to an average length of 300 bp. The fragmented DNA was end repaired, A-tailed and ligated to a uracil-containing stem-loop adaptor, using KAPA HTP Library Preparation Kit, PCR Free (KAPA Biosystems). Adaptor ligated DNA was treated with Lambda Exonuclease (NEB) and E. coli Exonuclease I (NEB) and then with USER enzyme (NEB) and T4 polynucleotide kinase (NEB). Intramolecular circularization of the DNA was performed with T4 DNA ligase (NEB) and residual linear DNA was degraded by Plasmid-Safe ATP-dependent DNase (Lucigen). In vitro cleavage reactions were performed with 250 ng of Plasmid-Safe-treated circularized DNA, 90 nM of Cas9 nuclease (NEB), Cas9 nuclease buffer (NEB) and 90 nM of synthetic chemically modified sgRNA (Synthego), in a 100 μl volume. Cleaved products were A-tailed, ligated with a hairpin adaptor (NEB), treated with USER enzyme (NEB) and amplified by PCR with barcoded universal primers NEBNext Multiplex Oligos for Illumina (NEB), using Kapa HiFi Polymerase (KAPA Biosystems). Libraries were sequenced with 150 bp paired-end reads on an Illumina MiSeq instrument. CIRCLE-seq data analyses were performed using open-source CIRCLE-seq analysis software (https://github.com/tsailabSJ/circleseq) using default parameters. The human genome GRCh37 was used for alignment.

To assess off-target editing, the 19 top off-target sites nominated by CIRCLE-seq were sequenced in engrafted human WT (unedited cells) or edited cells from the bone marrow at 16 weeks post-transplantation.

For each off-target site, primers were designed to generate a 250-300 bp product including the aligned off-target binding site for the guide RNA, and appended adapters for Illumina sequencing. Following a secondary PCR to barcode each sample, products were pooled and sequenced using a 300 cycle v2 Illumina MiSeq kit. Amplicons were sequenced by HTS and A-to-G editing at position A7 was quantified.

To analyze editing outcomes in resulting fastq files, CRISPResso2 was used to align each read to the reference amplicon and quantify indels or base changes.

The CD34⁺CD33^{Δ2} cells edited using the ABE8e and SEQ ID NO: 3 as described in Example 1 were further analyzed.

### Results

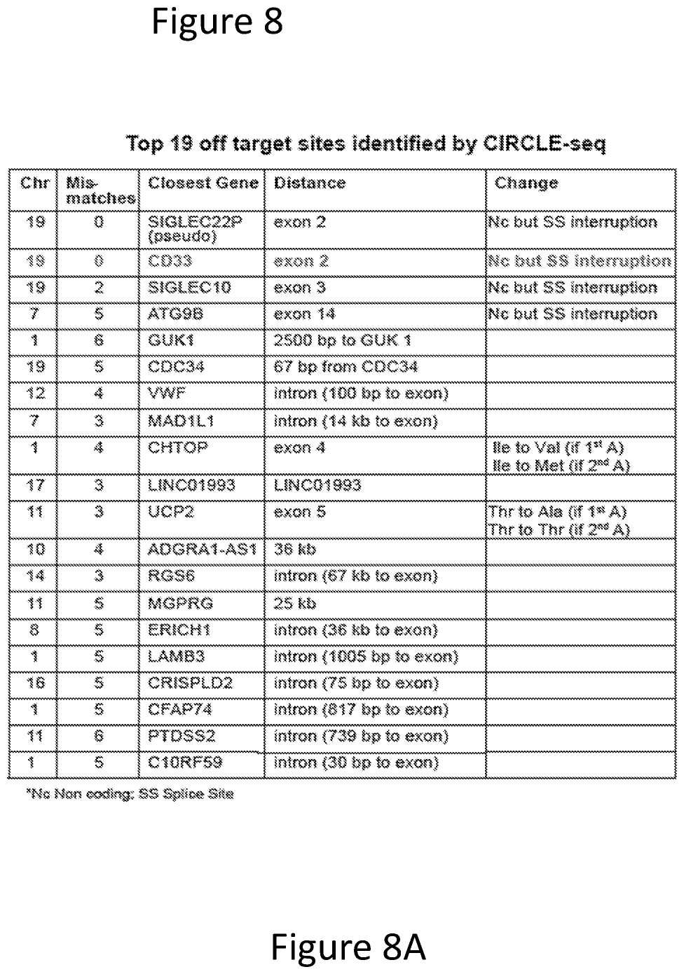

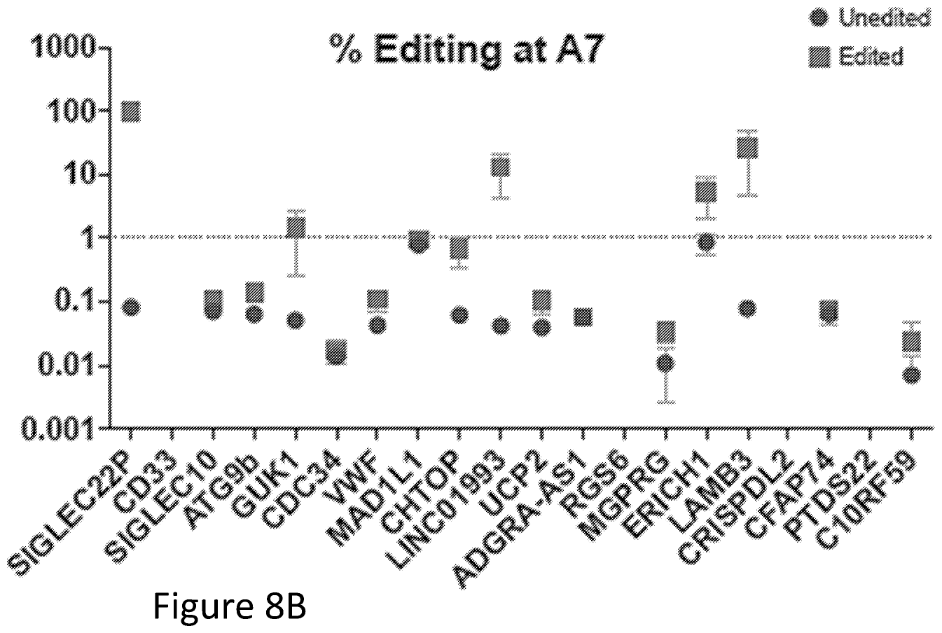

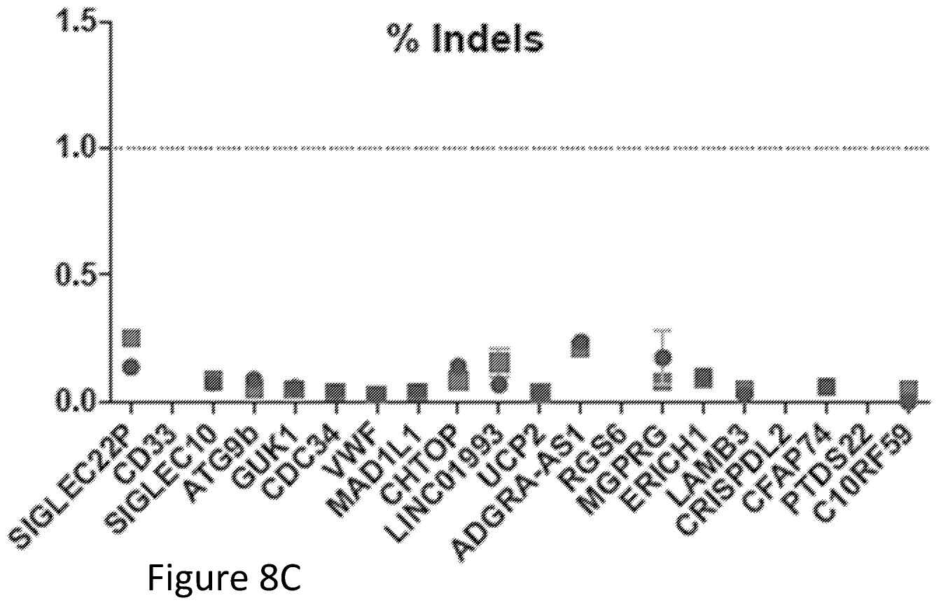

Using CIRCLE-seq, 19 top off target loci were identified. FIG. 8A.

As described above, sequencing was performed to assess A-to-G editing at position A7 editing at A7 targeted nucleotide at the 19 identified top off target loci in engrafting human WT (unedited) or edited cells. As shown in FIG. 8B, for most of the targets, the A7 editing was similar in WT and edited cells.

As shown in FIG. 8C, the percent of indels for the targets was also similar for both WT and edited cells.

### Example 5: Multiplexed Base Editing

Two sgRNAs were designed. One to induce exon skipping in CD33 using an ABE editor (SEQ ID NO: 3). The second was to induce skipping of exon 13 in EMR2 using an ABE editor (SEQ ID NO: 4). FIGS. 4A and 9A.

Two RNPs (one for each targeted gene) were separately prepared using 5ugr AB8e+1.5ugr sgRNA (chemically modified). After incubation, RNPs were comixed with 500,000 HSC/HSPCs and nucleofected using Lonza 4D nucleofector and program DZ100.

The edited cells were analyzed for the introduction of the nucleotide conversions. As shown by Sanger sequencing, the ABE base editor introduced the A>G conversions in both loci thereby editing the SA in the CD33 locus and the SD in EMR2 locus (FIG. 9B). Flow cytometry analysis of the edited cells confirmed that the editing abrogates CD33 and EMR2 antibodies binding in the edited cells (FIG. 9C).

### Example 6: Targeting Cell-Surface Lineage-Specific CD33 in Acute Myeloid Leukemia (AML)

The present example encompasses targeting of the CD33 antigen in AML. The specific steps of the example are outlined in Table 5.

TABLE 5
Outline of the Experimental Design

| | |
|---|---|
| I. Autologous CD33 targeted (CAR) T-cell | 1. Generation of anti-CD33 CAR constructs |
| therapy | 2. Isolation of CD8⁺T Cells from a Patient |
| | 3. Preparation of anti CD33 CAR T Cells |
| | 4. Reinfusion of CD33 CAR T cells into a Patient |
| II. Autologous Hematopoietic Stem Cell | 1. Isolation of Hematopoietic Stem Cells |
| Transplant Using CD34⁺CD33^{Δ2} Cells | 2. CRISPR base editing Plasmid Targeting CD33 |
| | 3. Generation of CD34⁺CD33^{Δ2} cells via |
| | CRISPR-based base editor system |
| | 4. Reinfusion of CD34⁺CD33^{Δ2} cells into a |
| | Patient |
| III. Continued treatment of a patient with a | |
| CD33 antibody attached to a toxin | |
| (immunotoxin) | |

I. CD33-Targeted Chimeric Antigen Receptor (CAR) T-Cell Therapy

A. Generation of anti-CD33 CAR constructs

The chimeric antigen receptors targeting CD33 described herein may consist of the following components in order from 5′ to 3′: pHIV-Zsgreen lentiviral backbone (www.addgcne.org/18121/), peptide signal, the CD33 scFv, the hinge, transmembrane regions of the CD28 molecule, the intracellular domain of CD28, and the signaling domain of TCR-ζ molecule.

Initially, peptide signal, anti-CD33 light chain (SEQ ID NO: 8), the flexible linker and the anti-CD33 heavy chain (SEQ ID. NO. 6) are cloned into the EcoRI site of pHIV-Zsgreen, with an optimal Kozak sequence.

The nucleic acid sequences of an exemplary chimeric receptors that binds CD33 with the basic structure of Light chain—linker—Heavy chain—Hinge-CD28/ICOS-CD3ζ is provided below.

Part 1: Light chain—linker-Heavy chain (SEQ ID NO: 33): The Kozak start site is shown in boldface. The peptide signal L1 is shown in italic. The anti-CD33 light chain and heavy chain are shown in bold and italics, separated by a linker.

ggtgtcgtgagcggccgctgaactgGCCACCATGGACATG
AGGGTCCCTGCTCAGCTCCTGGGGCTCCTGCTGCTCTGGC
TCTCAGGTGCCAGATGTGAGATCGTGCTGACCCAGAGCCC
CGGCAGCCTGGCCGTGAGCCCCGGCAAGAGGGTGACCATG
AGCTGCAAGAGCAGCCAGAGCGTGTTCTTCAGCAGCAGCC
AGAAGAACTACCTGGCCTGGTACCAGCAGATCCCCGGCCA
GAGCCCCAGGCTGCTGATCTACTGGGCCAGCACCAGGGAG
AGCGGCGTGCCCGACAGGTTCACCGGCAGCGGCAGCGGCA
GCGGCACCGACTTCACCCTGACCATCAGCAGCGTGCAGCC
CGAGGACCTGGCCATCTACTACTGCCACCAGTACCTGAGC
AGCAGGACCTTCGGCCAGGGCACCAAGCTGGAGATCAAGA
GGGGCAGCACCAGCGGCAGCGGCAAGCCCGGCAGCGGCGA
GGGCAGCACCAAGGGCCAGGTGCAGCTGCAGCAGCCCGGC
GCCGAGGTGGTGAAGCCCGGCGCCAGCGTGAAGATGAGCT
GCAAGGCCAGCGGCTACACCTTCACCAGCTACTACATCCA
CTGGATCAAGCAGACCCCCGGCCAGGGCCTGGAGTGGGTG
GGCGTGATCTACCCCGGCAACGACGACATCAGCTACAACC
AGAAGTTCCAGGGCAAGGCCACCCTGACCGCCGACAAGAG
CAGCACCACCGCCTACATGCAGCTGAGCAGCCTGACCAGC
GAGGACAGCGCCGTGTACTACTGCGCCAGGGAGGTGAGGC
TGAGGTACTTCGACGTGTGGGGCCAGGGCACCACCGTGAC
CGTGAGCAGC

Part 2: Hinge-CD28/ICOS-CD3 (NotI restriction enzyme recognition sites are shown in capitalization. The translational stop site is in boldface. The BamHI restriction cleavage site is shown in underline.

CD28 costimulatory domain
(SEQ ID NO: 34)
GCGGCCGCAattgaagttatgtatcctcctccttacctag
acaatgagaagagcaatggaaccattatccatgtgaaagg
gaaacacctttgtccaagtcccctatttcccggaccttct
aagcccttttgggtgctggtggtggttggtggagtcctgg
cttgctatagcttgctagtaacagtggcctttattatttt
ctgggtgaggagtaagaggagcaggctcctgcacagtgac
tacatgaacatgactccccgccgccccgggcccAcccgca
agcattaccagccctatgccccaccacgcgacttcgcagc
ctatcgctccagagtgaagttcagcaggagcgcagacgcc
cccgcgtaccagcagggccagaaccagctctataacgagc
tcaatctaggacgaagagaggagtacgatgttttggacaa
gagacgtggccgggaccctgagatggggggaaagccgaga
aggaagaaccctcaggaaggcctgtacaatgaactgcaga
aagataagatggcggaggcctacagtgagattgggatgaa
aggcgagcgccggaggggcaaggggcacgatggcctttac
cagggtctcagtacagccaccaaggacacctacgacgccc
ttcacatgcaggccctgccccctcgcTAAcgcccctctcc
ctcccccccccctaa
ICOS costimulatory domain
(SEQ ID NO: 35)
GCGGCCGCActatcaatttttgatcctcctccttttaaag
taactcttacaggaggatatttgcatatttatgaatcaca
actttgttgccagctgaagttctggttacccataggatgt
gcagcctttgttgtagtctgcattttgggatgcatactta
tttgttggcttacaaaaaagaagtattcatccagtgtgca
cgaccctaacggtgaatacatgttcatgagagcagtgaac
acagccaaaaaatctagactcacagatgtgaccctaagag
tgaagttcagcaggagcgcagacgcccccgcgtaccagca
gggccagaaccagctctataacgagctcaatctaggacga
agagaggagtacgatgttttggacaagagacgtggccggg
accctgagatggggggaaagccgagaaggaagaaccctca
ggaaggcctgtacaatgaactgcagaaagataagatggcg
gaggcctacagtgagattgggatgaaaggcgagcgccgga
ggggcaaggggcacgatggcctttaccagggtctcagtac
agccaccaaggacacctacgacgcccttcacatgcaggcc
ctgccccctcgcTAAcgcccctctccctccccccccccta
a
Fusion (hybrid) CD28 and ICOS
costimulatory domain
(SEQ ID NO: 36)
GCGGCCGCAattgaagttatgtatcctcctccttacctag
acaatgagaagagcaatggaaccattatccatgtgaaagg
gaaacacctttgtccaagtcccctatttcccggaccttct
aagcccttttgggtgctggtggtggttggtggagtcctgg
cttgctatagcttgctagtaacagtggcctttattatttt
ctgggtgaggagtaagaggagcaggctcctgcacagtgac
tacatgttcatgagagcagtgaacacagccaaaaaatcta
gactcacagatgtgaccctaagagtgaagttcagcaggag
cgcagacgcccccgcgtaccagcagggccagaaccagctc
tataacgagctcaatctaggacgaagagaggagtacgatg
ttttggacaagagacgtggccgggaccctgagatgggggg
aaagccgagaaggaagaaccctcaggaaggcctgtacaat
gaactgcagaaagataagatggcggaggcctacagtgaga
ttgggatgaaaggcgagcgccggaggggcaaggggcacga
tggcctttaccagggtctcagtacagccaccaaggacacc
tacgacgcccttcacatgcaggccctgccccctcgcTAAc
gcccctctccctcccccccccctaa

In the next step, the hinge region, CD28 domain (SEQ ID NO: 15) and a cytoplasmic component of TCR-ζ are cloned into the NotI and BamHI sites of pHIV-Zsgreen (already containing the peptide signal and the CD33 scFv. Alternatively, CD28 domain can be substituted by ICOS domain (SEQ ID NO: 16).

In addition to CD28 and ICOS domains, a fusion domain comprising fragments of CD28 and ICOS intracellular signaling domains will be engineered (SEQ ID NO: 17) and used to generate additional chimeric receptors. Such configuration, where the chimeric receptor comprises an antigen-binding fragment, an anti-CD33 light chain variable region, a linker, an anti-CD33 heavy chain variable region, CD28/ICOS hybrid region (including a TM region of CD28) and signaling domain of TCR-(molecule.

Example amino acid sequences of components that may be used to generate the chimeric receptors are provided herein, such as CD28 domain (SEQ ID NO: 10), ICOS domain (SEQ ID NO: 11), CD28/ICOS hybrid domain (SEQ ID NO: 13), and TCR-ζ are provided herein. Alternatively, the chimeric receptor may be generated as well (Section B.)

B. Alternative Method for Generation of Anti-CD33 CAR Constructs

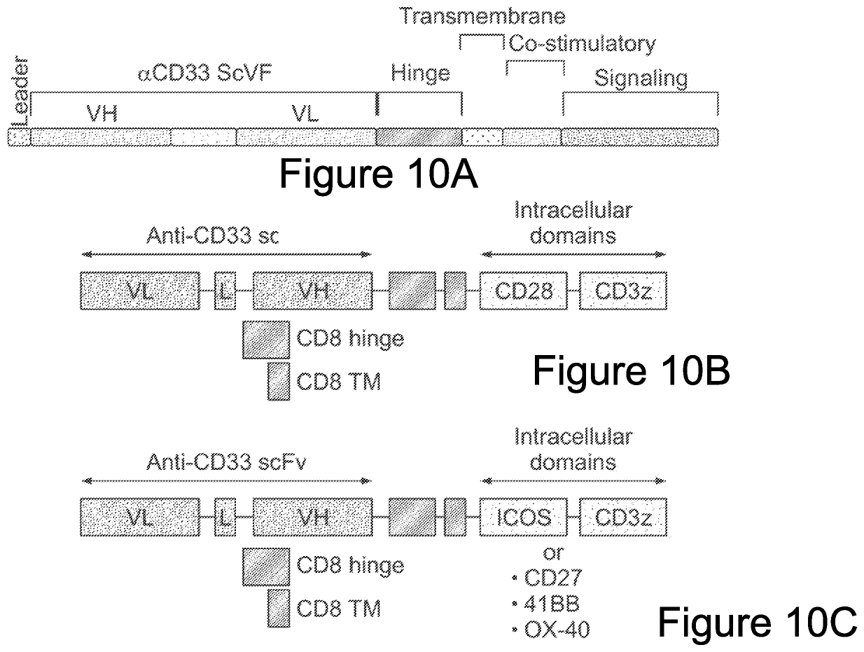

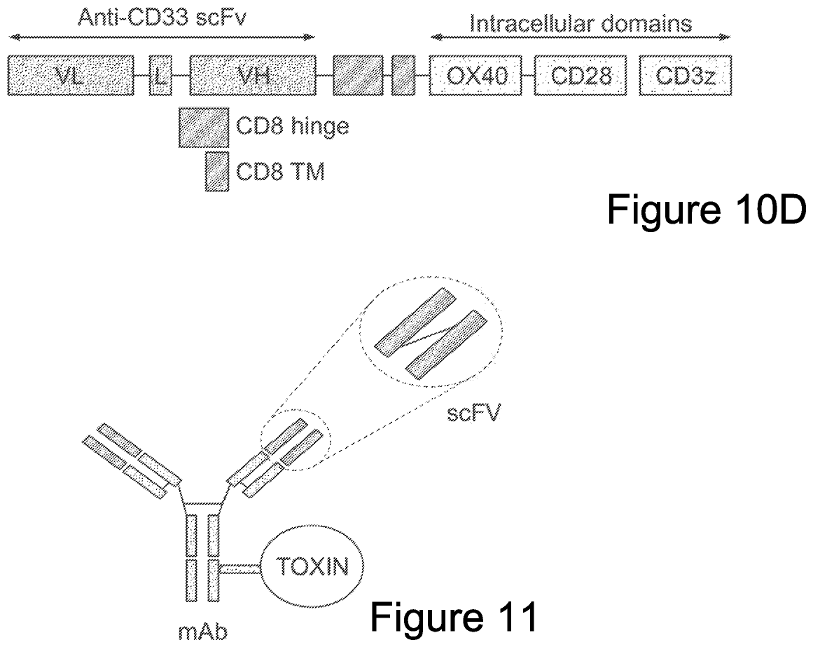

Schematics of example chimeric receptors are presented in FIG. 10, panels A-D. The chimeric receptor will be generated using an extracellular humanized scFv recognizing the CD33 antigen, linked to an extracellular CD8 hinge region, a transmembrane and cytoplasmic signaling domain, and a CD3 ζ-signaling chain (FIG. 10, panel B). DNA encoding the anti-CD33 chimeric receptor will be generated by using a humanized scFv (Essand et al., J Intern Med. (2013) 273(2):166). Alternatives include a CAR T cell that contains OX-1 or 41-BB in place of CD28 or CD28/OX1 or CD28/4-1-B-B hybrids (FIG. 10, panels C and D).

In order to generate the anti-CD33 scFV sequence, the coding regions of the heavy and light chains of the variable regions of the anti-CD33 antibody described above (SEQ ID NOs: 6 and 8) are amplified with specific primers and cloned into a pHIV-Zsgreen vector for expression in cells. To evaluate the binding strength of the scFv (single chain variable fragments) to the target antigen, the scFv is expressed in Hek293T cells. For this purpose, the vector (pHIV-Zsgreen containing the coding areas) are transformed into E. coli Top10F bacteria and the plasmids prepared. The obtained expression vectors that code for the scFv antibodies are introduced by transfection into Hek293T cells. After culturing the transfected cells for five days, the supernatant is removed and the antibodies purified.

The resulting antibodies can be humanized using framework substitutions by protocols known in the art. See, for example, one such protocol is provided by BioAtla (San Diego), where synthetic CDR encoding fragment libraries derived from a template antibody are ligated to human framework region encoding fragments from a human framework pool limited to germline sequences from a functionally expressed antibodies (bioatla.com/applications/express-humanization/).

Affinity maturation may be performed in order to improve antigen binding affinity. This can be accomplished using general techniques known in the art, such as phage display (Schier R., J. Mol. Biol (1996), 263:551). The variants can be screened—for their biological activity (e.g., binding affinity) using for example Biacore analysis. In order to identify hypervariable region residues which would be good candidates for modification, alanine scanning mutagenesis can be performed to identify hypervariable region residues contributing significantly to antigen binding. Additionally, combinatorial libraries described by can also be used for improving the affinity of the antibodies (Rajpal et al., PNAS (2005) 102(24): 8466). Alternatively, BioAtla has developed a platform for the rapid and efficient affinity maturation of antibodies, which can also be utilized for the purposes of antibody optimization (bioatla.com/applications/functional-maturation/).

C. Assembly of CAR Construct

Next, the anti-CD33 scFv is linked to an extracellular CD8 hinge region, a transmembrane and cytoplasmic CD28 signaling domain, and a CD3 ζ-signaling chain. Briefly, primers specific for anti-CD33 scFv sequence are1 be used to amplify the scFv as described above. Plasmid (pUN1-CD8). (www.invivogen.com/puno-cd8a) carrying the complete human CD8 coding sequence will be used to amplify CD8 hinge and transmembrane domains (amino acids 135-205). CD3 ζ fragment is amplified from the Invivogen plasmid pORF9-hCD247a (http://www.invivogen.com/PDF/pORF9-hCD247a_10E26v06.pdf) carrying the complete human CD3ζ coding sequence. Finally, the CD28 (amino acids 153-220, corresponding to TM and signaling domains of CD28) is amplified from cDNA generated using RNA collected from activated T cells by Trizol method. Fragments containing anti-CD33-scFv-CD8-hinge+TM-CD28-CD3ζ are assembled using splice overlap extension (SOE) PCR. The resulting PCR fragment is cloned into pELPS lentiviral vector. pELPS is a derivative of the third-generation lentiviral vector pRRL-SIN-CMV-eGFP-WPRE in which the CMV promoter was replaced with the EF-1α promoter and the central polypurine tract of HIV was inserted 5′ of the promoter (Milone et al., Mol Ther. (2009) (8): 1453, Porter et al., NEJM (2011) (8):725). All constructs are verified by sequencing.

Alternatively, CARs containing ICOS, CD27, 41BB, or OX-40 signaling domain instead of CD28 domain will be generated, introduced into T-cells and tested for the ability to eradicate CD33 positive cells (FIG. 10, panel C). The generation of “third-generation” chimeric receptors are also contemplated (FIG. 10, panel D), which combine multiple signaling domains, such as CD3z-CD28-41BB or CD3z-CD28-OX40, to further augment potency (Sadelain et al., Cancer Discov. (2013) 4:388).

D. Anti-CD33 CAR T Cell Preparation

Primary human CD8⁺ T cells are isolated from patients' peripheral blood by immunomagnetic separation (Miltenyi Biotec). T cells are cultured in complete media (RPMI 1640 supplemented with 10% heat-inactivated FBS, 100 U/mL penicillin, 100 μg/mL streptomycin sulfate, 10 mM HEPES) and stimulated with anti-CD3 and anti-CD28 mAbs-coated beads (Invitrogen) as previously described (Levine et al., J. Immunol. (1997) 159(12):5921).

A packaging cell line is used to generate the viral vector, that is able to transduce target cells and contains the anti-CD33 chimeric receptors. To generate lentiviral particles, CARs generated in section (1) of this Example are transfected into immortalized normal fetal renal 293T packaging cells. Cells are cultured with high glucose DMEM, including 10% FBS, 100 U/ml penicillin and 100 μg/ml streptomycin. 48-72 hours post-transfection the supernatant are collected, and the recombinant lentivirus concentrated in DMEM without FBS. Primary CD8⁺ T cells are next transduced at multiplicity of infection (MOI) of ˜5-10 in the presence of polybrene. Human recombinant IL-2 (R&D Systems) is added every other day (50 IU/mL). T cells are cultured for ˜14 days after stimulation. Transduction efficiency of human primary T cells is assessed by expression of a ZsGreen reporter gene (Clontech, Mountain View, CA).

E. Infusion of CAR T Cells into a Patient

Prior to the i.v. infusion of anti-CD33 CAR T cells into the patient, cells are washed with phosphate buffered saline and concentrated. A cell processor such as a Haemonetics CellSaver (Haemonetics Corporation, Braintree, MA), which provides a closed and sterile system, is used for the washing and concentration steps before formulation. The final T cells expressing the anti-CD33 chimeric receptors will be formulated into 100 ml of sterile normal saline supplemented with human serum albumin. Finally, patients are infused with 1-10×10⁷ T cells/kg over a period of 1-3 days (Maude et al., NEJM (2014) 371(16):1507). The number of T cells expressing anti-CD33 chimeric receptors infused will depend on numerous factors such as the state of the cancer patient, patient's age, prior treatment, etc.

Furthermore, also contemplated herein are immune cells expressing chimeric receptors that target EMR2 in addition to chimeric receptors that target CD33 in AML patients. This can be accomplished by two different approaches: 1) generating immune cells expressing anti-CD33 chimeric receptors and immune cells expressing anti-EMR2 chimeric receptors separately and infusing the patient with both types of immune cells separately; or 2) generating immune cells that target both CD33 and EMR2 simultaneously (Kakarla et al., Cancer (2014) 2:151).

II. Autologous Hematopoietic Stem Cell Transplant (HSCT) Using CD34⁺ CD33″ Cells

It is understood that the protocols regarding stem cell isolation from patients, conditioning regimens, as well as infusion of patients with stem cells vary greatly depending on the patient's age, condition, treatment history, and institution where the treatment is conducted. Thus, the protocol described below is merely an example and is subject to routine optimization by a person having ordinary skill in the art.

A. Isolation of Hematopoietic Stem Cells Using Peripheral Blood Stem Cell (PBSC) Mobilization Following Adoptive Transfer of anti-CD33 CAR T Cells

AML patient will be stimulated by i.v. administration of granulocyte colony-stimulating factor (G-CSF) 10 mg/kg per day. CD34⁺ cell positive selection will be performed using immunomagnetic beads and an immunomagnetic enrichment device. A minimum of 2×10⁶ CD34⁺ cells/kg body weight are expected to be collected using a Fenwall CS 3000+ cell separator (Park et al., Bone Marrow Transplantation (2003) 32:889).

B. Conditioning Regimen of a Patient

The conditioning regimen for autologous peripheral blood stem cell transplant (PBSCT) will be carried out using etoposide (VP-16)+cyclophosphamide (CY)+total body irradiation (TBI). Briefly, the regimen will consist of etoposide (VP-16) at 1.8 g/m² i.v. constant infusion (c.i.v.) over 26 h as a single dose followed by cyclophosphamide (CY) at 60 mg/kg per day i.v. over 2 h for 3 days, followed by total body irradiation (TBI) at 300 cGy per day for the next 3 days.

To calculate the dose, ideal body weight or actual body weight, whichever is less, will be used. As previously mentioned, factors such as the state of the cancer patient, patient's age, prior treatment, as well as the type of institution where the procedure is conducted will all be taken into consideration when determining the precise conditioning regimen.

C. Plasmid Construction of CRISPR-based base editor system targeting CD33

A lentiviral vector containing a Cas nickase fused to an adenosine base editor (e.g., ABE8e) is obtained and a gRNA targeting CD33 (e.g., SEQ ID NO: 3) is cloned into the vector. CD33 gRNA oligonucleotides will be obtained from Integrated DNA Technologies (IDT), phosphorylated using polynucleotide kinase (Fermentas) at 37° C. for 30 minutes and annealed by heating to 95° C. for 5 minutes and cooling to 25° C. at 1.5° C./minute. T7 ligase will be used to anneal the oligos, after which the annealed oligos will be ligated into gel purified vector (Qiagen) at 25° C. for 5 minutes. Resulting plasmid can then be amplified using an endotoxin-free midi-prep kit (Qiagen) (Sanjana et al., Nat Methods (2014)(8):783).

Alternatively, a two vector system may be used (where gRNA and Cas are expressed from separate vectors) protocol described previously (Mandal et al., Cell Stem Cell (2014) 15(5):643). Here, Mandal et al. achieved efficient ablation of genes in human hematopoietic stem cells using CRISPR-Cas system expressed from non-viral vectors.

Also alternatively, a Cas nickase fused to an adenosine base editor (e.g., ABE8e) in protein form and the gRNA are delivered to the cell using a ribonucleoprotein (RNP) based delivery system.

In addition to infusing patients with CD33 depleted hematopoietic stem cells HSCs, a protocol infused with CD34⁺CD33^{Δ2} EMR^{Δ13} depleted HSCs using the methods of Example 5.

D. Transfection of CD34⁺ cells HSCs to generate CD34⁺CD33^{Δ2} or CD34⁺CD33^{Δ2} EMR^{Δ13-} Cells

Freshly isolated peripheral blood-derived CD34⁺ cells (from step 4) will be seeded at 1×10⁶ cells/ml in serum-free CellGro SCGM Medium in the presence of cells culture grade Stem Cell Factor (SCF) 300 ng/ml, FLT3-L 300 ng/ml, Thrombopoietin (TPO) 100 ng/ml and IL-3 60 ng/ml. Following 24 hour of pre-stimulation, CD34⁺ HSCs will be transfected with LentiCRISPR v2 containing Cas9 and CD33 gRNA using Amaxa Human CD34 cell Nucleofector kit (U-008) (#VPA-1003) (Mandal et al., Cell Stem Cell (2014) 15(5):643) or using a ribonucleoprotein (RNP) based delivery system. 24-48 hours post-transfection, CD34⁺CD33^{Δ2} or CD34⁺CD33^{Δ2} EMR A¹³ cells are selected with 1.2 μg/ml puromycin. Following the puromycin selection, cells will be maintained in puromycin-free media for couple of days.

E. Reinfusion of CD34⁺ CD33^{Δ2} Cells into the Patient

CD34⁺ cells transfected ex vivo with CRISPR-based base editor system (CD34⁺CD33^{Δ2} or CD34⁺CD33^{Δ2} EMR^{Δ13} cells) are immediately reinfused through a Hickman catheter using a standard blood administration set without a filter (Hacein-Bey Abina et al. JAMA (2015) 313(15):1550).

Generally, patients who have undergone the above outlined treatment protocol will be monitored for the reappearance of circulating blasts and cytopenias. Additionally, depending on the underlying mechanism of AML in a specific patient, the success of the treatment will be monitored by testing for reappearance of an informative molecular or cytogenetic marker, or an informative flow cytometry pattern. For example, reemergence of a BCR-ABL signal in Philadelphia chromosome-positive AML will be detected using fluorescent in situ hybridization (FISH) with probes for BCR (on chromosome 22) and ABL (on chromosome 9).

To evaluate the success of CD33 deletion via CRISPR-based base editor system, peripheral blood CD34⁺ cells will be isolated from patients (post-transplant) and assessed for the CD33 expression, for example using flow cytometry, Western blotting, or immunohistochemistry.

As described herein, the HSCT described in this Example can be either autologous or allogeneic, and both approaches are suitable and can be incorporated in the methods described in the present disclosure.

III. Optional Step: Continued Treatment of a Patient with a CD33 Antibody Attached to a Toxin

A. Treatment of Patients with CD33 Immunotoxin Gemtuzumab Ozogamicin (GO)

Patients will be treated with 9 mg/m² of anti-CD33 antibody gemtuzumab ozogamicin (GO) as a 2-hour intravenous infusion in 2 doses separated by 2 weeks (Larson et al., Cancer (2005) 104(7):1442-52). GO is comprised of a humanized monoclonal antibody against CD33 which is conjugated with a cytostatic agent, calicheamicin (FIG. 11).

Alternatively, the anti-CD33 antibodies may be conjugated to different toxins, such as diphtheria toxin, Pseudomonas exotoxin A (PE), or ricin toxin A chain (RTA) can be generated (Wayne et al., Blood (2014) 123(16): 2470). Similarly, anti-EMR2 antibodies may be attached to a toxin and included in the treatment regimen.

### Example 7: T Cells and NK Cell Lines Expressing an Anti-CD33 Chimeric Receptor Induce Cell Death of Target Cells Expressing CD33

### Binding of Chimeric Receptors to CD33

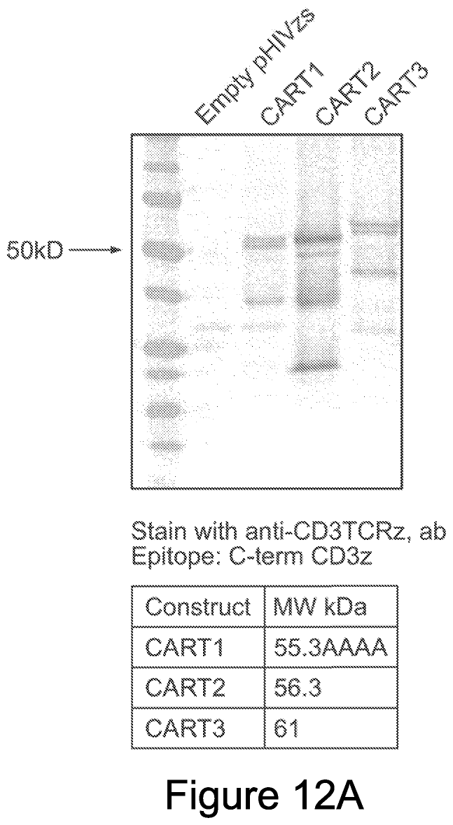

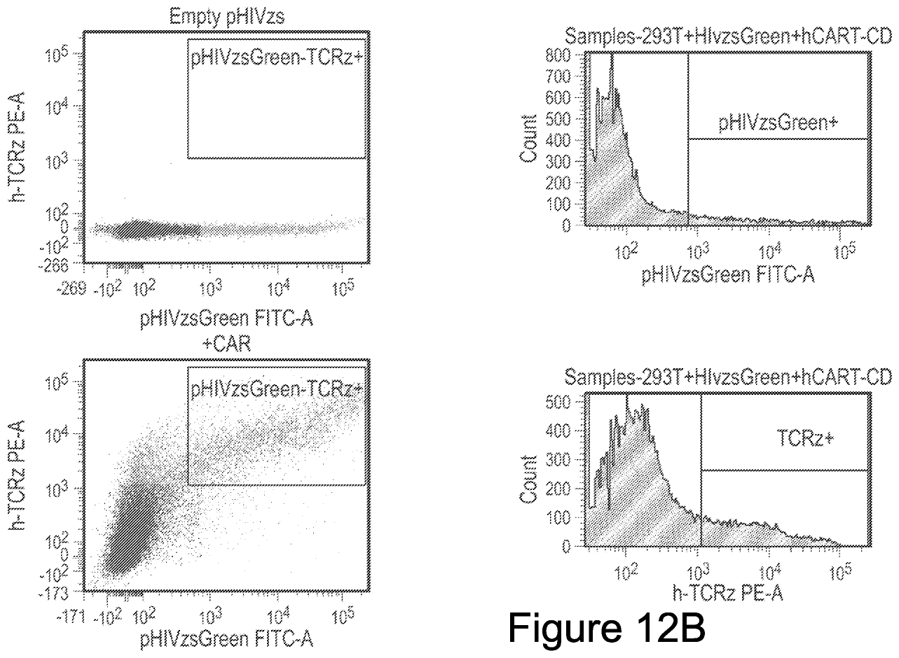

Chimeric receptors that bind CD33 (e.g., CART1, CART2, CART3) were generated using convention recombinant DNA technologies and inserted into a pHIV-Zsgreen vector (Addgene; Cambridge, MA). The vectors containing the chimeric receptors were used to generate lentiviral particles, which were used to transduce different cell types, for example T cell lines (e.g., 293 T cells) and NK cell lines (e.g., NK92 cells). Expression of the chimeric receptors was detected by Western blotting (FIG. 12, panel A) and flow cytometry (FIG. 12, panel B).

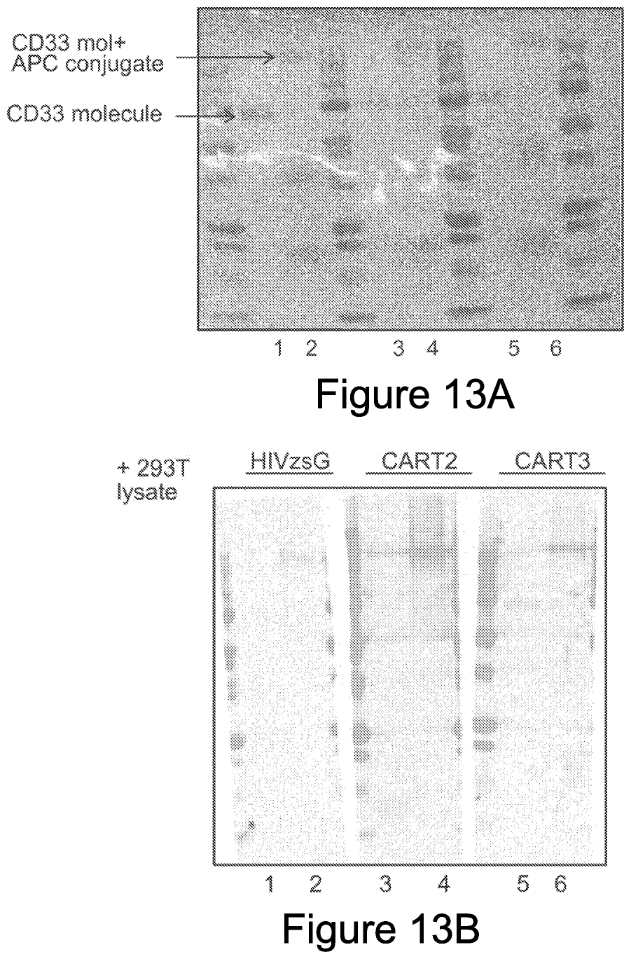

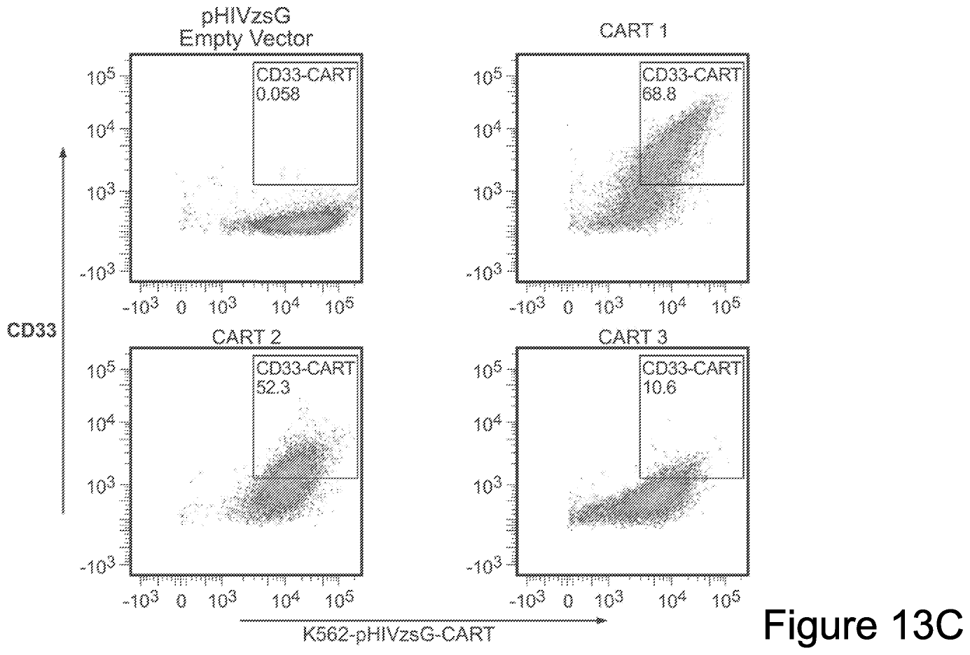

Cells expressing the chimeric receptors were selected by fluorescence-activated cell sorting (FACS) and assessed for their ability to bind CD33. Briefly, lysates of 293T cells expressing the chimeric receptors were coincubated with CD33 or CD33-allophycocyanin (APC) conjugate. The samples were subjected to protein electrophoresis and either stained with Ponceau protein stain (FIG. 13, panel A) or transferred to a membrane and probed with an anti-CD3ζ primary antibody (FIG. 13, panel B). In both cases, binding between the chimeric receptors and their target, CD33.

K562 cells expressing the chimeric receptors were also assessed for binding to CD33 by flow cytometry using CD33 as a probe (FIG. 13, panel C). There was an increase in the number of cells positive for expression of the chimeric receptor (CART1, CART2, or CART3) and CD33 binding as compared to cells containing an empty vector control, indicating the chimeric receptors bind to CD33.

### Cytotoxicity Induced by Cells Expressing the Chimeric Receptors

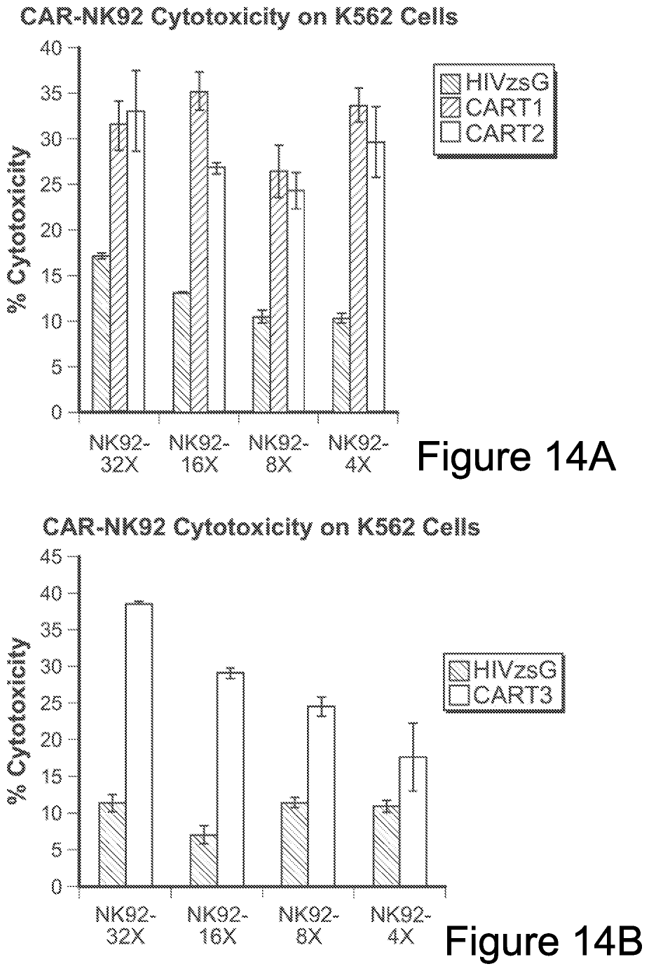

NK-92 cells expressing the chimeric receptors were functionally characterized for the ability to induce cytotoxicity of target cells presenting CD33 on the cell surface (e.g., K562 are a human chronic myelogenous leukemia cell line that are CD33⁺). To perform the cytotoxicity assays, effector cells (immune cells, such as NK-92 cells) were infected with lentivirus particles encoding the chimeric receptors and expanded. Seven days post infection, cells expressing the chimeric receptors were selected by FACS analysis by selecting for fluorescent markers also encoded by the chimeric receptor encoding vector (e.g., GFP+ or Red+). The selected cells that express the chimeric receptors were expanded for one week. Fourteen days post infection, the cytotoxicity assay was performing involving staining the target cells (cells expressing the target cell-surface lineage-specific antigen, CD33) with carboyxfluorescein succinimidyl ester (CFSE) and counting both the target cells and cells expressing the chimeric receptors. Different ratios of target cells and cells expressing the chimeric receptors were coincubated in round bottom 96-well plates for 4.5 hrs, after which 7-aminoactinomycin D (7-AAD) was added to stain non-viable cells. Flow cytometry was performed to enumerate the population of viable and non-viable target cells. As shown in FIG. 14, panels A and B, NK92 cells expressing chimeric receptors CART1, CART2, or CART3 induced a substantial amount of cell death of target K562 cells at each of the cell ratios assessed.

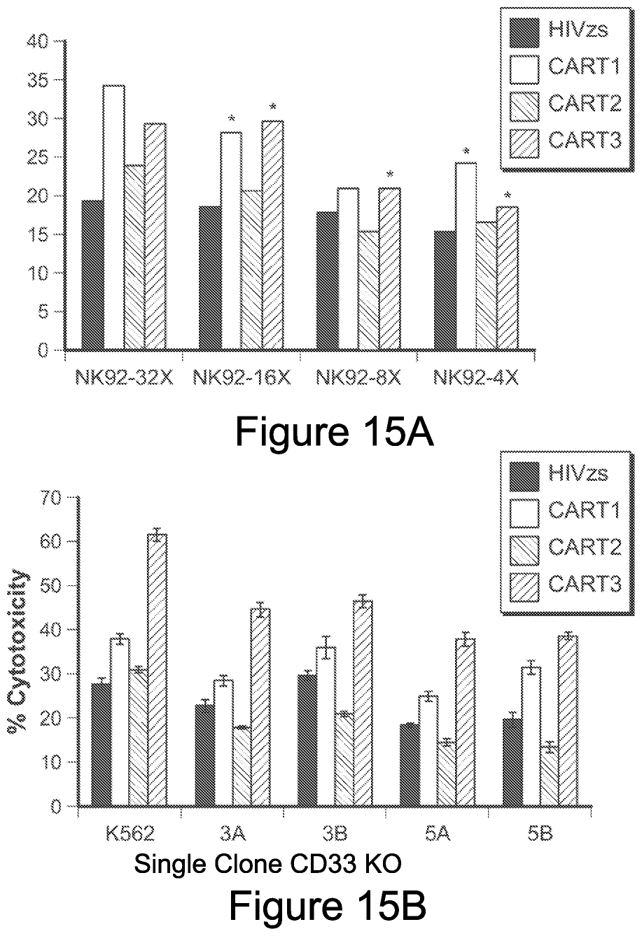

To determine that the cell death of K562 cells was dependent on specific targeting of the chimeric receptor to CD33, K562 were genetically engineered to be deficient in CD33 using a CRISPR/Cas system. Briefly, a human codon-optimized Cas9 endonuclease and a gRNA targeting a portion of the IgC domain of CD33 were expressed in the K562 cells, resulting in populations of CD33-deficient K562 cells. The cells were expanded and co-incubated with NK92 cells expressing the chimeric receptors, and the cytotoxicity assay was performed as described above. As shown in FIG. 15 panel A, the pooled CD33-deficient K562 cells showed a modest reduction in cell death with co-incubated with the NK92 cells expressing the chimeric receptors. However, when single clones of CD33-deficient K562 cells were isolated, expanded, and used to perform the cytotoxicity assays, a more significant reduction in cytotoxicity was observed (FIG. 15, panel B).

### Expression of Chimeric Receptors in Primary T Cells

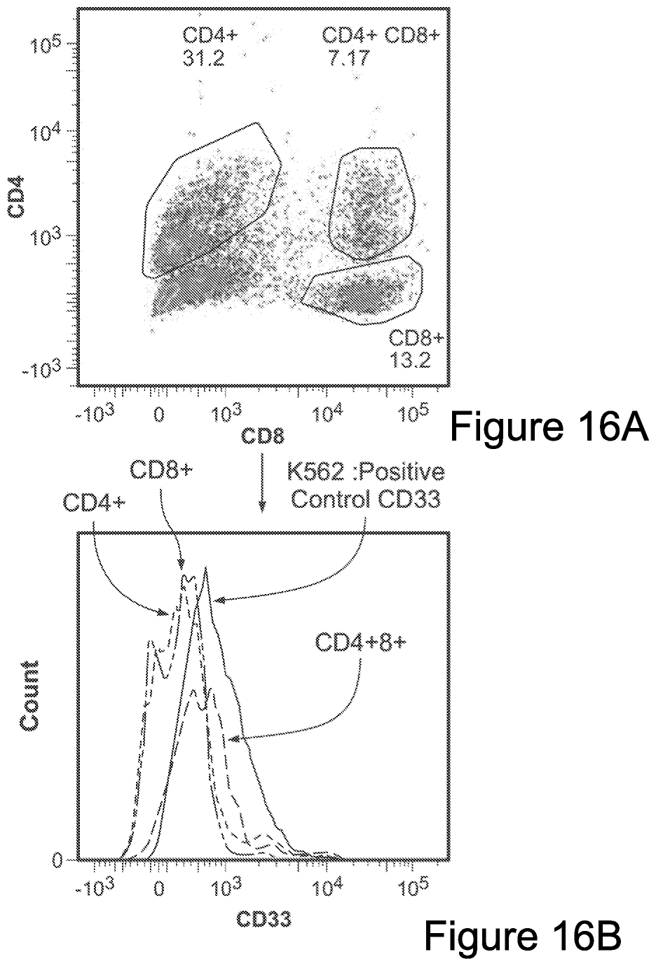

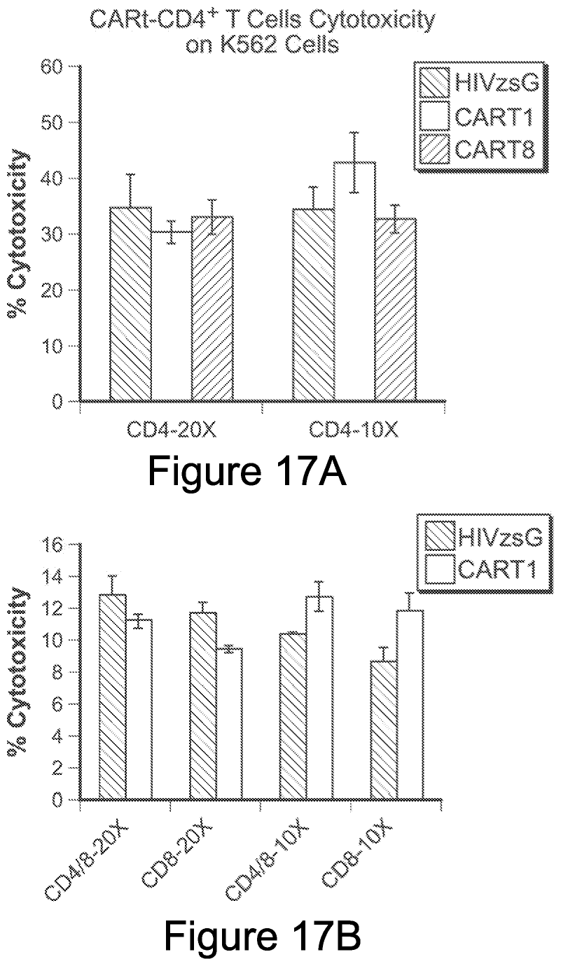

Primary T cell populations were isolated from PMBCs obtained from donors by FACS by positively selecting CD4⁺, CD8⁺, or CD4⁺/CD8⁺ cells, resulting in highly pure populations (FIG. 16, panels A and B). Each of the populations of primary T cells (CD4⁺, CD8⁺, or CD4⁺/CD8⁺ cells) were transduced with a lentiviral vector containing the chimeric receptors (e.g., CART1 and CART8) and the resulting primary T cells expressing chimeric receptors were used to perform cytotoxicity assays, as described above. Co-incubation of the population of CD4⁺ T cells expressing the chimeric receptors with K562 (1000 target K562 cells) did not result in cytotoxicity of the K562 cells (FIG. 17, panel A). In contrast, in a cytotoxicity assay using either CD8⁺ or CD4⁺/CD8⁺ cells expressing the chimeric receptors and 1000 target K562 cells, the CD8⁺ or CD4⁺/CD8⁺ cells were able to induce cell death of the K562 cells at a low cellular ratio (FIG. 17, panel B).

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one of skill in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A genetically engineered hematopoietic stem or progenitor cell, which comprises an altered splice acceptor or exonic splicing enhancer site in exon 2 of an endogenous CD33 gene, and wherein the alteration causes a reduced expression level of an epitope encoded by exon 2 of CD33 as compared with a wild-type counterpart cell.

2. The genetically engineered hematopoietic stem or progenitor cell of claim 1, wherein the alteration is a nucleotide substitution in the splice acceptor or exonic splicing enhancer site in exon 2 of CD33 or a nucleotide substitution in the nucleotide sequence of the intron 1/exon 2 junction of CD33.

3. (canceled)

4. The genetically engineered hematopoietic stem or progenitor cell of claim 1, wherein the alteration is a nucleotide substitution chosen from the group consisting of: C to T; G to A; A to G; and T to C.

5. A genetically engineered hematopoietic stem or progenitor cell, which comprises
(i) an altered splice donor site in exon 13 of an endogenous EMR2 gene, and wherein the alteration causes a reduced expression level of an epitope encoded by exon 13 of EMR2 as compared with a wild-type counterpart cell;
(ii) an altered splice acceptor or exonic splicing enhancer site in an exon of a first endogenous gene which encodes a lineage-specific antigen and an altered splice acceptor or exonic splicing enhancer site in an exon of a second endogenous gene which encodes a lineage-specific antigen, wherein the alterations cause a reduced expression level of an epitope encoded by the exon of the first endogenous gene and/or a reduced expression of an epitope encoded by the exon of the second endogenous gene, as compared with a wild-type counterpart cell; or
(iii) at least one nucleotide substitution in a gene encoding a lineage-specific antigen, wherein the nucleotide substitution is comprised within a sequence encoding a splice element, wherein the nucleotide substitution results in an alternative splicing of a transcript encoded by the gene, and wherein the alternative splicing causes a reduced expression level of an epitope encoded by the gene as compared with a wild-type counterpart cell, and wherein the epitope is targeted by an immunotherapeutic agent.

6. The genetically engineered hematopoietic stem or progenitor cell of claim 5, wherein the alteration is a nucleotide substitution in the splice donor site in exon 13 of EMR2 or a nucleotide substitution in the nucleotide sequence of the intron 12/exon 13 junction of EMR2.

7-15. (canceled)

16. The genetically engineered hematopoietic stem or progenitor cell of claim 1, which is CD34+.

17-19. (canceled)

20. The genetically engineered hematopoietic stem or progenitor cell of claim 1, which does not comprise a mutation in any predicted off-target site.

21. A cell population, comprising a plurality of the genetically engineered hematopoietic stem or progenitor cell of claim 1.

22. A method of producing a genetically engineered hematopoietic stem or progenitor cell, which comprises at least one nucleotide substitution in a gene encoding a lineage-specific antigen and optionally a second lineage-specific antigen, comprising:
(i) providing a hematopoietic stem or progenitor cell, and
(ii) introducing into the cell: (a) a guide RNA (gRNA) that comprises a targeting domain targeting a nucleotide sequence within the genome of the hematopoietic stem or progenitor cell that comprises a splice element; and (b) a catalytically impaired Cas9 endonuclease fused to a cytosine or adenosine deaminase (base editor); and optionally
(iii) further introducing into the cell (a) a second guide RNA (gRNA) that comprises a targeting domain targeting a second nucleotide sequence within the genome of the hematopoietic stem or progenitor cell that comprises a splice element; and (b) a second catalytically impaired Cas9 endonuclease) fused to cytosine or adenosine deaminase (base editor),
thereby producing a genetically engineered hematopoietic stem or progenitor cell.

23. The method of claim 22, wherein the splice element is chosen from the group consisting of a splice acceptor, splice donor, splice enhancer and splice silencer.

24. The method of claim 22, wherein the at least one nucleotide substitution in a gene causes alternative splicing: optionally wherein alternative splicing causes an exon encoding an epitope to be skipped or wherein the at least one nucleotide substitution in a gene causes alternative splicing causes an exon encoding an epitope to be extended.

25-26. (canceled)

27. The method of claim 24, wherein the nucleotide substitution is chosen from the group consisting of: C to T; G to A; A to G; and T to C.

28. The method of claim 22, wherein the gRNA comprises the sequence chosen from the group consisting of SEQ ID NOs: 1-4 and 46-47.

29. The method of claim 22, wherein the lineage-specific antigen is CD33 or EMR2.

30-38. (canceled)

39. The method of claim 22, wherein the base editor is a cytosine base editor or an adenosine base editor.

40. (canceled)

41. The method of claim 22, wherein (a) and (b) are encoded on one vector, which is introduced into the cell: or wherein (b) is in protein form and (a) and (b) are introduced into the cell as a pre-formed ribonucleoprotein complex.

42-44. (canceled)

45. The method of claim 22, wherein the hematopoietic stem or progenitor cell is CD34+ and/or the hematopoietic stem or progenitor cell is from bone marrow cells or peripheral blood mononuclear cells (PBMCs) of a subject.

46-48. (canceled)

49. A method of treating a hematopoietic disorder, comprising administering to a subject in need thereof an effective amount of the genetically engineered hematopoietic stem or progenitor cell of claim 1; optionally wherein the hematopoietic disorder is a hematopoietic malignancy.

50. (canceled)

51. The method of claim 49, further comprising administering to the subject an effective amount of an agent that targets a lineage-specific antigen, and wherein the agent comprises an antigen-binding fragment that binds the lineage-specific antigen, optionally wherein the agent that targets the lineage-specific antigen is an immune cell expressing a chimeric antigen receptor (CAR), which comprises the antigen-binding fragment that binds the lineage-specific antigen.

52-57. (canceled)

58. The method of claim 49, wherein the subject is a human patient having Hodgkin's lymphoma, non-Hodgkin's lymphoma, leukemia, or multiple myeloma; optionally wherein the subject is a human patient having leukemia, which is acute myeloid leukemia, chronic myelogenous leukemia, acute lymphoblastic leukemia, or chronic lymphoblastic leukemia.

59. (canceled)
